(19) United States
(12) Patent Application Publication
(10) Pub. No.: US 2025/0057963 A1
(43) Pub. Date: Feb. 20, 2025

(54) DISCOVERY OF PIPERLONGUMINE AS A NOVEL E3 LIGASE LIGAND

(71) Applicant: University of Florida Research Foundation, Incorporated, Gainesville, FL (US)

(72) Inventors: Wanyi Hu, Gainesville, FL (US); Xingui Liu, Gainesville, FL (US); Dongwen Lyu, Gainesville, FL (US); Jing Pei, Gainesville, FL (US); Yufeng Xiao, Gainesville, FL (US); Yaxia Yuan, Gainesville, FL (US); Guangrong Zheng, Gainesville, FL (US); Daohong Zhou, Gainesville, FL (US)

(73) Assignee: University of Florida Research Foundation, Incorporated, Gainesville, FL (US)

(21) Appl. No.: 18/720,572

(22) PCT Filed: Dec. 16, 2022
(86) PCT No.: PCT/US2022/053112

§ 371 Date: Jun. 14, 2024

Related U.S. Application Data
(60) Provisional application No. 63/291,319, filed on Dec. 17, 2021.

(51) Int. Cl. A61K 47/55; A61P 35/00

(52) U.S. Cl. CPC A61K 47/55; A61P 35/00

(57) ABSTRACT

Provided herein are compounds (e.g., compounds of Formula (I)), their mechanism of action, and methods of treating diseases and disorders (e.g., cancer) using the compounds provided herein (e.g., compounds of Formula (I)). Also disclosed herein are N methods of inhibiting and degrading kinases (e.g., CDK9, CDK10, or anaplastic lymphoma kinase).

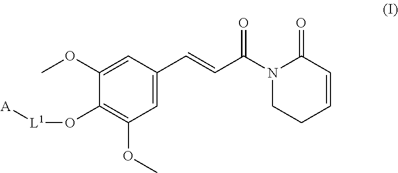

### RELATED APPLICATION

The present application claims priority to U.S. Provisional Patent Application No. 63/291,319, filed Dec. 17, 2021, which is incorporated herein by reference in its entirety.

### GOVERNMENT SUPPORT

This invention was made with government support under grants R01 AG063801, R56 AG065635, R01 CA242003, and R01 CA241191 awarded by the National Institutes of Health.

The government has certain rights in the invention.

### BACKGROUND

PROteolysis TArgeting Chimeras (PROTAC) is a new modality to delete oncoproteins in an event driving manner. Compared with traditional small molecule inhibitors which only block the catalytic function of POIs, PROTACs can further remove their scaffold function through inducing the complete degradation of POIs from the cell. These advantages are more obvious to those proteins without traditionally binding pocket which were considered as undruggable proteins previously. For example, a potent signal transducer and activator of transcription 3 (STAT3) PROTAC has been reported and shown efficacy in vivo. In addition, PROTAC induced POI degradation is driven by the ternary complex formation and this property has been demonstrated to convert a promiscuous inhibitor to a more specific degrader. A previous study demonstrated lysine availability provides another layer of selectivity.

Although progress has been made in the field of small molecule PROTAC, there are still some obstacles. Among them, very limited E3 ligases and their ligands can be used to generate PROTACs. The human genome encodes more than 600 E3 ligases and only several E3 ligases (CRBN, VHL, cIAPs, and MDM2) have been commonly utilized by PROTAC to degrade POIs. However, only a few E3 ligases have been used for PROTAC design. The few available E3 ligases for PROTAC design limits the ability to generate PROTACs for a POI that is not a neo-substrate for those E3 ubiquitin ligases because different proteins may require different E3 ligases to mediate their degradation. Recent studies have also shown that cancer cells might be resistant to PROTACs through CUL2 loss for VHL-based bromodomain and extra-terminal domain (BET) PROTAC or CRBN loss for CRBN-based BET and CDK9 PROTACs. Thus, the development of new E3 ligase ligands is an urgent task.

### SUMMARY OF THE INVENTION

It is now discovered that compounds provided herein possess heretofore unknown functional activity relevant for treating a disease or disorder (e.g., cancer). The present disclosure relates to methods of treating or preventing a disease or disorder (e.g., cancer). The present disclosure also provides methods of degrading kinases (e.g., CDK9, CDK10, or anaplastic lymphoma kinase).

In one aspect, provided herein is a compound of Formula (I),

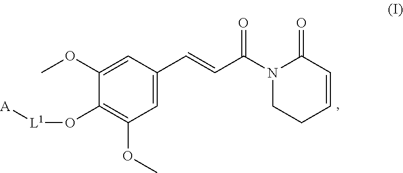

or a pharmaceutically acceptable salt, hydrate, solvate, tautomer, or prodrug thereof, wherein A and L¹ are as defined herein.

In another aspect, provided herein is a pharmaceutical composition comprising a compound provided herein (e.g., a compound of Formula (I)), or a pharmaceutically acceptable salt, hydrate, solvate, tautomer, or prodrug thereof.

In another aspect, provided herein is a method of preventing or treating a disease or disorder in a subject in need thereof, the method comprising administering an effective amount of a compound provided herein (e.g., a compound of Formula (I)), or a pharmaceutically acceptable salt, hydrate, solvate, tautomer, or prodrug thereof, or a pharmaceutical composition provided herein.

In another aspect, provided herein is a method of preventing or treating a subject suffering from or susceptible to a disease or disorder, the method comprising administering an effective amount of a compound provided herein (e.g., a compound of Formula (I)), or a pharmaceutically acceptable salt, hydrate, solvate, tautomer, or prodrug thereof, or a pharmaceutical composition provided herein.

In another aspect, provided herein is a use of a compound provided herein (e.g., a compound of Formula (I)), or a pharmaceutically acceptable salt, hydrate, solvate, tautomer, or prodrug thereof, or a pharmaceutical composition thereof for the preparation of a medicament for preventing or treating a disease or disorder in a subject in need thereof.

In another aspect, provided herein is a use of a compound provided herein (e.g., a compound of Formula (I)), or a pharmaceutically acceptable salt, hydrate, solvate, tautomer, or prodrug thereof, or a pharmaceutical composition thereof for the preparation of a medicament for preventing or treating a disease or disorder in a subject suffering from or susceptible to a disease or disorder.

In another aspect, provided herein is a compound provided herein (e.g., a compound of Formula (I)), or a pharmaceutically acceptable salt, hydrate, solvate, tautomer, or prodrug thereof, or a pharmaceutical composition thereof for use in preventing or treating a disease or disorder in a subject in need thereof.

In another aspect, provided herein is a compound provided herein (e.g., a compound of Formula (I)), or a pharmaceutically acceptable salt, hydrate, solvate, tautomer, or prodrug thereof, or a pharmaceutical composition thereof for use in preventing or treating a disease or disorder in a subject suffering from or susceptible to a disease or disorder.

In some embodiments, the disease is cancer. In certain embodiments, the cancer is a solid tumor or liquid tumor. In some embodiments, the cancer is non-small cell lung cancer. In certain embodiments, the cancer is prostate cancer.

In another aspect, provided herein is a method of inhibiting a kinase, the method comprising contacting a kinase with an effective amount of a compound provided herein (e.g., a compound of Formula (I)), or a pharmaceutically acceptable salt, hydrate, solvate, tautomer, or prodrug thereof.

In another aspect, provided herein is a method of degrading a kinase, the method comprising contacting a kinase with an effective amount of a compound provided herein (e.g., a compound of Formula (I)), or a pharmaceutically acceptable salt, hydrate, solvate, tautomer, or prodrug thereof.

In some embodiments, the kinase is a CDK (cyclin-dependent kinase). In certain embodiments, the kinase is CDK9. In some embodiments, the kinase is CDK10. In certain embodiments, the kinase is anaplastic lymphoma kinase.

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

FIGS. 1A to 1D show methods of identifying PL-binding E3 ligases. FIG. 1A shows the structures of PL and PL-Alkyne probe. FIG. 1B depicts the workflow of the competitive ABPP assay. FIG. 1C shows Western blot to detect biotin-labeled proteins in the streptavidin pull-downed samples. FIG. 1D shows the PL-binding proteins identified by mass spectrometry, wherein E3 ligases are shown underlined and GSTO1 is shown boxed. MOLT4 is a human T-cell acute lymphoblastic leukemia (T-ALL) cell line.

FIGS. 2A to 2F depicts PL-SNS-032 conjugate 955 potently induces CDK9 degradation and apoptosis. (FIG. 2A) The structure of 955; (FIG. 2B) 955 but not SNS-032 induces the degradation of CDK9 and is more potent than SNS-032 in the induction of PARP cleavage; (FIG. 2C) Time course of 955-induced CDK9 degradation and PARP cleavage; (FIG. 2D) 955 induces CDK9 degradation and PARP cleavage for up to 18 h after washout of 955; (FIG. 2E) PL alone or in combination with SNS-032 does not induce CDK9 degradation; and (FIG. 2F) Pre-treatment with SNS-032 or PL blocks the CDK9 degradation induced by 955. All the experiments were performed in MOLT4 cells. Representative immunoblots are shown and (3-actin was used as an equal loading control in all immunoblot analyses. FL PARP (full-length PARP) and CL PARP (cleaved PARP) are used as indicators of apoptosis. The quantification of the relative CDK9 protein content in the immunoblots is presented as mean±SD (n=2 biologically independent experiments) in the bar graph (bottom panel). Statistical significance was calculated with unpaired two-tailed Student's t-tests. *P<0.05; **P<0.01.

FIGS. 3A to 3H depict a mechanism of action of 955 in degrading CDK9. (FIG. 3A) Pre-treatment with the proteasome inhibitor MG132 or bortezomib blocks CDK9 degradation by 955; (FIG. 3B) Pre-treatment with the lysosome inhibitor Baf-A1 or chloroquine does not block CDK9 degradation by 955; (FIG. 3C) Pre-treatment with pan-caspase inhibitor QVD does not block CDK9 degradation by 955; (FIG. 3D) Pre-treatment with E1 inhibitor PYR-41 or TAK-243 blocks CDK9 degradation by 955; (FIG. 3E, bottom two panels) Pre-treatment with neddylation inhibitor MLN4924 blocks CDK9 degradation by 955. (FIG. 3E, top two panels) The structure of 336 illustrates the reduction of the C2-C3 and C7-C8 double bonds of PL in comparison with 955; (FIG. 3F) the structure of 336 in which the two double carbon bonds of PL (indicated by arrows) were removed as compared with compound 955. All the experiments were performed in MOLT4 cells. Representative immunoblots are shown and 3-actin was used as a loading control in all immunoblot analyses. The quantification of the relative CDK9 protein content in the immunoblots is presented as mean±SD (n=2 biologically independent experiments) in the bar graph (bottom panel). Statistical significance was calculated with unpaired two-tailed Student's t-tests. *P<0.05; **P<0.01; NS: not significant. FIGS. 3G and 3H show immunoblots demonstrating that 336 cannot degrade CDK9 in MOLT4 cells. Representative immunoblots are shown and 3-actin was used as a loading control in all immunoblot analyses.

FIGS. 4A to 4J depict identification of CDK9 degrading E3 ligase(s) recruited by 955. (FIG. 4A) Schematic illustration of the TurboID-bait assay to identify CDK9 degrading E3 ligase(s) recruited by 955; (FIG. 4B) The biotinylated proteins identified by MS in 293T cells. E3 ligases are labelled with KEAP1; (FIG. 4C) Western blot analysis to validate KEAP1 recruitment by 955. Representative immunoblots from two independent experiments are shown. (FIG. 4D) KEAP1 inhibition with CDDO-ME and CDDO-IM blocks 955-induced CDK9 degradation in MOLT4 cells; (FIG. 4E) Inhibition of KEAP1 with DMF blocks 955-induced CDK9 degradation in MOLT4 cells. (FIG. 4F) KEAP1 knockout blocks 955-induced CDK9 degradation. KEAP1 depleted (sg1 and sg2) and control (sgNC) H1299 cells were either untreated or treated with 955 for 16 h. (FIG. 4G) Ectopic expression of Halo-tagged KEAP1 in KEAP1 stable knockout cells rescues 955-induced CDK9 degradation. KEAP1-sg1 stable knockout H1299 cells were transfected with or without Halo-KEAP1 for 48 h and then the cells were reseeded and treated with 955 for 6 h. Representative immunoblots are shown and j-actin was used as a loading control in all immunoblot analyses. In FIGS. 4D to 4G, representative immunoblots are shown and j-actin was used as an equal loading control. (FIG. 4H) Gel-based ABPP assay demonstrates that 955 competes for IA-Alkyne binding to KEAP1 in a dose-dependent manner. Representative immunoblots from two independent experiments are shown. (FIG. 4I) nanoBRET assay demonstrates that 955, but not 336, can induce the formation of the intracellular ternary complexes in live 293T cells. Data are expressed as mean±SD of three biological replicates. FIG. 4J shows immunoblots validating the MS results identifying E3 ligases.

FIGS. 5A to 5H show that 955 selectively degrades CDK9 and CDK10 by proteomic studies. (FIGS. 5A to 5C) Proteome changes induced by 955 0.1 μM 1 h (FIG. 5A), 955 0.1 μM 6 h (FIG. 5B), or SNS-032 1 μM 6 h (FIG. 5C) in MOLT4 cells. Gray dots: shared differentially expressed proteins after 0.1 μM 955 or 1 μM SNS032 treatment for 6 h. (FIG. 5D) 955, but not SNS-032, degrades CDK10 in MOLT4 cells. (FIG. 5E) Pre-treatment with MG132 and MLN4924 blocks CDK10 degradation induced by 955. In FIGS. 5D and 5E, representative immunoblots are shown and 3-actin was used as an equal loading control. FIG. 5F shows percent relative viability as a function of the dose, demonstrating 955 is more potent to kill MOLT4 cells compared with SNS-032. FIG. 5G shows EC₅₀ for various compounds, demonstrating that 955 is more potent to kill multiple prostate cancer cells. MTS assays were performed to measure the anti-proliferation effect of indicated compound(s) after 72 h treatment. FIG. 5H shows an immunoblot demonstrating that 955 induces CDK9 degradation in prostate cancer LNCaP cells. The downstream proteins (c-Myc and MCL-1) of CDK9 were down-regulated after 955-induced CDK9 degradation. Representative immunoblots are shown and 3-actin was used as a loading control in all immunoblot analyses. In FIG. 5H, representative immunoblots are shown and 0-actin was used as an equal loading control. The quantification of the relative CDK9, c-Myc, or MCL-1 protein content in the immunoblots is presented as mean±SD (n=2 biologically independent experiments) in the bar graph (right panel). Statistical significance was calculated with unpaired two-tailed Student's t-tests. *P<0.05; **P<0.01.

FIGS. 6A to 6D shows the PL-Ceritinib conjugate degrades the EML4-ALK-fusion protein. FIG. 6A shows the structure of PL-ceritinib compounds 819A (819), 819B, 819C, and 819D. (FIG. 6B) 819 but not ceritinib degrades EML4-ALK in NCI-H2228 cells; (FIG. 6C) Pre-treatment with the neddylation inhibitor MLN4924 or proteasome inhibitor MG132 blocks EML4-ALK degradation induced by 819; and (FIG. 6D) Pre-treatment with the KEAP1 inhibitor DMF blocks the 819-induced EML4-ALK degradation. In panels B, C and D, representative immunoblots are shown and 3-actin was used as an equal loading control. The quantification of the relative EML4-ALK protein content in the immunoblots is presented as mean±SD (n=2 biologically independent experiments) in the bar graph (bottom panel). Statistical significance was calculated with unpaired two-tailed Student's t-tests. *P<0.05; NS: not significant

FIG. 7 shows a schematic of PL-SNS-032 conjugated compound 955 recruiting KEAP1 to potently degrade CDK9 and kill cancer cells

FIGS. 8A to 8C show GO and KEGG enrichment analysis of PL binding proteins identified by competitive ABPP and LC-MS/MS. FIG. 8A shows enriched GO BP items. FIG. 8B shows enriched GO MF items. FIG. 8C shows enriched KEGG items.

FIGS. 9A to 9C show synthesis and evaluation of a series of PL-SNS-032 conjugates. FIG. 9A shows the structure of the PL-SNS-032 conjugates. FIG. 9B shows evaluation of the PL-SNS-032 conjugates in MOLT4 cells. The DC₅₀ and Dₘₐₓ were calculated using Prism based on the ImageJ-quantified Western blots in FIG. 9C and the EC50 were determined by MTS assays. For the MTS assays, the data are presented as mean±SD from three independent experiments. FIG. 9C shows the CDK9 degradation profiling of the PL-SNS-032 conjugates evaluated in MOLT4 cells. Representative immunoblots are shown and 3-actin was used as a loading control in all immunoblot analyses. The quantification of the relative CDK9 protein content in the immunoblots is presented as mean±SD (n=2 biologically independent experiments) in the bar graph (bottom panel). Statistical significance was calculated with unpaired two-tailed Student's t-tests. *P<0.05; **P<0.01.

FIGS. 10A to 10D show 955 degrades CDK9 and downregulates its downstream proteins. FIG. 10A shows an immunoblot demonstrating 955 induces the degradation of CDK9 and downregulation of its downstream proteins in MOLT4 cells. FIG. 10B shows and immunoblot demonstrating that 955 induces the degradation of CDK9 in a dose dependent manner in MOLT4 cells. FIG. 10C shows an immunoblot demonstrating 955 induces the degradation of CDK9 in a time and dose dependent manner in 293T cells. 293T cells were treated with 0.1 μM or 1 μM 955 for indicated time. FIG. 10D shows an immunoblot demonstrating 955 induces the degradation of CDK9 in a time and dose dependent manner in K562 cells. K562 cells were treated with 0.1 μM 955 for indicated time. In FIGS. 10C and 10D, representative immunoblots are shown and (3-actin was used as a loading control in all immunoblot analyses. The quantification of the relative CDK9 protein content in the immunoblots is presented as mean±SD (n=2 biologically independent experiments) in the bar graph (bottom panel). Statistical significance was calculated with unpaired two-tailed Student's t-tests. *P<0.05; **P<0.01.

FIGS. 11A to 11F show immunoblots demonstrating 955 induces CDK9 degradation in a UPS dependent manner in 293T and K562 cells. FIGS. 11A and 11D show immunoblots demonstrating pretreatment with proteasome inhibitor MG132 or Bortezomib blocks the CDK9 degradation by 955 in 293T (FIG. 11A) and K562 (FIG. 11D) cells. FIGS. 11B and 11E show immunoblots demonstrating that pretreatment with neddylation inhibitor MLN4924 blocks the CDK9 degradation by 955 in 293T (FIG. 11B) and K562 (FIG. 11E) cells. FIGS. 11C and 11F show immunoblots demonstrating pretreatment with SNS-032 or PL blocks the CDK9 degradation by 955 in 293T (FIG. 11C) and K562 (FIG. 11F) cells. Representative immunoblots are shown in FIGS. 11A to 11F, and β-actin was used as a loading control in all immunoblot analyses. The quantification of the relative CDK9 protein content in the immunoblots is presented as mean±SD (n=2 biologically independent experiments) in the bar graph (bottom panel). Statistical significance was calculated with unpaired two-tailed Student's t-tests. *P<0.05; **P<0.01; NS: not significant.

FIGS. 12A to 12F show a comparison of the effects of SNS-032 and 955. FIG. 12A depicts a Venn diagram to show the differentially expressed genes in MOLT4 cells after 955 and SNS-032 treatments. FIG. 12B shows a heatmap to show differentially expressed genes under 955 and SNS-032 treatments. FIG. 12C shows a heatmap to show the changes of significant differentially expressed proteins identified by TMT proteomics and their corresponding changes at the transcriptional level. FIG. 12D shows validation of RNA-seq results using q-PCR to quantify MYC and MCL. FIG. 12E and FIG. 12F show that the effects of 955 and SNS-032 on the expression of the two important CDK9 downstream targets MYC and MCL1 in MOLT4 cells at the levels of transcription and translation determined by qPCR and immunoblot, respectively. The qPCR data are expressed as mean±SD of three biological replicates. Statistical significance was calculated with unpaired two-tailed Student's t-tests. *P<0.05; **P<0.01; ***P<0.001. In FIG. 12F, representative immunoblots are shown and β-actin was used as an equal loading control. The quantification of the relative CDK9, c-Myc, or MCL-1 protein content in the immunoblots is presented as mean±SD (n=2 biologically independent experiments) in the bar graph (right panel). Statistical significance was calculated with unpaired two-tailed Student's t-tests. *P<0.05; **P<0.01.

FIGS. 13A to 13B. RNA-seq analysis of the transcriptional changes in MOLT4 cells treated with 0.1 M 955 or 1 M SNS-032 for 6 h. GO enrichment analysis of down- (FIG. 13A) and up- (FIG. 13B) regulated DEGs (differentially expressed genes) identified from 955 and SNS-032 treatments. GO BP items with FDR<0.05 were extracted and shown.

FIG. 14 depicts Proteolysis-Targeting Chimera (PROTAC) degradation of protein in a Ubiquitin-Proteosome system (UPS)-dependent manner.

FIG. 15 shows the natural origin and agricultural and pharmaceutical uses of PL.

FIGS. 16A to 16D show GO and KEGG enrichment analysis of PL binding proteins. (FIG. 16A) Enriched GO BP items; (FIG. 16B) Enriched GO MF items; (FIG. 16C) Enriched KEGG items. The GO items or KEGG items with FDR<0.05 were extracted and shown. (FIG. 16D) The results show that many of the proteins involved in the UPS were highly enriched.

FIGS. 17A to 17D show KEAP1 knockdown blocks 955-induced CDK9 degradation. KEAP1 (FIG. 17A), TRIP12 (FIG. 17B), or TRAF6 (FIG. 17C) was knocked down by siRNAs in H1299 cells and then the cells were either untreated or treated with indicated concentration of 955 for 6 h. (FIG. 17D) KEAP1 single or KEAP1 and NRF2 double knockdown by siRNAs in H1299 cells and then the cells were either untreated or treated with indicated concentration of 955 for 6 h. Representative immunoblots are shown and β-actin was used as a loading control in all immunoblot analyses. The quantification of the relative CDK9 protein content in the immunoblots is presented as mean±SD (n=2 biologically independent experiments) in the bar graph (bottom panel). Statistical significance was calculated with unpaired two-tailed Student's t-tests. *P<0.05; **P<0.01; NS: not significant.

FIG. 18 is a Venn diagram to show the differentially expressed proteins in MOLT4 cells after 6 h of treatment with 955 and SNS-032.

FIGS. 19A to 19B show percentages of cell killing in LNCaP (FIG. 19A) and H1299 (FIG. 19B) cells with or without KEAP1 knockdown. LNCaP and H1299 cells were treated with control siRNA or siRNAs targeting KEAP1 for 48 h and then reseeded and treated with indicated compound (0.1 mM for LNCaP cells and 0.3 mM for H1299 cells) for 24 h. The cell viability was then determined by MTS assay. The data are presented as mean±SD from three replicate cell cultures in a representative experiment. Data are representative of two independent experiments. Statistical significance was calculated with unpaired two-tailed Student's t-tests. **P<0.01; ***P<0.001; NS: not significant.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

When a range of values is listed, it is intended to encompass each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁-2, C₂₋₆, C₂₋₅, C₂₋₄, C₂₋₃, C₃₋₆, C₃₋₅, C₃₋₄, C₄₋₆, C₄₋₅, and C₅₋₆ alkyl.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays. The term “isotopes” refers to variants of a particular chemical element such that, while all isotopes of a given element share the same number of protons in each atom of the element, those isotopes differ in the number of neutrons.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂-6 alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

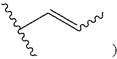

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁-s alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁-s alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃_carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁), spiro[5.5]undecanyl (C₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃_s cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅_6 cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction.

Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR—, —ON(R^{bb})₂, —N(R^{bb})₂, —N(R^{bb})₃⁺X⁻, —N(OR^{cc})R^{bb}, —SH, —SR—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(=NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(=O)(R^{aa})₂, —NR^{bb}P(=O)(OR^{cc})₂, —NR^{bb}P(=O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, =NNR^{bb}C(═O)R—, =NNR^{bb}C(═O)OR^{aa}, =NNR^{bb}S(=O)₂R^{aa}, =NR^{bb}, or =NOR^{cc};
wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R′)₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁20 alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(=NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form =O or =S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of Ra is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two Ra groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(=O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form =O or =S; and each X—is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R—, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R—, —NR^{bb}CO₂R—, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR—, —SR—, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, each nitrogen atom substituent is independently a nitrogen protecting group, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{aa}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups.

In certain embodiments, each nitrogen protecting group is independently formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, phenacylsulfonamide, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salt, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidate, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), —OH, —OR^{aa}, —N(R^{cc})₂, —C(═O)R^{aa}, —C(═O)N(R^{cc})₂, —CO₂R^{aa}, —SO₂Ra, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{cc}, C₁₋₁₀ alkyl, C₂-10 alkenyl, C₂₋₁₀ alkynyl, C₃₋₁₀ carbocyclyl, 3-14 membered heterocyclyl, C₆₋₁₄ aryl, or 5-14 membered heteroaryl, wherein each alkyl, alkenyl, alkynyl, carbocyclyl, heterocyclyl, aralkyl, aryl, and heteroaryl is independently substituted with 0, 1, 2, 3, 4, or 5 R^{dd} groups.

In certain embodiments, each oxygen atom substituent is independently an oxygen protecting group, —R^{aa}, —N(R^{bb})₂, —C(═O)SR^{aa}, —C(═O)R—, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, or —P(═O)(N(R^{bb})₂)₂.

In certain embodiments, each oxygen protecting group is independently methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, or tosylate (Ts).

In certain embodiments, each sulfur atom substituent is independently a sulfur protecting group, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R′)₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, or —P(═O)(N(R^{bb})₂)₂.

In certain embodiments, each sulfur protecting group is independently acetamidomethyl, t-Bu, 3-nitro-2-pyridine sulfenyl, 2-pyridine-sulfenyl, or triphenylmethyl.

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(=NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(=NR^{bb})N(R^{bb})₂, —NR^{bb}SO₂R—, —NR^{bb}P(=O)(OR^{cc})₂, and —NR^{bb}P(=O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X—are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, and can be further attached to a moiety to form, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(=NR^{bb})OR^{aa}), —C(═NR^{bb})N(R^{bb})₂), wherein R^{aa} and R^{bb} are as defined herein.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors. The disclosure is not intended to be limited in any manner by the above exemplary listing of substituents. Additional terms may be defined in other sections of this disclosure.

### Other Definitions

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N+(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+(C₁₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present disclosure. The compounds herein may also be represented in multiple tautomeric forms, in such instances, the invention expressly includes all tautomeric forms of the compounds described herein, even though only a single tautomeric form may be represented. While compounds may be depicted as racemic or as one or more diastereoisomers, enantiomers, or other isomers, all such racemic, diastereoisomer, enantiomer, or other isomer forms of that depicted are included in the present disclosure. All such isomeric forms of such compounds herein are expressly included in the present disclosure. All crystal forms and polymorphs of the compounds described herein are expressly included in the present invention. The term “isomers” is intended to include diastereoisomers, enantiomers, regioisomers, structural isomers, rotational isomers, tautomers, and the like. For compounds which contain one or more stereogenic centers, e.g., chiral compounds, the methods of the invention may be carried out with an enantiomerically enriched compound, a racemate, or a mixture of diastereomers.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound provided herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting a kinase. In certain embodiments, a therapeutically effective amount is an amount sufficient for degrading a kinase. In certain embodiments, a therapeutically effective amount is an amount sufficient for treating cancer. In certain embodiments, a therapeutically effective amount is an amount sufficient for inhibiting a kinase and treating cancer. In certain embodiments, a therapeutically effective amount is an amount sufficient for degrading a kinase and treating cancer.

A “prophylactically effective amount” of a compound provided herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting a kinase. In certain embodiments, a prophylactically effective amount is an amount sufficient for degrading a kinase. In certain embodiments, a prophylactically effective amount is an amount sufficient for preventing cancer. In certain embodiments, a prophylactically effective amount is an amount sufficient for inhibiting a kinase and preventing cancer. In certain embodiments, a prophylactically effective amount is an amount sufficient for degrading a kinase and preventing cancer.

As used herein the term “inhibit” or “inhibition” in the context of kinases, e.g., in the context of CDK9, CDK10, or anaplastic lymphoma kinase, refers to a reduction in the activity of the kinase. In certain embodiments, the inhibition reduces, slows, halts, or prevents the activity relative to vehicle. In some embodiments, the term refers to a reduction of the level of kinase activity, e.g., CDK9, CDK10, or anaplastic lymphoma kinase activity, to a level that is statistically significantly lower than an initial level, which may, for example, be a baseline level of activity. In some embodiments, the term refers to a reduction of the level of activity, e.g., CDK9, CDK10, or anaplastic lymphoma kinase activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of activity.

The terms “polypeptide,” “peptide” and “protein” are used interchangeably herein to refer to a polymer of amino acid residues. The terms apply to amino acid polymers in which one or more amino acid residue is an artificial 5 chemical mimetic of a corresponding naturally occurring amino acid, as well as to naturally occurring amino acid polymers and non-naturally occurring amino acid polymer.

The term “protein” refers to series of amino acid residues connected one to the other by peptide bonds between the alpha-amino and carboxy groups of adjacent 10 residues.

The term “proteolysis-targeting chimera” or “PROTAC” refers to a heterobifunctional molecule capable of inducing intracellular proteolysis. In some embodiments, a PROTAC comprises an E3-ubiquitin ligase binding molecule covalently linked to a component that binds the protein targeted for degradation.

A “kinase” is a type of enzyme that transfers phosphate groups from high energy donor molecules, such as ATP, to specific substrates, referred to as phosphorylation. Kinases are part of the larger family of phosphotransferases. One of the largest groups of kinases are protein kinases, which act on and modify the activity of specific proteins. Kinases are used extensively to transmit signals and control complex processes in cells. Various other kinases act on small molecules such as lipids, carbohydrates, amino acids, and nucleotides, either for signaling or to prime them for metabolic pathways. Kinases are often named after their substrates. More than 500 different protein kinases have been identified in humans. These exemplary human protein kinases include, but are not limited to, AAK1, ABL, ACK, ACTR2, ACTR2B, AKT1, AKT2, AKT3, ALK, ALK1, ALK2, ALK4, ALK7, AMPKa1, AMPKa2, ANKRD3, ANPa, ANPb, ARAF, ARAFps, ARG, AurA, AurAps1, AurAps2, AurB, AurBps1, AurC, AXL, BARK1, BARK2, BIKE, BLK, BMPR1A, BMPR1Aps1, BMPR1Aps2, BMPR1B, BMPR2, BMX, BRAF, BRAFps, BRK, BRSK1, BRSK2, BTK, BUB1, BUBR1, CaMKla, CaMK1b, CaMK1d, CaMK1g, CaMK2a, CaMK2b, CaMK2d, CaMK2g, CaMK4, CaMKK1, CaMKK2, caMLCK, CASK, CCK4, CCRK, CDC2, CDC7, CDK10, CDK11, CDK2, CDK3, CDK4, CDK4ps, CDK5, CDK5ps, CDK6, CDK7, CDK7ps, CDK8, CDK8ps, CDK9, CDKL1, CDKL2, CDKL3, CDKL4, CDKL5, CGDps, CHED, CHK1, CHK2, CHK2ps1, CHK2ps2, CK1a, CK1a2, CK1aps1, CK1aps2, CK1aps3, CK1d, CK1e, CK1g1, CK1g2, CK1g2ps, CK1g3, CK2a1, CK2a1-rs, CK2a2, CLIK1, CLIK1L, CLK1, CLK2, CLK2ps, CLK3, CLK3ps, CLK4, COT, CRIK, CRK7, CSK, CTK, CYGD, CYGF, DAPK1, DAPK2, DAPK3, DCAMKL1, DCAMKL2, DCAMKL3, DDR1, DDR2, DLK, DMPK1, DMPK2, DRAK1, DRAK2, DYRKIA, DYRKIB, DYRK2, DYRK3, DYRK4, EGFR, EphAl, EphA10, EphA2, EphA3, EphA4, EphA5, EphA6, EphA7, EphA8, EphB1, EphB2, EphB3, EphB4, EphB6, Erkl, Erk2, Erk3, Erk3ps1, Erk3ps2, Erk3ps3, Erk3ps4, Erk4, Erk5, Erk7, FAK, FER, FERps, FES, FGFR1, FGFR2, FGFR3, FGFR4, FGR, FLT1, FLT1ps, FLT3, FLT4, FMS, FRK, Fused, FYN, GAK, GCK, GCN2, GCN22, GPRK4, GPRK5, GPRK6, GPRK6ps, GPRK7, GSK3A, GSK3B, Haspin, HCK, HER2/ErbB2, HER3/ErbB3, HER4/ErbB4, HH498, HIPK1, HIPK2, HIPK3, HIPK4, HPK1, HRI, HRIps, HSER, HUNK, ICK, IGF1R, IKKa, IKKb, IKKe, ILK, INSR, IRAK1, IRAK2, IRAK3, IRAK4, IRE1, IRE2, IRR, ITK, JAK1, JAK2, JAK3, JNK1, JNK2, JNK3, KDR, KHS1, KHS2, KIS, KIT, KSGCps, KSR1, KSR2, LATS1, LATS2, LCK, LIMK1, LIMK2, LIMK2ps, LKB1, LMR1, LMR2, LMR3, LOK, LRRK1, LRRK2, LTK, LYN, LZK, MAK, MAP2K1, MAP2K1ps, MAP2K2, MAP2K2ps, MAP2K3, MAP2K4, MAP2K5, MAP2K6, MAP2K7, MAP3K1, MAP3K2, MAP3K3, MAP3K4, MAP3K5, MAP3K6, MAP3K7, MAP3K8, MAPKAPK2, MAPKAPK3, MAPKAPK5, MAPKAPKps1, MARK1, MARK2, MARK3, MARK4, MARKps01, MARKps02, MARKps03, MARKps04, MARKps05, MARKps07, MARKps08, MARKps09, MARKps10, MARKps11, MARKps12, MARKps13, MARKps15, MARKps16, MARKps17, MARKps18, MARKps19, MARKps20, MARKps21, MARKps22, MARKps23, MARKps24, MARKps25, MARKps26, MARKps27, MARKps28, MARKps29, MARKps30, MAST1, MAST2, MAST3, MAST4, MASTL, MELK, MER, MET, MISR2, MLK1, MLK2, MLK3, MLK4, MLKL, MNK1, MNK1ps, MNK2, MOK, MOS, MPSK1, MPSK1ps, MRCKa, MRCKb, MRCKps, MSK1, MSK12, MSK2, MSK22, MSSK1, MST1, MST2, MST3, MST3ps, MST4, MUSK, MYO3A, MYO3B, MYT1, NDR1, NDR2, NEK1, NEK10, NEK11, NEK2, NEK2ps1, NEK2ps2, NEK2ps3, NEK3, NEK4, NEK4ps, NEK5, NEK6, NEK7, NEK8, NEK9, NIK, NIM1, NLK, NRBP1, NRBP2, NuaK1, NuaK2, Obsen, Obscn2, OSR1, p38a, p38b, p38d, p38g, p70S6K, p70S6Kb, p70S6Kps1, p70S6Kps2, PAK1, PAK2, PAK2ps, PAK3, PAK4, PAK5, PAK6, PASK, PBK, PCTAIRE1, PCTAIRE2, PCTAIRE3, PDGFRa, PDGFRb, PDK1, PEK, PFTAIRE1, PFTAIRE2, PHKg1, PHKg1ps1, PHKg1ps2, PHKg1ps3, PHKg2, PIK3R4, PIM1, PIM2, PIM3, PINK1, PITSLRE, PKACa, PKACb, PKACg, PKCa, PKCb, PKCd, PKCe, PKCg, PKCh, PKCi, PKCips, PKCt, PKCz, PKD1, PKD2, PKD3, PKG1, PKG2, PKN1, PKN2, PKN3, PKR, PLK1, PLK1ps1, PLK1ps2, PLK2, PLK3, PLK4, PRKX, PRKXps, PRKY, PRP4, PRP4ps, PRPK, PSKH1, PSKHlps, PSKH2, PYK2, QIK, QSK, RAF1, RAF1ps, RET, RHOK, RIPK1, RIPK2, RIPK3, RNAseL, ROCK1, ROCK2, RON, ROR1, ROR2, ROS, RSK1, RSK12, RSK2, RSK22, RSK3, RSK32, RSK4, RSK42, RSKL1, RSKL2, RYK, RYKps, SAKps, SBK, SCYL1, SCYL2, SCYL2ps, SCYL3, SGK, SgK050ps, SgK069, SgK071, SgK085, SgK110, SgK196, SGK2, SgK223, SgK269, SgK288, SGK3, SgK307, SgK384ps, SgK396, SgK424, SgK493, SgK494, SgK495, SgK496, SIK (e.g., SIK1, SIK2), skMLCK, SLK, Slob, smMLCK, SNRK, SPEG, SPEG2, SRC, SRM, SRPK1, SRPK2, SRPK2ps, SSTK, STK33, STK33ps, STLK3, STLK5, STLK6, STLK6ps1, STLK6-rs, SuRTK106, SYK, TAK1, TAO1, TAO2, TAO3, TBCK, TBK1, TEC, TESK1, TESK2, TGFbR1, TGFbR2, TIEl, TIE2, TLK1, TLK1ps, TLK2, TLK2ps1, TLK2ps2, TNK1, Trad, Trbl, Trb2, Trb3, Trio, TRKA, TRKB, TRKC, TSSK1, TSSK2, TSSK3, TSSK4, TSSKpsl, TSSKps2, TTBK1, TTBK2, TTK, TTN, TXK, TYK2, TYK22, TYRO3, TYRO3ps, ULK1, ULK2, ULK3, ULK4, VACAMKL, VRK1, VRK2, VRK3, VRK3ps, Wee1, Wee1B, Wee1Bps, Wee1ps1, Wee1ps2, Wnk1, Wnk2, Wnk3, Wnk4, YANK1, YANK2, YANK3, YES, YESps, YSK1, ZAK, ZAP70, ZC1/HGK, ZC2/TNIK, ZC3/MINK, and ZC4/NRK.

A “proliferative disease” refers to a disease that occurs due to abnormal growth or extension by the multiplication of cells (Walker, Cambridge Dictionary ofBiology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstram's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

Provided herein are compounds (e.g., compounds of Formula (I)), and pharmaceutically acceptable salts, hydrates, solvates, tautomers, and prodrugs thereof, and pharmaceutical compositions and kits thereof. The compounds provided herein are PROTACs and can therefore be used to inhibit or degrade various kinases. Also provided herein are methods of treating and/or preventing a disease or disorder (e.g., cancer) in a subject comprising administering an effective amount of a compound or composition provided herein to the subject. Other uses of the compounds and pharmaceutical compositions provided herein include methods of inhibiting and/or degrading a kinase (e.g., in a subject or cell).

### Compounds

Provided herein is a compound of Formula (I), or a pharmaceutically acceptable salt, hydrate, solvate, tautomer, or prodrug thereof:

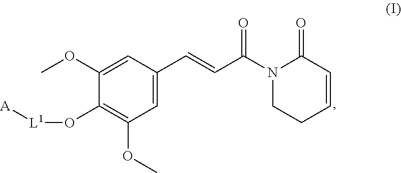

wherein:
A is a kinase inhibitor; and
L¹ is optionally substituted C₁-C₂₀ alkylene, optionally substituted C₁-C₂₀ heteroalkylene, optionally substituted C₁-C₂₀ alkenylene, optionally substituted C₁-C₂₀ heteroalkenylene, optionally substituted C₁-C₂₀ alkynylene, optionally substituted C₁-C₂₀ heteroalkynylene, optionally substituted C₃-C₁₄ carbocyclylene, or optionally substituted 3- to 14-membered heterocyclylene.

As defined herein, A is a kinase inhibitor. In certain embodiments, A is an inhibitor of a kinase provided herein. In some embodiments, A is a CDK inhibitor. In certain embodiments, A is a CDK9, CDK10, or anaplastic lymphoma kinase inhibitor. In some embodiments, A is a CDK9 or CDK10 inhibitor. In certain embodiments, A is a CDK9 inhibitor. In certain embodiments, A is a CDK inhibitor that is selective for CDK9 over some or all other CDKs (e.g., between 2- and 3-, between 3- and 5-, between 5- and 7-, between 7- and 10-, between 10- and 30-, or between 30- and 100-fold, inclusive, more potent in an in vitro assay against CDK9 than some or all other CDKs). In some embodiments, A is AT-7519, atuveciclib, AZD4573, BAY-1251152, CDKI-73, CDKI-73, dinaciclib, flavopiridol, i-CDK9, JSH-150, LDC000067, LY-2857785, NVP-2, RGB-286638, seliciclib, TG02, or zotiraciclib. In some embodiments, A is a CDK10 inhibitor. In certain embodiments, A is a CDK inhibitor that is selective for CDK10 over some or all other CDKs (e.g., between 2- and 3-, between 3- and 5-, between 5- and 7-, between 7- and 10-, between 10- and 30-, or between 30- and 100-fold, inclusive, more potent in an in vitro assay against CDK10 than some or all other CDKs). In certain embodiments, A is an anaplastic lymphoma kinase inhibitor. In some embodiments, A is

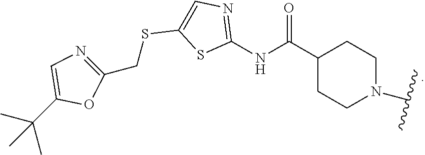 [SMILES: *N1CCC(C(=O)Nc2ncc(SCc3ncc(C(C)(C)C)o3)s2)CC1]

In certain embodiments, A is

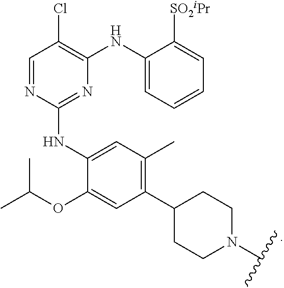

In certain embodiments, A is alectinib, AP-26113, ASP-3026, brigatinib, CEP-37440, crizotinib, ensartinib, entrectinib, lorlatinib, NMS-E628, PF-06463922, TSR-011, X-376, or X-396.

In some embodiments, L¹ comprises optionally substituted C₁-C₂₀ alkylene, optionally substituted C₁-C₂₀ heteroalkylene, optionally substituted C₁-C₂₀ alkenylene, optionally substituted C₁-C₂₀ heteroalkenylene, optionally substituted C₁-C₂₀ alkynylene, optionally substituted C₁-C₂₀ heteroalkynylene, optionally substituted C₃-C₁₄ carbocyclylene, or optionally substituted 3- to 14-membered heterocyclylene. In some embodiments, L¹ is optionally substituted C₁-C₂₀ alkylene, optionally substituted C₁-C₂₀ heteroalkylene, optionally substituted C₁-C₂₀ alkenylene, optionally substituted C₁-C₂₀ heteroalkenylene, optionally substituted C₁-C₂₀ alkynylene, optionally substituted C₁-C₂₀ heteroalkynylene, optionally substituted C₃-C₁₄ carbocyclylene, or optionally substituted 3- to 14-membered heterocyclylene.

In some embodiments, L¹ comprises optionally substituted C₁-C₁₅ alkylene, optionally substituted C₁-C₁₅ heteroalkylene, optionally substituted C₁-C₁₅ alkenylene, optionally substituted C₁-C₁₅ heteroalkenylene, optionally substituted C₁-C₁₅ alkynylene, optionally substituted C₁-C₁₅ heteroalkynylene, optionally substituted C₃-C₇ carbocyclylene, or optionally substituted 3- to 7-membered heterocyclylene. In some embodiments, L¹ is optionally substituted C₁-C₁₅ alkylene, optionally substituted C₁-C₁₅ heteroalkylene, optionally substituted C₁-C₁₅ alkenylene, optionally substituted C₁-C₁₅ heteroalkenylene, optionally substituted C₁-C₁₅ alkynylene, optionally substituted C₁-C₁₅ heteroalkynylene, optionally substituted C₃-C₇ carbocyclylene, or optionally substituted 3- to 7-membered heterocyclylene.

In some embodiments, L¹ comprises optionally substituted C₁-C₂₀ alkylene, optionally substituted C₁-C₂₀ heteroalkylene, optionally substituted C₃-C₁₄ carbocyclylene, or optionally substituted 3- to 14-membered heterocyclylene. In some embodiments, L¹ is optionally substituted C₁-C₂₀ alkylene, optionally substituted C₁-C₂₀ heteroalkylene, optionally substituted C₃-C₁₄ carbocyclylene, or optionally substituted 3- to 14-membered heterocyclylene. In some embodiments, L¹ is optionally substituted C₁-C₂₀ alkylene, optionally substituted C₁-C₂₀ heteroalkylene, or optionally substituted 3- to 14-membered heterocyclylene. In some embodiments, L¹ is optionally substituted C₁-C₂₀ alkylene or optionally substituted C₁-C₂₀ heteroalkylene. In some embodiments, L¹ is optionally substituted C₁-C₂₀ alkylene or optionally substituted C₁-C₁₄ carbocyclylene. In some embodiments, L¹ is optionally substituted C₁-C₂₀ heteroalkylene or optionally substituted 3- to 14-membered heterocyclylene. In some embodiments, L¹ is optionally substituted C₃-C₁₄ carbocyclylene or optionally substituted 3- to 14-membered heterocyclylene.

In some embodiments, L¹ comprises optionally substituted C₁-C₁₅ alkylene, optionally substituted C₁-C₁₅ heteroalkylene, optionally substituted C₃-C₇ carbocyclylene, or optionally substituted 3- to 7-membered heterocyclylene. In some embodiments, L¹ is optionally substituted C₁-C₁₅ alkylene, optionally substituted C₁-C₁₅ heteroalkylene, optionally substituted C₃-C₇ carbocyclylene, or optionally substituted 3- to 7-membered heterocyclylene. In some embodiments, L¹ is optionally substituted C₁-C₁₅ alkylene, optionally substituted C₁-C₁₅ heteroalkylene, or optionally substituted 3- to 7-membered heterocyclylene. In some embodiments, L¹ is optionally substituted C₁-C₁₅ alkylene or optionally substituted C₁-C₁₅ heteroalkylene. In some embodiments, L¹ is optionally substituted C₁-C₁₅ alkylene or optionally substituted C₃-C₇ carbocyclylene. In some embodiments, L¹ is optionally substituted C₁-C₁₅ heteroalkylene or optionally substituted 3- to 7-membered heterocyclylene. In some embodiments, L¹ is optionally substituted C₃-C₇ carbocyclylene or optionally substituted 3- to 7-membered heterocyclylene.

In some embodiments, L¹ is substituted with a carbonyl. In some embodiments, L¹ comprises C₁-C₂₀ alkylene substituted with a carbonyl or C₁-C₂₀ heteroalkylene substituted with a carbonyl.

In some embodiments, L¹ comprises optionally substituted C₁-C₂₀ alkylene. In some embodiments, L¹ is optionally substituted C₁-C₂₀ alkylene. In some embodiments, L¹ comprises substituted C₁-C₂₀ alkylene. In some embodiments, L¹ is substituted C₁-C₂₀ alkylene. In some embodiments, L¹ comprises C₁-C₂₀ alkylene substituted with a carbonyl. In some embodiments, L¹ is C₁-C₂₀ alkylene substituted with a carbonyl. In some embodiments, L¹ comprises unsubstituted C₁-C₂₀ alkylene. In some embodiments, L¹ is unsubstituted C₁-C₂₀ alkylene. In some embodiments, L¹ comprises optionally substituted C₁-C₁₅ alkylene. In some embodiments, L¹ is optionally substituted C₁-C₁₅ alkylene. In some embodiments, L¹ comprises substituted C₁-C₁₅ alkylene. In some embodiments, L¹ is substituted C₁-C₁₅ alkylene. In some embodiments, L¹ comprises C₁-C₁₅ alkylene substituted with a carbonyl. In some embodiments, L¹ is C₁-C₁₅ alkylene substituted with a carbonyl. In some embodiments, L¹ comprises unsubstituted C₁-C₁₅ alkylene. In some embodiments, L¹ is unsubstituted C₁-C₁₅ alkylene.

In some embodiments, L¹ comprises optionally substituted C₁-C₂₀ heteroalkylene. In some embodiments, L¹ is optionally substituted C₁-C₂₀ heteroalkylene. In some embodiments, L¹ comprises substituted C₁-C₂₀ heteroalkylene. In some embodiments, L¹ is substituted C₁-C₂₀ heteroalkylene. In some embodiments, L¹ comprises C₁-C₂₀ heteroalkylene substituted with a carbonyl. In some embodiments, L¹ is C₁-C₂₀ heteroalkylene substituted with a carbonyl. In some embodiments, L¹ comprises unsubstituted C₁-C₂₀ heteroalkylene. In some embodiments, L¹ is unsubstituted C₁-C₂₀ heteroalkylene. In some embodiments, L¹ comprises optionally substituted C₁-C₁₅ heteroalkylene. In some embodiments, L¹ is optionally substituted C₁-C₁₅ heteroalkylene. In some embodiments, L¹ comprises substituted C₁-C₁₅ heteroalkylene. In some embodiments, L¹ is substituted C₁-C₁₅ heteroalkylene. In some embodiments, L¹ comprises C₁-C₁₅ heteroalkylene substituted with a carbonyl. In some embodiments, L¹ is C₁-C₁₅ heteroalkylene substituted with a carbonyl. In some embodiments, L¹ comprises unsubstituted C₁-C₁₅ heteroalkylene. In some embodiments, L¹ is unsubstituted C₁-C₁₅ heteroalkylene. In some embodiments, L¹ comprises at least one nitrogen atom. In some embodiments, L¹ comprises one nitrogen atom. In some embodiments, L¹ comprises at least one oxygen atom.

In some embodiments, L¹ comprises optionally substituted C₁-C₂₀ alkenylene. In some embodiments, L¹ is optionally substituted C₁-C₂₀ alkenylene. In some embodiments, L¹ comprises substituted C₁-C₂₀ alkenylene. In some embodiments, L¹ is substituted C₁-C₂₀ alkenylene. In some embodiments, L¹ comprises C₁-C₂₀ alkenylene substituted with a carbonyl. In some embodiments, L¹ is C₁-C₂₀ alkenylene substituted with a carbonyl. In some embodiments, L¹ comprises unsubstituted C₁-C₂₀ alkenylene. In some embodiments, L¹ is unsubstituted C₁-C₂₀ alkenylene. In some embodiments, L¹ comprises optionally substituted C₁-C₁₅ alkenylene. In some embodiments, L¹ is optionally substituted C₁-C₁₅ alkenylene. In some embodiments, L¹ comprises substituted C₁-C₁₅ alkenylene. In some embodiments, L¹ is substituted C₁-C₁₅ alkenylene. In some embodiments, L¹ comprises C₁-C₁₅ alkenylene substituted with a carbonyl. In some embodiments, L¹ is C₁-C₁₅ alkenylene substituted with a carbonyl. In some embodiments, L¹ comprises unsubstituted C₁-C₁₅ alkenylene. In some embodiments, L¹ is unsubstituted C₁-C₁₅ alkenylene.

In some embodiments, L¹ comprises optionally substituted C₁-C₂₀ heteroalkenylene. In some embodiments, L¹ is optionally substituted C₁-C₂₀ heteroalkenylene. In some embodiments, L¹ comprises substituted C₁-C₂₀ heteroalkenylene. In some embodiments, L¹ is substituted C₁-C₂₀ heteroalkenylene. In some embodiments, L¹ comprises C₁-C₂₀ heteroalkenylene substituted with a carbonyl. In some embodiments, L¹ is C₁-C₂₀ heteroalkenylene substituted with a carbonyl. In some embodiments, L¹ comprises unsubstituted C₁-C₂₀ heteroalkenylene. In some embodiments, L¹ is unsubstituted C₁-C₂₀ heteroalkenylene. In some embodiments, L¹ comprises optionally substituted C₁-C₁₅ heteroalkenylene. In some embodiments, L¹ is optionally substituted C₁-C₁₅ heteroalkenylene. In some embodiments, L¹ comprises substituted C₁-C₁₅ heteroalkenylene. In some embodiments, L¹ is substituted C₁-C₁₅ heteroalkenylene. In some embodiments, L¹ comprises C₁-C₁₅ heteroalkenylene substituted with a carbonyl. In some embodiments, L¹ is C₁-C₁₅ heteroalkenylene substituted with a carbonyl. In some embodiments, L¹ comprises unsubstituted C₁-C₁₅ heteroalkenylene. In some embodiments, L¹ is unsubstituted C₁-C₁₅ heteroalkenylene. In some embodiments, L¹ comprises at least one nitrogen atom. In some embodiments, L¹ comprises one nitrogen atom. In some embodiments, L¹ comprises at least one oxygen atom.

In some embodiments, L¹ comprises optionally substituted C₁-C₂₀ alkynylene. In some embodiments, L¹ is optionally substituted C₁-C₂₀ alkynylene. In some embodiments, L¹ comprises substituted C₁-C₂₀ alkynylene. In some embodiments, L¹ is substituted C₁-C₂₀ alkynylene. In some embodiments, L¹ comprises C₁-C₂₀ alkynylene substituted with a carbonyl. In some embodiments, L¹ is C₁-C₂₀ alkynylene substituted with a carbonyl. In some embodiments, L¹ comprises unsubstituted C₁-C₂₀ alkynylene. In some embodiments, L¹ is unsubstituted C₁-C₂₀ alkynylene. In some embodiments, L¹ comprises optionally substituted C₁-C₁₅ alkynylene. In some embodiments, L¹ is optionally substituted C₁-C₁₅ alkynylene. In some embodiments, L¹ comprises substituted C₁-C₁₅ alkynylene. In some embodiments, L¹ is substituted C₁-C₁₅ alkynylene. In some embodiments, L¹ comprises C₁-C₁₅ alkynylene substituted with a carbonyl. In some embodiments, L¹ is C₁-C₁₅ alkynylene substituted with a carbonyl. In some embodiments, L¹ comprises unsubstituted C₁-C₁₅ alkynylene. In some embodiments, L¹ is unsubstituted C₁-C₁₅ alkynylene.

In some embodiments, L¹ comprises optionally substituted C₁-C₂₀ heteroalkynylene. In some embodiments, L¹ is optionally substituted C₁-C₂₀ heteroalkynylene. In some embodiments, L¹ comprises substituted C₁-C₂₀ heteroalkynylene. In some embodiments, L¹ is substituted C₁-C₂₀ heteroalkynylene. In some embodiments, L¹ comprises C₁-C₂₀ heteroalkynylene substituted with a carbonyl. In some embodiments, L¹ is C₁-C₂₀ heteroalkynylene substituted with a carbonyl. In some embodiments, L¹ comprises unsubstituted C₁-C₂₀ heteroalkynylene. In some embodiments, L¹ is unsubstituted C₁-C₂₀ heteroalkynylene. In some embodiments, L¹ comprises optionally substituted C₁-C₁₅ heteroalkynylene. In some embodiments, L¹ is optionally substituted C₁-C₁₅ heteroalkynylene. In some embodiments, L¹ comprises substituted C₁-C₁₅ heteroalkynylene. In some embodiments, L¹ is substituted C₁-C₁₅ heteroalkynylene. In some embodiments, L¹ comprises C₁-C₁₅ heteroalkynylene substituted with a carbonyl. In some embodiments, L¹ is C₁-C₁₅ heteroalkynylene substituted with a carbonyl. In some embodiments, L¹ comprises unsubstituted C₁-C₁₅ heteroalkynylene. In some embodiments, L¹ is unsubstituted C₁-C₁₅ heteroalkynylene. In some embodiments, L¹ comprises at least one nitrogen atom. In some embodiments, L¹ comprises one nitrogen atom. In some embodiments, L¹ comprises at least one oxygen atom.

In some embodiments, L¹ comprises optionally substituted C₃-C₁₄ carbocyclylene. In some embodiments, L¹ is optionally substituted C₃-C₁₄ carbocyclylene. In some embodiments, L¹ comprises optionally substituted C₃-C₇ carbocyclylene. In some embodiments, L¹ is optionally substituted C₃-C₇ carbocyclylene. In some embodiments, L¹ comprises substituted C₃-C₁₄ carbocyclylene. In some embodiments, L¹ is substituted C₃-C₁₄ carbocyclylene. In some embodiments, L¹ comprises substituted C₃-C₇ carbocyclylene. In some embodiments, L¹ is substituted C₃-C₇ carbocyclylene. In some embodiments, L¹ comprises unsubstituted C₃-C₁₄ carbocyclylene. In some embodiments, L¹ is unsubstituted C₃-C₁₄ carbocyclylene. In some embodiments, L¹ comprises unsubstituted C₃-C₇ carbocyclylene. In some embodiments, L¹ is unsubstituted C₃-C₇ carbocyclylene.

In some embodiments, L¹ comprises optionally substituted 3- to 14-membered heterocyclylene. In some embodiments, L¹ is optionally substituted 3- to 14-membered heterocyclylene. In some embodiments, L¹ comprises optionally substituted 3- to 7-membered heterocyclylene. In some embodiments, L¹ is optionally substituted 3- to 7-membered heterocyclylene. In some embodiments, L¹ comprises substituted 3- to 14-membered heterocyclylene. In some embodiments, L¹ is substituted 3- to 14-membered heterocyclylene. In some embodiments, L¹ comprises substituted 3- to 7-membered heterocyclylene. In some embodiments, L¹ is substituted 3- to 7-membered heterocyclylene. In some embodiments, L¹ comprises unsubstituted 3- to 14-membered heterocyclylene. In some embodiments, L¹ is unsubstituted 3- to 14-membered heterocyclylene. In some embodiments, L¹ comprises unsubstituted 3- to 7-membered heterocyclylene. In some embodiments, L¹ is unsubstituted 3- to 7-membered heterocyclylene. In some embodiments, the heterocyclylene comprises at least one nitrogen atom. In some embodiments, the heterocyclylene comprises one nitrogen atom. In some embodiments, the heterocyclylene comprises two nitrogen atoms. In some embodiments, the heterocyclylene comprises aziridine, azetidine, pyrrolidine, piperidine, or piperazine. In some embodiments, the heterocyclylene comprises pyrrolidine, piperidine, or piperazine. In some embodiments, the heterocyclylene comprises piperazine.

In some embodiments, L¹ is -(optionally substituted C₁-C₆ (e.g., C₁-C₃) alkylene or optionally substituted C₁-C₆ heteroalkylene)₀₋₁-(optionally substituted 3- to 7-membered heterocyclylene)-(optionally substituted C₁-C₆ (e.g., C₁-C₃) alkylene or optionally substituted C₁-C₆ heteroalkylene)₀₋₁-. In some embodiments, L¹ is -(optionally substituted C₁-C₆ (e.g., C₁-C₃) alkylene or optionally substituted C₁-C₆ heteroalkylene)₀₋₁-(optionally substituted monocyclic 6-membered heterocyclylene)-(optionally substituted C₁-C₆ (e.g., C₁-C₃) alkylene or optionally substituted C₁-C₆ heteroalkylene)₀₋₁-. In some embodiments, L¹ is -(optionally substituted C₁-C₆ (e.g., C₁-C₃) alkylene or optionally substituted C₁-C₆ heteroalkylene)₀₋₁-(optionally substituted monocyclic 6-membered para heterocyclylene)-(optionally substituted C₁-C₆ (e.g., C₁-C₃) alkylene or optionally substituted C₁-C₆ heteroalkylene)₀₋₁-.

In some embodiments, L¹ is -(optionally substituted C₁-C₆ (e.g., C₁-C₃) alkylene or optionally substituted C₁-C₆ heteroalkylene)-(optionally substituted 3- to 7-membered heterocyclylene)-(optionally substituted C₁-C₆ (e.g., C₁-C₃) alkylene or optionally substituted C₁-C₆ heteroalkylene)-. In some embodiments, L¹ is -(optionally substituted C₁-C₆ (e.g., C₁-C₃) alkylene or optionally substituted C₁-C₆ heteroalkylene)-(optionally substituted monocyclic 6-membered heterocyclylene)-(optionally substituted C₁-C₆ (e.g., C₁-C₃) alkylene or optionally substituted C₁-C₆ heteroalkylene)-. In some embodiments, L¹ is -(optionally substituted C₁-C₆ (e.g., C₁-C₃) alkylene or optionally substituted C₁-C₆ heteroalkylene)-(optionally substituted monocyclic 6-membered para heterocyclylene)-(optionally substituted C₁-C₆ (e.g., C₁-C₃) alkylene or optionally substituted C₁-C₆ heteroalkylene)-.

In some embodiments, L¹ is -(optionally substituted C₂ alkylene)₀₋₁-(optionally substituted 3- to 7-membered heterocyclylene)-(optionally substituted C₂ alkylene)₀₋₁-. In some embodiments, L¹ is -(optionally substituted C₂ alkylene)₀₋₁-(optionally substituted monocyclic 6-membered heterocyclylene)-(optionally substituted C₂ alkylene)₀₋₁-. In some embodiments, L¹ is -(optionally substituted C₂ alkylene)₀₋₁-(optionally substituted monocyclic 6-membered para heterocyclylene)-(optionally substituted C₂ alkylene)₀₋₁-.

In some embodiments, L¹ is -(optionally substituted C₂ alkylene)-(optionally substituted 3- to 7-membered heterocyclylene)-(optionally substituted C₂ alkylene)-. In some embodiments, L¹ is -(optionally substituted C₂ alkylene)-(optionally substituted monocyclic 6-membered heterocyclylene)-(optionally substituted C₂ alkylene)-. In some embodiments, L¹ is -(optionally substituted C₂ alkylene)-(optionally substituted monocyclic 6-membered para heterocyclylene)-(optionally substituted C₂ alkylene)-.

In some embodiments, L¹ is

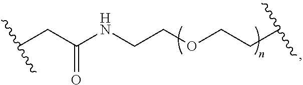

wherein n is 0, 1, 2, 3, or 4. In some embodiments, n is 0, 1, 2, or 3. In some embodiments, n is 1, 2, 3, or 4. In some embodiments, n is 0, 1, or 2. In some embodiments, n is 1, 2, or 3. In some embodiments, n is 2, 3, or 4. In some embodiments, n is 0 or 1. In some embodiments, n is 1 or 2. In some embodiments, n is 2 or 3. In some embodiments, n is 3 or 4. In some embodiments, n is 0. In some embodiments, n is 1. In some embodiments, n is 2. In some embodiments, n is 3. In some embodiments, n is 4.

In some embodiments, L¹ is

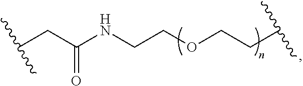

wherein n is 0, 1, 2, 3, or 4. In some embodiments, n is 0, 1, 2, or 3. In some embodiments, n is 1, 2, 3, or 4. In some embodiments, n is 0, 1, or 2. In some embodiments, n is 1, 2, or 3. In some embodiments, n is 2, 3, or 4. In some embodiments, n is 0 or 1. In some embodiments, n is 1 or 2. In some embodiments, n is 2 or 3. In some embodiments, n is 3 or 4. In some embodiments, n is 0. In some embodiments, n is 1. In some embodiments, n is 2. In some embodiments, n is 3. In some embodiments, n is 4.

In some embodiments, L¹ is

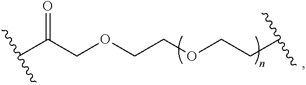

wherein n is 0, 1, 2, 3, or 4. In some embodiments, n is 0, 1, 2, or 3. In some embodiments, n is 1, 2, 3, or 4. In some embodiments, n is 0, 1, or 2. In some embodiments, n is 1, 2, or 3. In some embodiments, n is 2, 3, or 4. In some embodiments, n is 0 or 1. In some embodiments, n is 1 or 2. In some embodiments, n is 2 or 3. In some embodiments, n is 3 or 4. In some embodiments, n is 0. In some embodiments, n is 1. In some embodiments, n is 2. In some embodiments, n is 3. In some embodiments, n is 4.

In some embodiments, L¹

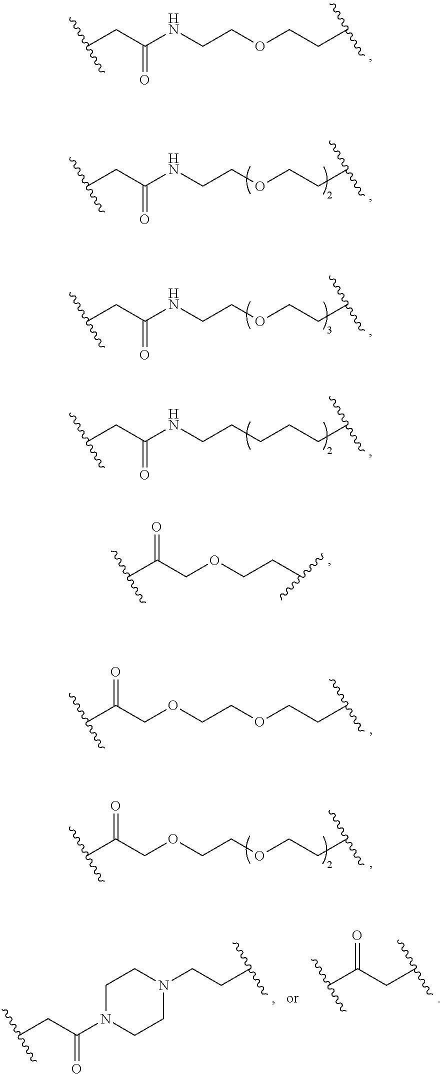

In some embodiments. L¹ is In some embodiments. L¹ is

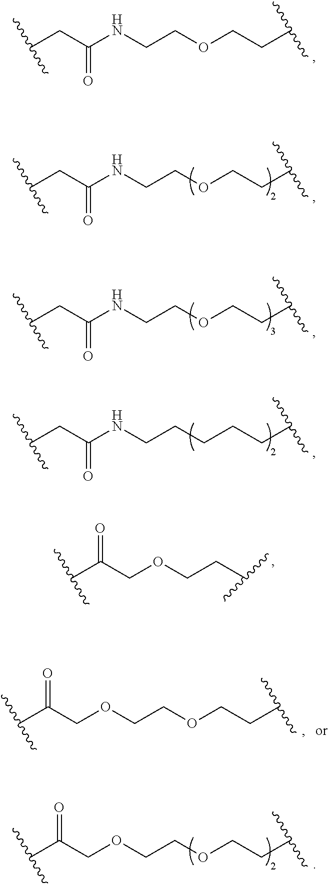

In some embodiments, L¹ is

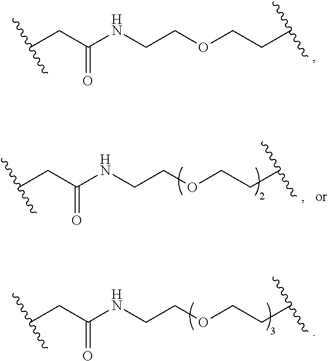

In some embodiments, L¹ is

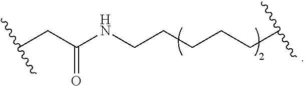

In some embodiments, L¹ is

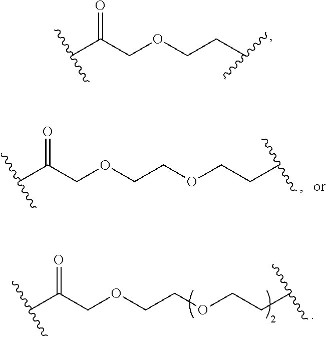

In some embodiments, L¹ is

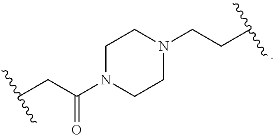 [SMILES: *CCN1CCN(C(=O)C*)CC1]

In some embodiments L¹ is

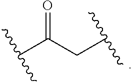 [SMILES: *CC(*)=O]

In some embodiments, a compound of Formula (I) is

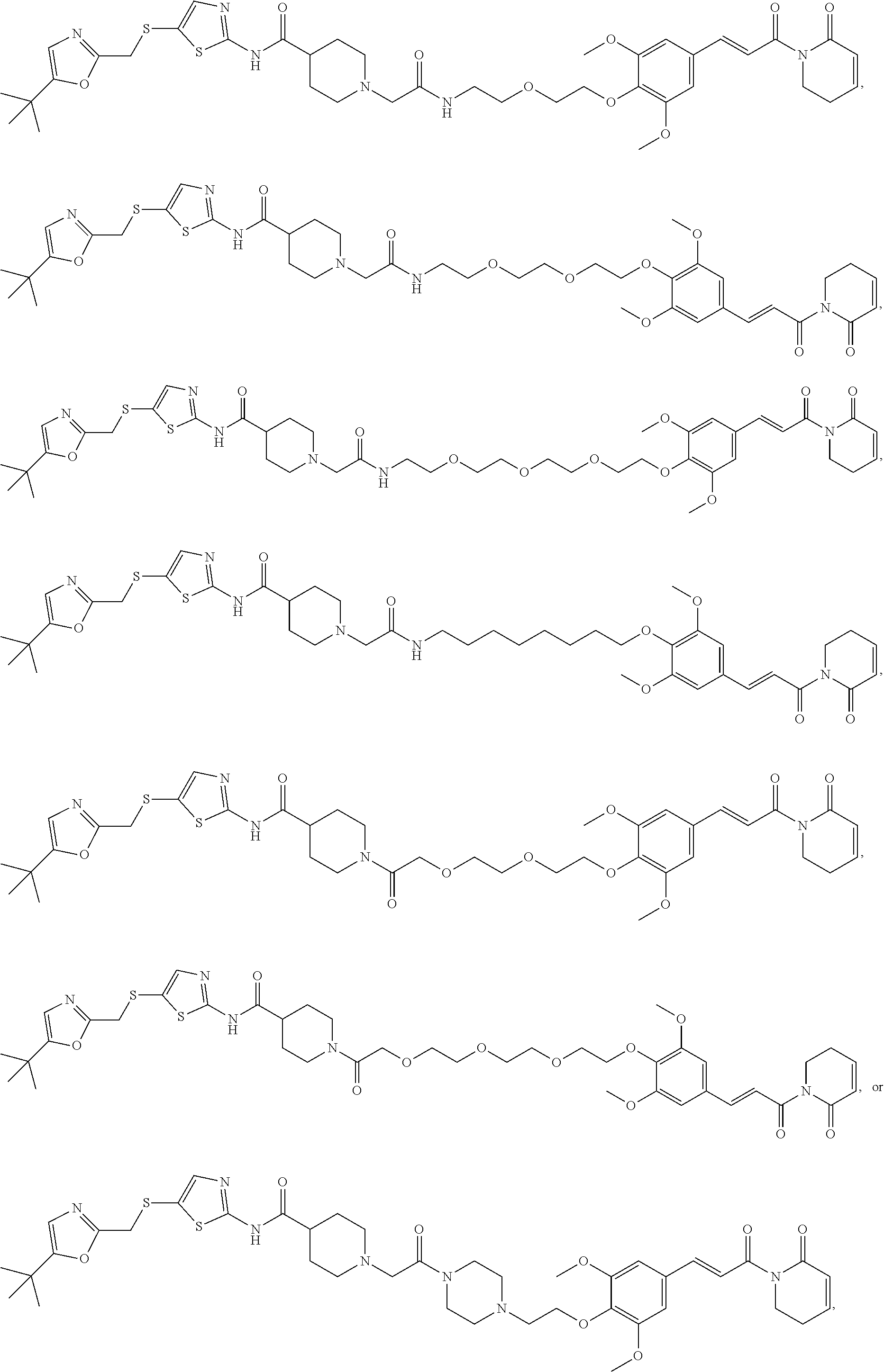

or a pharmaceutically acceptable salt, hydrate, solvate, tautomer, or prodrug thereof.

In some embodiments, a compound of Formula (I) is

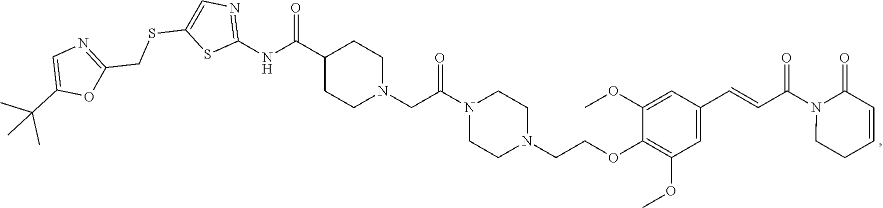 [SMILES: COc1cc(/C=C/C(=O)N2CCC=CC2=O)cc(OC)c1OCCN1CCN(C(=O)CN2CCC(C(=O)Nc3ncc(SCc4ncc(C(C)(C)C)o4)s3)CC2)CC1]

or a pharmaceutically acceptable salt, hydrate, solvate, tautomer, or prodrug thereof.

In some embodiments, a compound of Formula (I) is

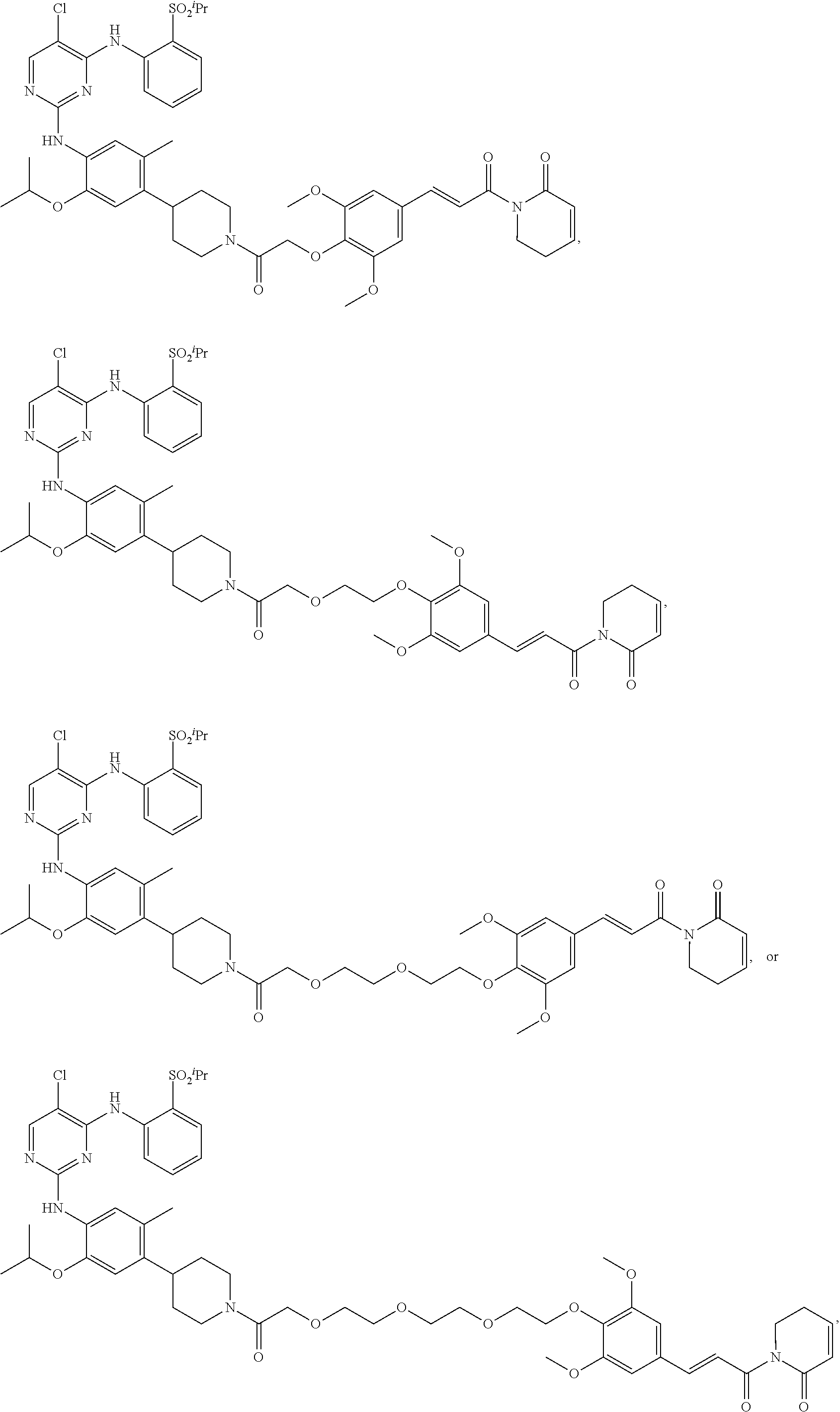

or a pharmaceutically acceptable salt, hydrate, solvate, tautomer, or prodrug thereof.

In some embodiments, a compound of Formula (I) is

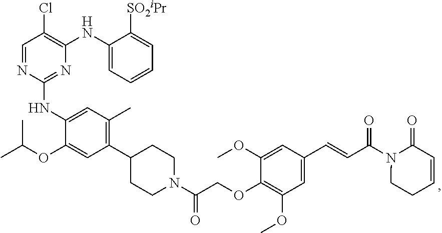

or a pharmaceutically acceptable salt, hydrate, solvate, tautomer, or prodrug thereof.

Compounds of the disclosure can be made or modified by means known in the art of organic synthesis. Methods for optimizing reaction conditions, if necessary minimizing competing by-products, are known in the art. Additional reaction schemes, optimization, scale-up, and protocols may be determined by the skilled artesian by use of commercially available structure-searchable database software, for instance, SciFinder® (CAS division of the American Chemical Society) and CrossFire Beilstein® (Elsevier MDL). For example, compounds of formulae herein can be made using methodology known in the art, including March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

For example, using the aforementioned methodology, the Formula (I) compounds can be synthesized by coupling of the “A” moiety with the L¹ moiety, then that piece attached to the appropriate PL (or PL derivative) moiety. Alternatively, the Formula (I) compounds can be synthesized by coupling the appropriate PL derivative moiety to the L¹ moiety, then that piece attached to the appropriate “A” moiety. Alternatively, the Formula (I) compounds can be synthesized by coupling the appropriate “A” moiety to a partial L¹ moiety, coupling the appropriate PL (or PL derivative) moiety to a second partial L¹ moiety, then coupling the two respective pieces via connection of the two partial L¹ moieties.

### Pharmaceutical Compositions and Administration

Provided herein is a pharmaceutical composition comprising a compound provided herein (e.g., a compound of Formula (I)), or a pharmaceutically acceptable salt, hydrate, solvate, tautomer, or prodrug thereof, and a pharmaceutically acceptable excipient.

In certain embodiments, the compound described herein is provided in an effective amount in the pharmaceutical composition.

In certain embodiments, the effective amount is a therapeutically effective amount. In certain embodiments, a therapeutically effective amount is an amount sufficient for treating a disease or disorder (e.g., cancer). In certain embodiments, a therapeutically effective amount is an amount sufficient for degrading a kinase (e.g., a CDK (e.g., CDK9 or CDK10) or anaplastic lymphoma kinase). In certain embodiments, a therapeutically effective amount is an amount sufficient for degrading a kinase (e.g., a CDK (e.g., CDK9 or CDK10) or anaplastic lymphoma kinase) and treating a disease or disorder (e.g., cancer). In certain embodiments, a therapeutically effective amount is an amount sufficient for inhibiting a kinase (e.g., a CDK (e.g., CDK9 or CDK10) or anaplastic lymphoma kinase). In certain embodiments, a therapeutically effective amount is an amount sufficient for inhibiting a kinase (e.g., a CDK (e.g., CDK9 or CDK10) or anaplastic lymphoma kinase) and treating a disease or disorder (e.g., cancer).

In certain embodiments, the effective amount is a prophylactically effective amount. In certain embodiments, a prophylactically effective amount is an amount sufficient for preventing a disease or disorder (e.g., cancer). In certain embodiments, a prophylactically effective amount is an amount sufficient for degrading a kinase (e.g., a CDK (e.g., CDK9 or CDK10) or anaplastic lymphoma kinase). In certain embodiments, a prophylactically effective amount is an amount sufficient for degrading a kinase (e.g., a CDK (e.g., CDK9 or CDK10) or anaplastic lymphoma kinase) and treating a disease or disorder (e.g., cancer). In certain embodiments, a prophylactically effective amount is an amount sufficient for inhibiting a kinase (e.g., a CDK (e.g., CDK9 or CDK10) or anaplastic lymphoma kinase). In certain embodiments, a prophylactically effective amount is an amount sufficient for inhibiting a kinase (e.g., a CDK (e.g., CDK9 or CDK10) or anaplastic lymphoma kinase) and treating a disease or disorder (e.g., cancer).

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj© 45), polyoxyethylene hydrogenated castor oil, polyethoxylated castor oil, polyoxymethylene stearate, and Solutol*), sucrose fatty acid esters, polyethylene glycol fatty acid esters (e.g., Cremophor®), polyoxyethylene ethers, (e.g., polyoxyethylene lauryl ether (Brij© 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

In certain embodiments, the compound or composition further comprises one or more additional agents. In some embodiments, the compound or composition is administered concurrently with, prior to, or subsequent to one or more additional pharmaceutical agents. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In some embodiments, the additional agent is an anti-proliferative agent or anti-cancer agent. In some embodiments, the additional agent is an anti-proliferative agent. In some embodiments, the additional agent is an anti-cancer agent.

In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

### Kits

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease or disorder (e.g., cancer) in a subject in need thereof. In certain embodiments, the kits are useful for preventing a disease or disorder (e.g., cancer) in a subject in need thereof. In certain embodiments, the kits are useful for reducing the risk of developing a disease or disorder (e.g., cancer) in a subject in need thereof.

In certain embodiments, a kit described herein further includes instructions for using the kit. In some embodiments, a kit disclosed herein includes information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease or disorder (e.g., cancer) in a subject in need thereof. In certain embodiments, the kits and instructions provide for reducing the risk of developing a disease or disorder (e.g., cancer) in a subject in need thereof. A kit described herein may include one or more additional pharmaceutical agents described herein as a separate composition.

### Methods of Use

Provided herein is a method of preventing or treating a disease or disorder in a subject in need thereof, the method comprising administering an effective amount of a compound of Formula (I), or a pharmaceutically acceptable salt, hydrate, solvate, tautomer, or prodrug thereof, or a pharmaceutical composition thereof.

Also provided herein is a method of preventing or treating a subject suffering from or susceptible to a disease or disorder, the method comprising administering an effective amount of a compound of Formula (I), or a pharmaceutically acceptable salt, hydrate, solvate, tautomer, or prodrug thereof, or a pharmaceutical composition thereof.

In certain embodiments, the method is a method of treating the disease or disorder or the subject.

Any of the methods provided herein may further comprise degrading or inhibiting a kinase.

Also provided herein is a use of a compound of Formula (I), or a pharmaceutically acceptable salt, hydrate, solvate, tautomer, or prodrug thereof, or a pharmaceutical composition thereof for the preparation of a medicament for preventing or treating a disease or disorder in a subject in need thereof.

Also provided herein is a use of a compound of Formula (I), or a pharmaceutically acceptable salt, hydrate, solvate, tautomer, or prodrug thereof, or a pharmaceutical composition thereof for the preparation of a medicament for preventing or treating a disease or disorder in a subject suffering from or susceptible to a disease or disorder.

Also provided herein is a compound of Formula (I), or a pharmaceutically acceptable salt, hydrate, solvate, tautomer, or prodrug thereof, or a pharmaceutical composition thereof for use in preventing or treating a disease or disorder in a subject in need thereof.

Also provided herein is a compound of Formula (I), or a pharmaceutically acceptable salt, hydrate, solvate, tautomer, or prodrug thereof, or a pharmaceutical composition thereof for use in preventing or treating a disease or disorder in a subject suffering from or susceptible to a disease or disorder.

Also provided herein is a method of inhibiting a kinase, the method comprising contacting a kinase with an effective amount of a compound of Formula (I), or a pharmaceutically acceptable salt, hydrate, solvate, tautomer, or prodrug thereof.

Also provided herein is a method of degrading a kinase, the method comprising contacting a kinase with an effective amount of a compound of Formula (I), or a pharmaceutically acceptable salt, hydrate, solvate, tautomer, or prodrug thereof.

In some embodiments, the contacting is in vitro or in vivo. In certain embodiments, the contacting is in vitro. In certain embodiments, in vitro methods provided herein can be carried out, e.g., in an assay, cell culture, or biological sample. In some embodiments, the contacting is in vivo, e.g., in an organism. In some embodiments, any of the compounds or compositions described herein are contacted with a cell ex vivo, meaning the cell is removed from an organism prior to the contacting. As will be evident to one of skill in the art, the term cell may be used to refer to a single cell as well as a population of cells.

In some embodiments, the inhibiting or degrading is achieved in MOLT4 cells, 293T cells, K562 cells, LNCap cells, 22RV1 cells, PC3 cells, DU145 cells, or NCI-H2228 cells. As will be evident to one of skill in the art, the term cell may be used to refer to a single cell as well as a population of cells.

In some embodiments, the methods further comprise measuring or assessing the level of one or more properties of the cell. In some embodiments, the level of one or more properties of the cell is assessed following contacting the cell with any of the compounds or compositions described herein. In some embodiments, the level of one or more properties following contacting the cell with any of the compounds or compositions described herein is compared to the level of one or more properties in a reference sample or prior to contacting the cell with the compounds or composition. In some embodiments, the contacting the cell with any of the compounds or compositions described herein increases one or more properties of the cell. In some aspects, the methods described herein may be used to determine whether a cell is susceptible to treatment with the compounds or compositions described herein. In some embodiments, if the level of one or more properties is increased following contacting the cell with any of the compounds or compositions described herein, the cell is determined to be susceptible to treatment with the compound or composition. In some embodiments, if the level of one or more properties is increased following contacting the cell with any of the compounds or compositions described herein, the compound or composition is determined to be a candidate for a disease or disorder associated with the cell.

In some embodiments, the method comprises administering the compound to a subject. In some embodiments, the subject is identified as being in need of treatment. In certain embodiments the subject is suffering from a disease or disorder (e.g., cancer). In some embodiments, the subject is diagnosed with a disease or disorder (e.g., cancer). In some embodiments, the subject is susceptible to a disease or disorder (e.g., cancer).

In certain embodiments, the subject is an animal. The animal may be of either sex and may be at any stage of development. In certain embodiments, the subject described herein is a human. In certain embodiments, the subject is a human aged less than 1 month. In certain embodiments, the subject is a human aged 1 month to less than 2 years. In certain embodiments, the subject is a human aged 2 to less than 12 years. In certain embodiments, the subject is a human aged 12 to less than 17 years. In certain embodiments, the subject is a human aged 17 or above. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In some embodiments, the disease or disorder is associated with a CDK. In some embodiments, the disease or disorder is associated with increased activity and/or increased production of a CDK. In some embodiments, the CDK is CDK9 or CDK10. In some embodiments, the disease or disorder is associated with anaplastic lymphoma kinase. In some embodiments, the disease or disorder is associated with increased activity and/or increased production of anaplastic lymphoma kinase.

In some embodiments, the disease is a proliferative disease. In some embodiments, the disease is cancer. In some embodiments, the cancer is a cancer that expresses KEAP1. In some embodiments, the cancer is a leukemia that expresses KEAP1. In some embodiments, the cancer is a solid tumor or liquid tumor. In some embodiments, the cancer is a solid tumor. In some embodiments, the cancer is a liquid tumor. In some embodiments, the cancer is lung cancer. In some embodiments, the cancer is non-small cell lung cancer, prostate cancer, acute lymphoblastic leukemia, or chronic myelogenous leukemia. In some embodiments, the cancer is non-small cell lung cancer. In some embodiments, the non-small cell lung cancer is ALK-positive non-small cell lung cancer. In some embodiments, the cancer is prostate cancer. In some embodiments, the prostate cancer is AR-positive. In some embodiments, the prostate cancer is AR-negative. In some embodiments, the cancer is bone cancer, brain cancer, breast cancer, cervical cancer, colon cancer, esophageal cancer, head and neck cancer, kidney cancer, liver cancer, melanoma, NUT carcinoma, ovarian cancer, pancreatic cancer, or uterus cancer. In some embodiments, the cancer is biliary tract cancer, bladder cancer, breast cancer, colorectal cancer, liver cancer, or stomach cancer. In some embodiments, the cancer is breast cancer, colorectal cancer, esophageal cancer, glioblastoma, inflammatory myofibroblastic tumor, kidney cancer, neuroblastoma, ovarian cancer, pancreatic cancer, rhabdomyosarcoma, salivary gland cancer, or thyroid cancer. In some embodiments, the cancer is a hematological malignancy. In some embodiments, the cancer is leukemia. In some embodiments, the cancer is acute lymphoblastic leukemia. In some embodiments, the cancer is T-cell acute lymphoblastic leukemia. In some embodiments, the cancer is chronic myelogenous leukemia. In some embodiments, the cancer is acute myeloid leukemia, chronic lymphocytic leukemia, or T-cell leukemia. In certain embodiments, the cancer is lymphoma. In some embodiments, the cancer is anaplastic large-cell lymphoma. In some embodiments, the cancer is ALK-positive anaplastic large cell lymphoma or primary cutaneous anaplastic large cell lymphoma. In certain embodiments, the cancer is diffuse large B-cell lymphoma, Burkitt's lymphoma, T-cell lymphoma, aggressive natural killer cell leukemia, Hodgkin's lymphoma, or mantle cell lymphoma. In certain embodiments, the cancer is multiple myeloma.

In certain embodiments, the compound inhibits up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of a kinase at a compound concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compound degrades up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of a kinase at a compound concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In some embodiments, the kinase is a kinase provided herein. In some embodiments, the kinase is a CDK. In certain embodiments, the kinase is CDK9, CDK10, or anaplastic lymphoma kinase inhibitor. In some embodiments, the kinase is CDK9 or CDK10. In certain embodiments, the kinase is CDK9. In some embodiments, the kinase is CDK10. In some embodiments, the kinase is anaplastic lymphoma kinase.

In some embodiments, the degrading is achieved by recruitment of a cullin ring-related ubiquitin E3 ligase. In some embodiments, the cullin ring-related ubiquitin E3 ligase is KEAP1.

### EXAMPLES

### Example 1: Identification of Piperlongumine (PL) as an E3 Ligase Ligand to Induce Targeted Protein Degradation

A proteolysis-targeting chimera (PROTAC) is a bifunctional molecule that can ubiquitinate and degrade target protein through recruiting E3 ligase. However, very limited numbers of E3 ligases can be used in the PROTAC field. Piperlongumine (PL) is a natural product with anti-cancer and anti-aging properties. Competitive activity-based protein profiling (ABPP) demonstrated that PL can recruit multiple E3 ligases, and may serve as a new E3 ligase ligand. A series of PL-SNS-032 conjugates were synthesized and evaluated. The compound 955 potently degraded CDK9 and CDK10 and exhibited higher potency against various tumor cells than SNS-032. Through TurboID-based proteomics and mechanistic studies, KEAP1 was identified as the functional E3 ligase. Next, a PL-Ceritinib conjugate was synthesized and confirmed ALK-fusion protein was also degraded. Collectively, these findings suggested that PL is a novel covalent E3 ligase ligand to generate potent anticancer PROTACs.

Previous studies have shown that PL is a selective senolytic agent and other studies have also demonstrated that it is a safe anti-tumor agent. PL contains two Michael acceptors and the previous studies showed that it can covalently bind to GSTP1 and GSTO1. Previous studies found that PL binds to 172 proteins in senescent lung fibroblast WI38 cells.

Here, PL was linked with SNS-032, a CDK9 inhibitor, and it was discovered that the PL-SNS-032 conjugates induced potent CDK9 degradation. Mechanistically, it was demonstrated that PL-SNS-032 conjugates degraded CDK9 in a ubiquitin-proteasome system (UPS) dependent manner. The TurboID-bait assay was developed to identify the functional E3 ligases. Furthermore, ALK-fusion protein was successfully degraded by a PL-based ALK PROTAC, indicating the great potential of PL as a new E3 ligase ligand.

### Identification of PL-Binding E3 Ligases

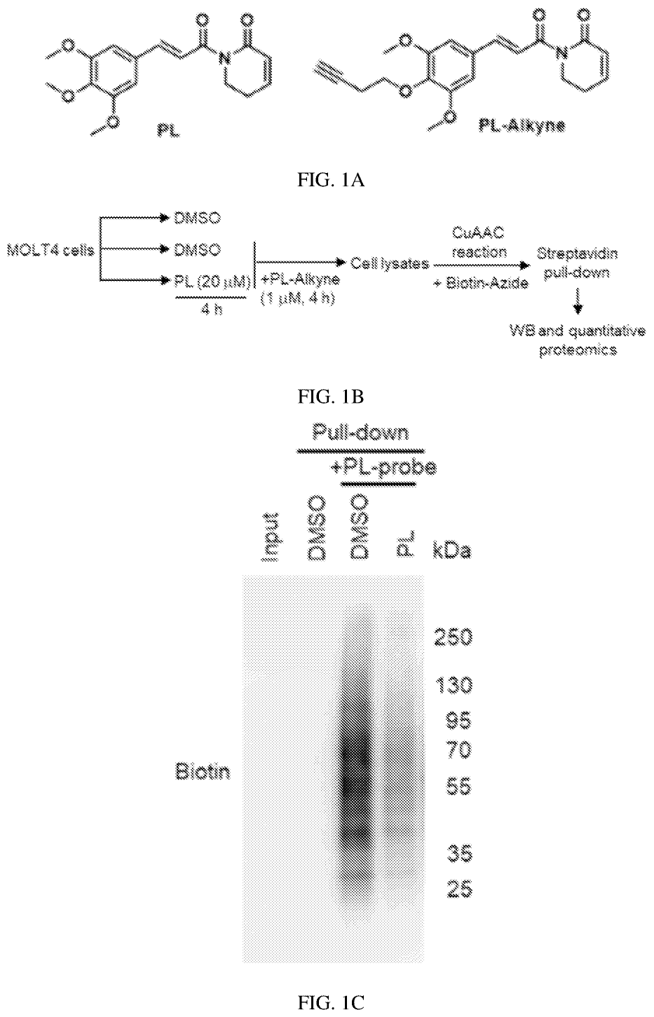

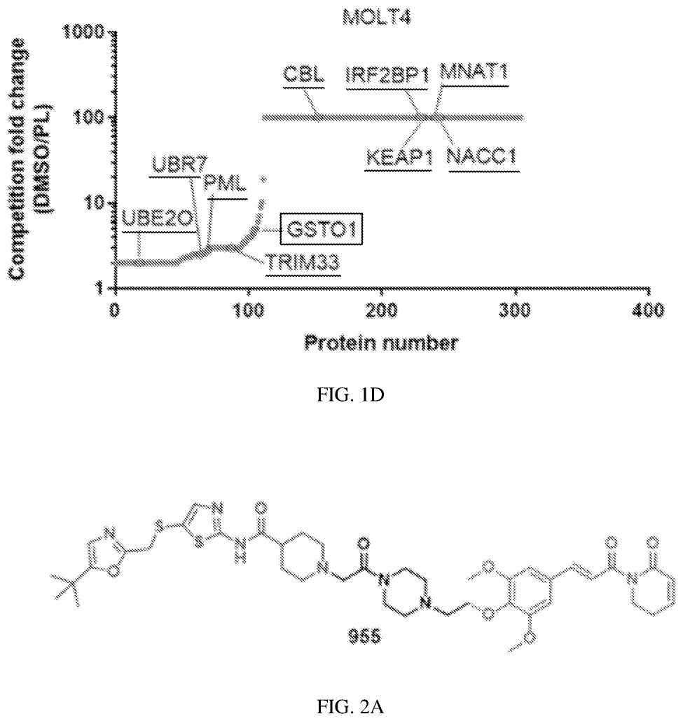

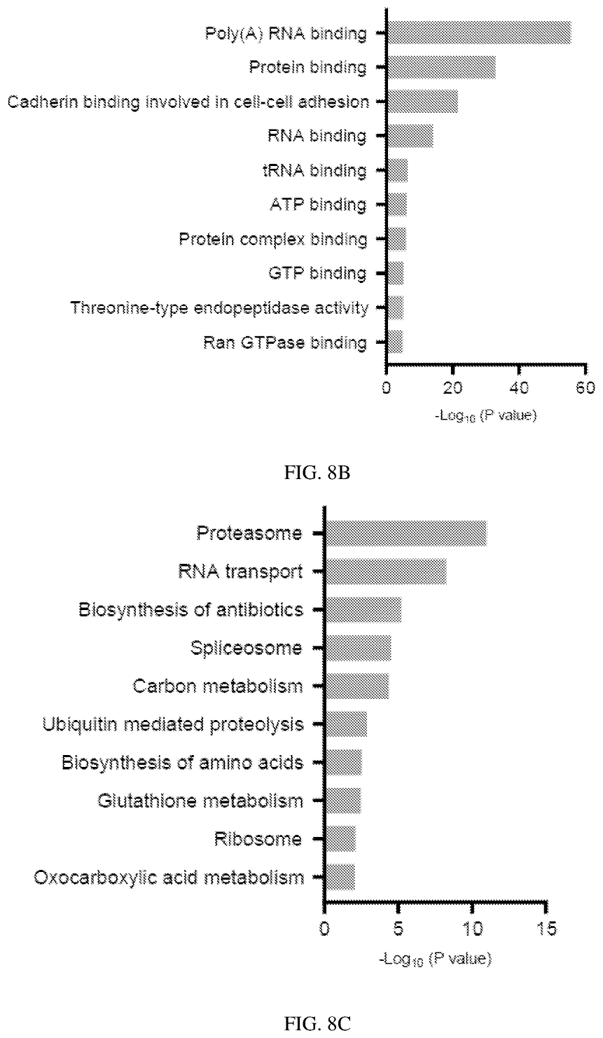

PL (FIG. 1A) binds to 172 target proteins in senescent fibroblast WI38 cells, including 8 E3 ligases. To characterize PL binding proteins, especially E3 ligases in cancer cells, a PL-Alkyne probe (FIG. 1A) was used to enrich the PL-binding proteins in MOLT4 (Human acute lymphoblastic leukemia, ALL) cells. After treatment, the cell lysates were extracted and used to perform a copper-catalysed azide-alkyne cycloaddition (CuAAC) reaction and then the PL-biotin-labeled proteins were pulled down by streptavidin beads. The proteins were eluted and detected by immunoblot or directly digested by trypsin on beads to identify them using LC-MS/MS (FIG. 1B). To further exclude the non-specific binding proteins, a group in which the cells were pretreated with high concentration of PL and then treated with the PL-Alkyne probe was also included (FIG. 1B). The western blot result showed that PL-probe pulled down many proteins and after PL competition, very faint bands were detected (FIG. 1C), which indicated that most of pull-downed proteins were PL-binding proteins. The mass spectrometry (MS) results showed that PL can recruit about 300 proteins (FIG. 1D). Gene ontology (GO) and Kyoto encyclopedia of genes and genomes (KEGG) pathway enrichment analyses was performed of those PL-binding proteins and it was found that the proteins involved in ubiquitin-protein ligase activity were highly enriched (FIGS. 8A to 8C), including 9 E3 ligases (FIG. 1D), which indicated PL also bound to multiple E3 in cancer cells. GSTO1 was also detected as a target of PL, which was a confirmed target of PL.

### Generation of PL-SNS-032 Conjugated Compounds

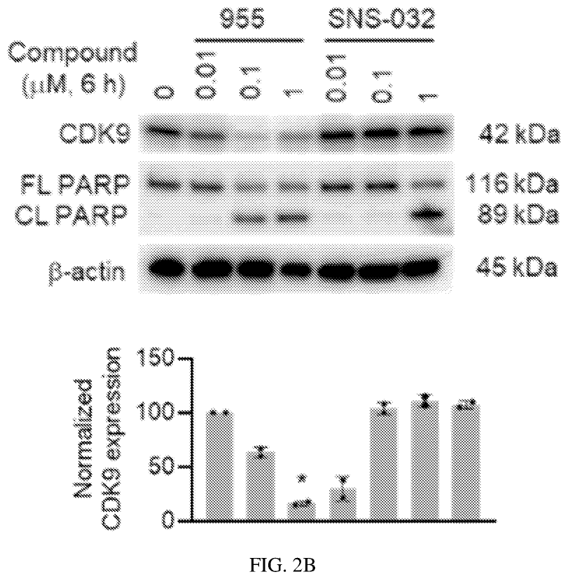

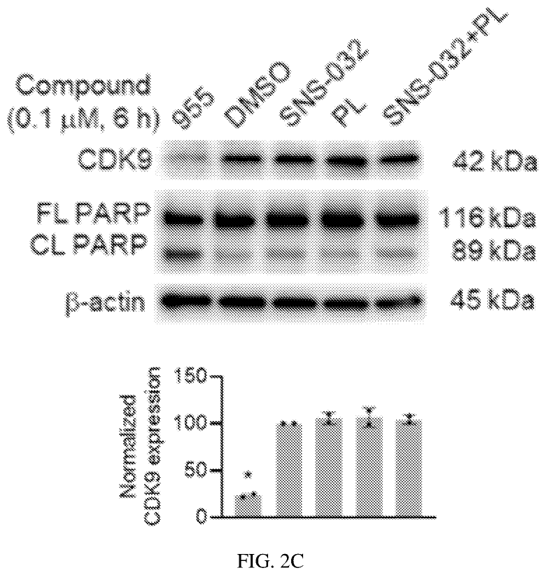

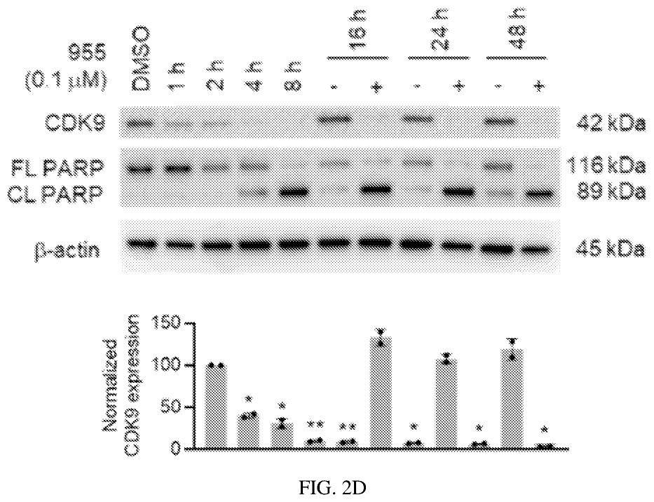

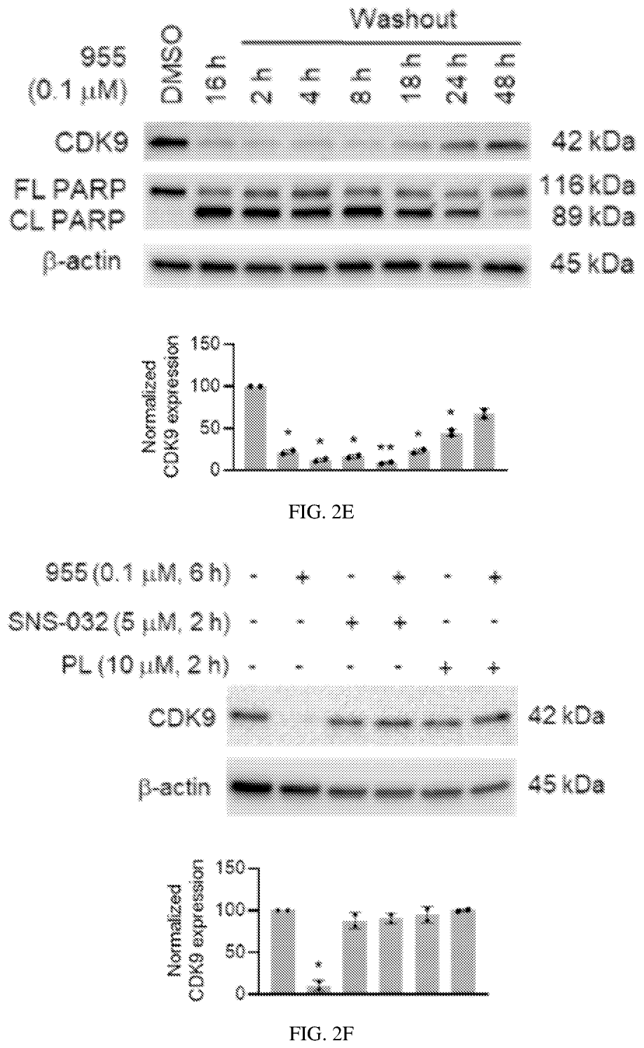

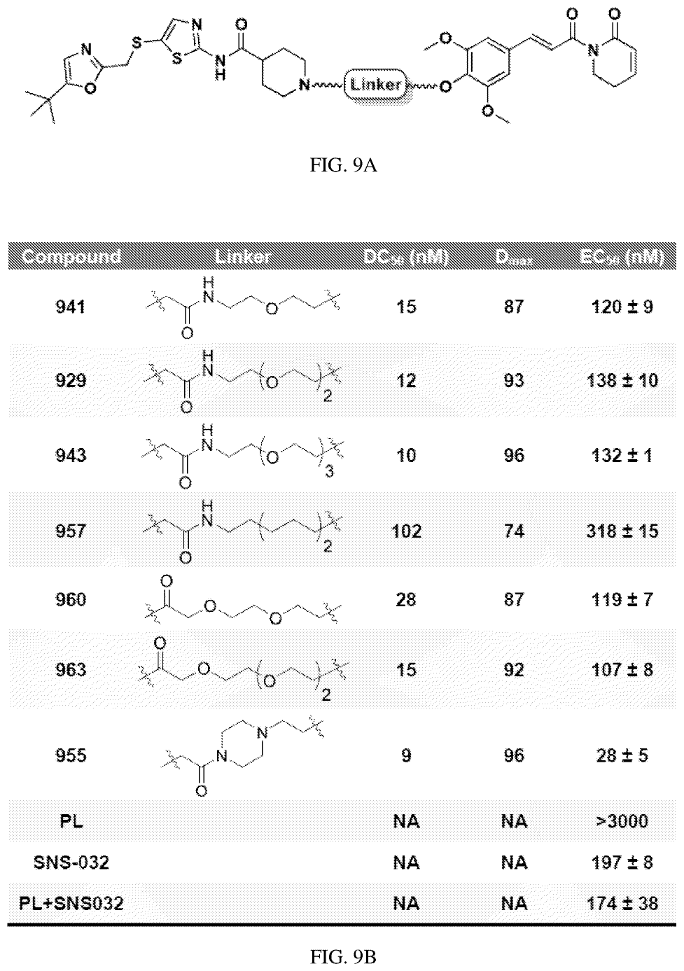

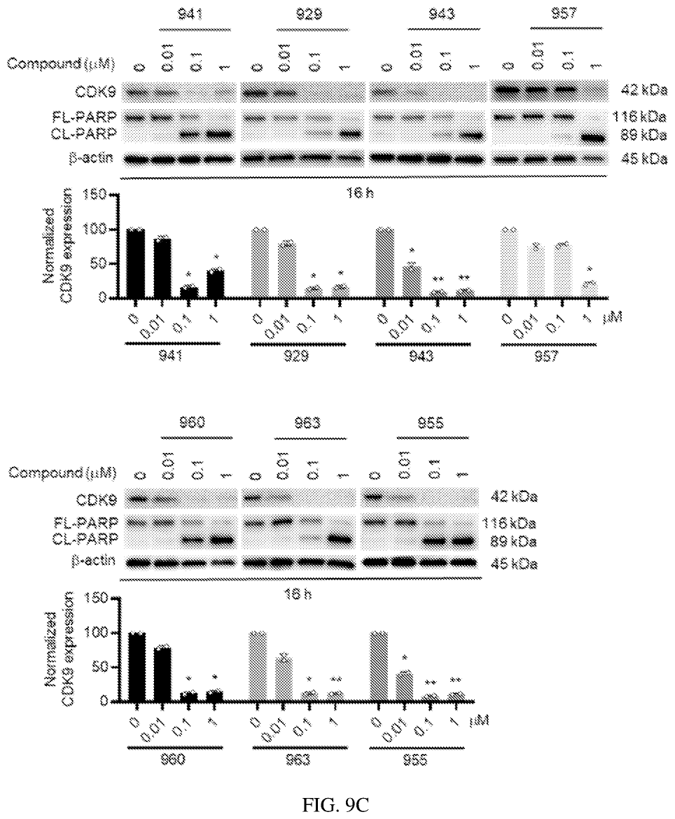

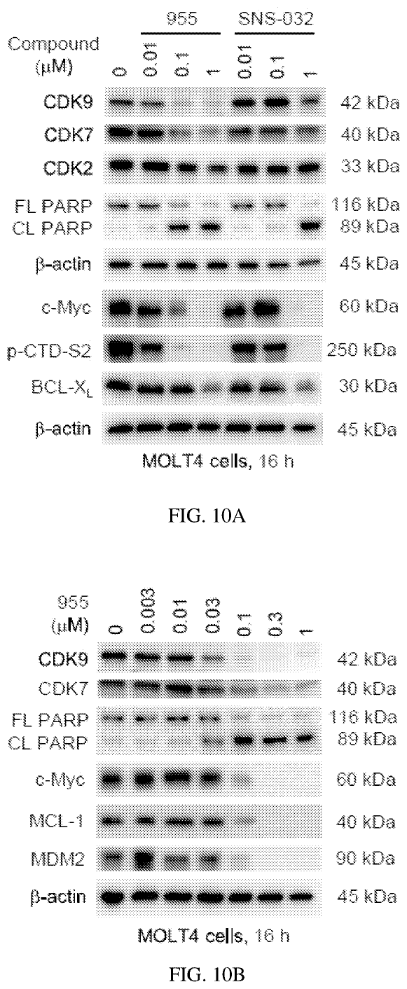

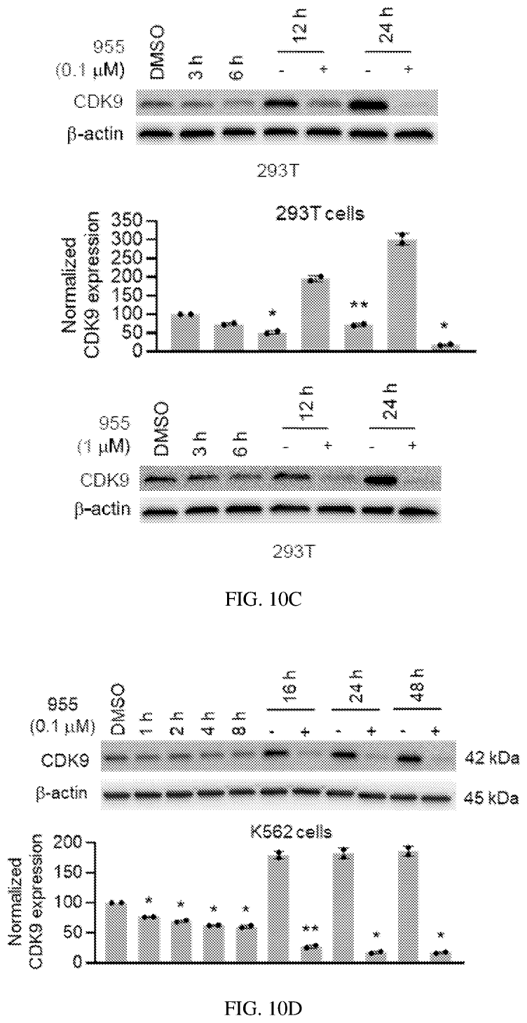

Following the competitive ABPP results, it was hypothesized that PL may be used as a covalent E3 ligase ligand to build PROTACs to degrade the POI. To prove this idea, SNS-032, a relative CDK9 selective inhibitor, was conjugated with PL to generate a series of PL-SNS-032 bifunctional molecules with linkers of different types and lengths (FIGS. 9A to 9B). CDK9 is well-documented target protein for cancer treatment, and a previous study had synthesized a thalidomide-SNS-032 conjugated compound to successfully induce CDK9 degradation. PARP cleavage and viability assays were used to evaluate those compounds in MOLT4 cells (FIGS. 9B and 9C) and the compound 955 was selected for further study (FIGS. 2A, 9B, and 9C). 955 induced the degradation of CDK9 with a DC₅₀ of 9 nM (FIG. 9B) after 16 hour treatment in MOLT4 cells. Even with short term treatment (6 hours), 955 still potently degraded CDK9, while the warhead SNS-032 showed no degradation effect (FIG. 2B). More significant up-regulation of cleaved PRAP under 955 treatment indicated that 955 triggered more apoptotic cell death compared with SNS-032 (FIG. 2B). To exclude the possibility that the degradation of CDK9 was caused by the combination/synergy effect of PL and SNS-032, MOLT4 cells were treated with either PL or SNS-032 alone or in combination, and the results showed that only 955, degraded CDK9 (FIG. 2C). In addition, the time-course experiment showed that CDK9 was completely degraded by 955 at 0.1 μM at 8 hours, and the effect lasted at least 48 hours (FIG. 2D). Similar results were also observed in 293T cells (FIG. 10A) and K562 cells (FIG. 10B). A further washout assay was performed, and it was observed that 955-induced CDK9 degradation could be fully recovered 48 hours after retrieving 955 (FIG. 2E). To further confirm that 955-induced CDK9 degradation depended on both PL and SNS-032, MOLT4 cells were pretreated with PL or SNS-032 and then treated by 955. The degradation of CDK9 induced by 955 was fully blocked by PL and SNS-032 (FIG. 2F).

### 955 is a Bona Fide CDK9 PROTAC Degrader

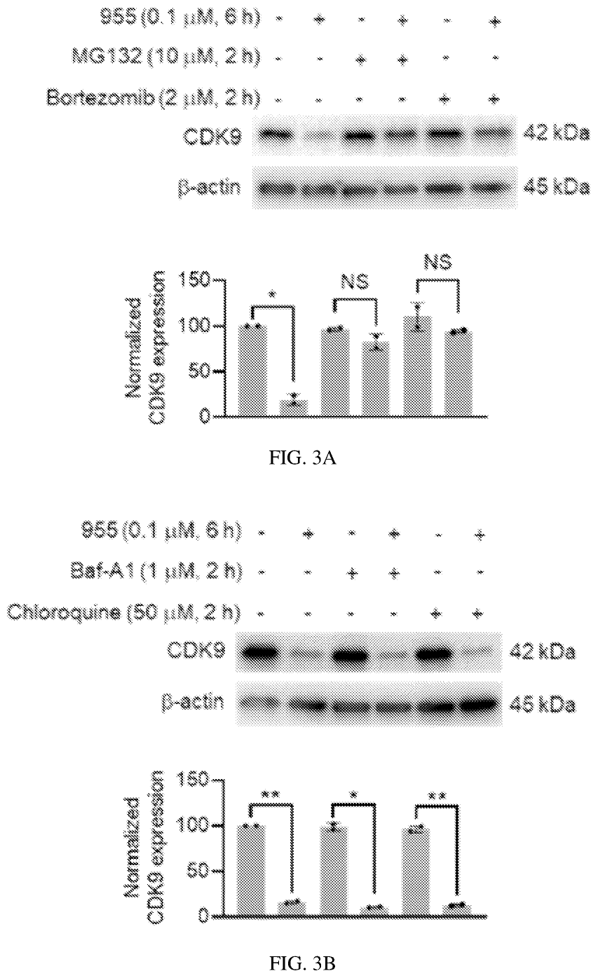

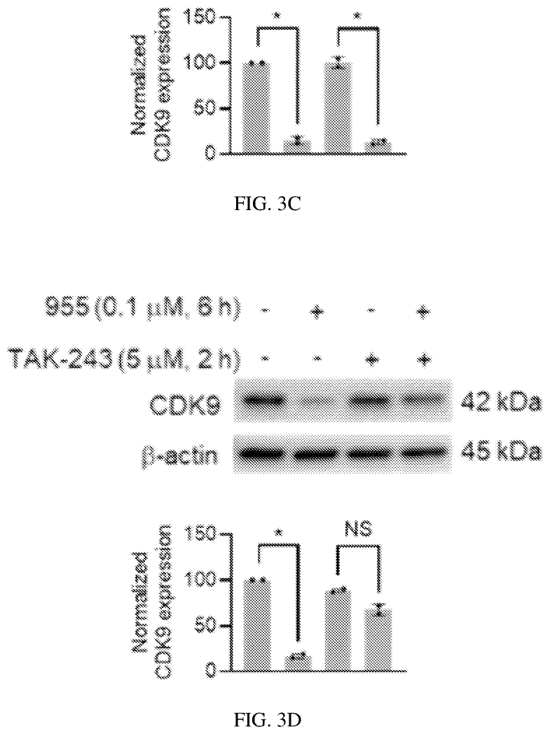

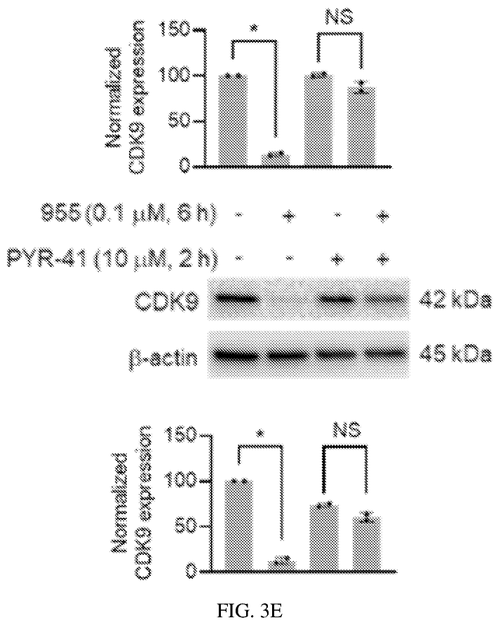

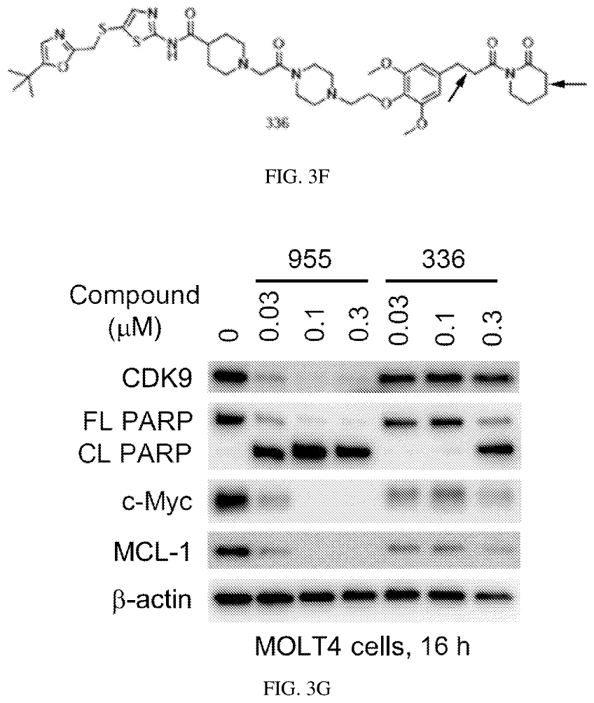

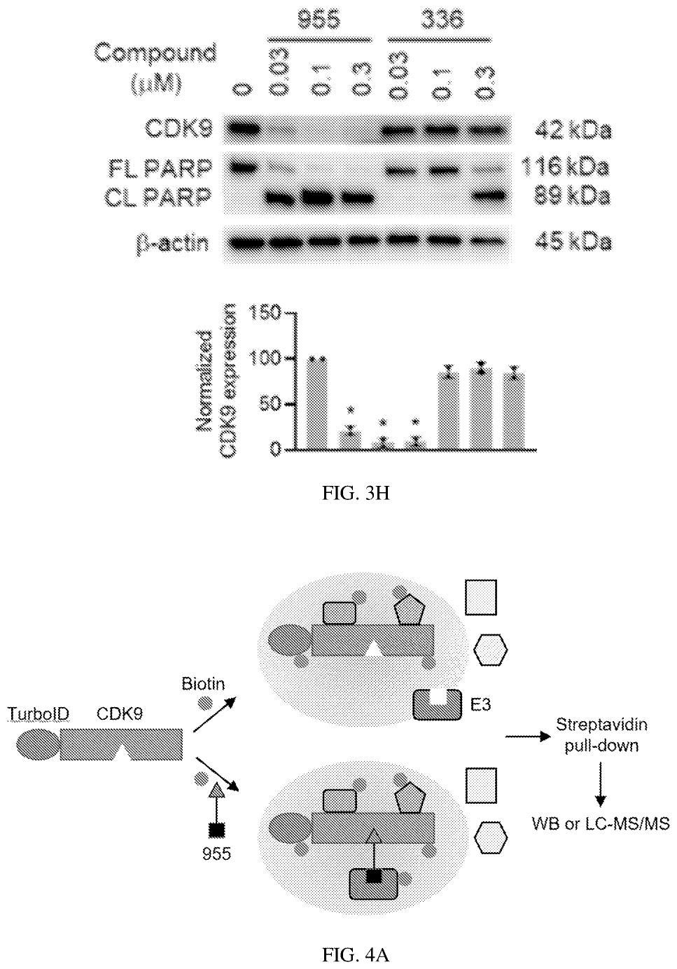

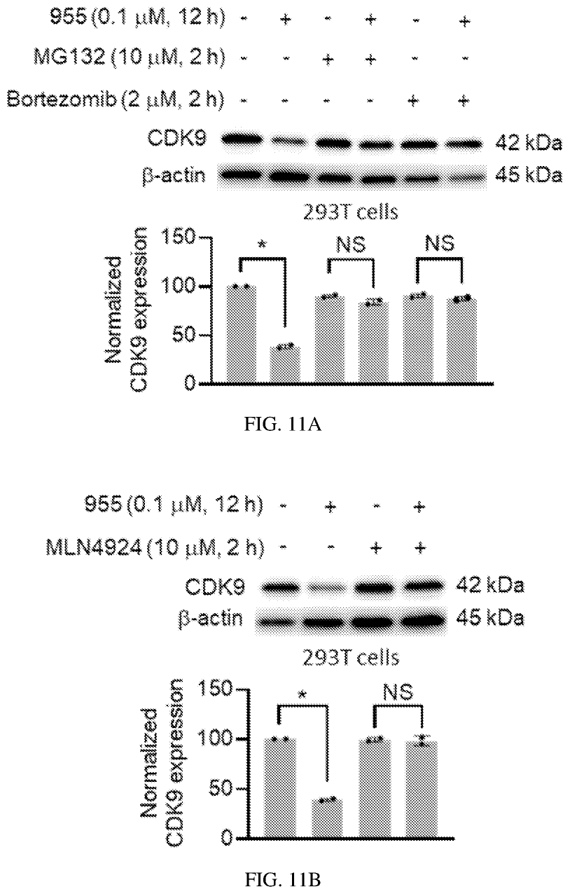

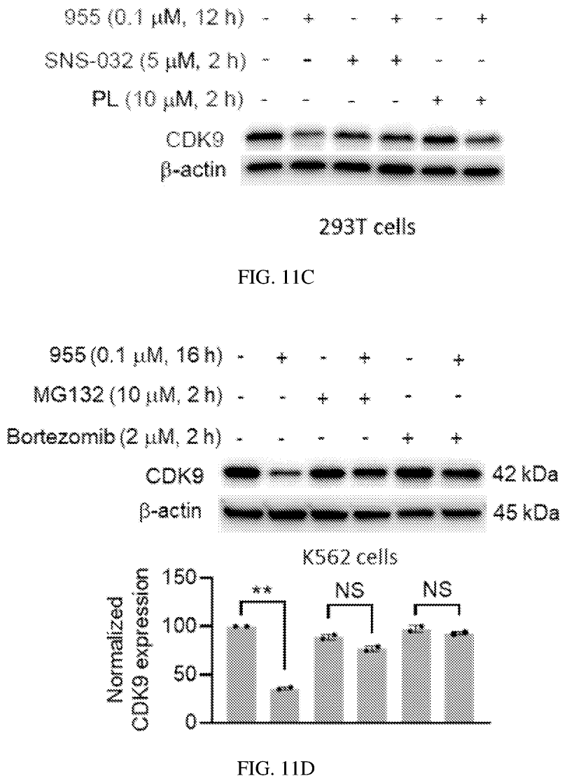

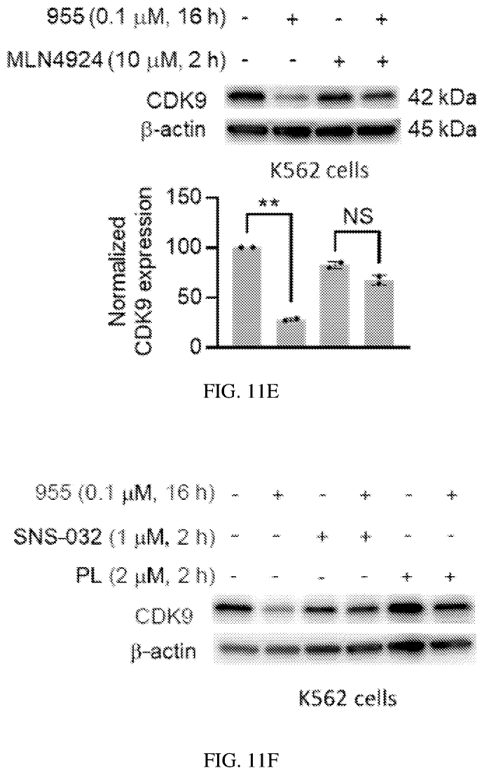

To further confirm the 955-induced CDK9 degradation was through the PROTAC effect, comprehensive inhibition assays of 955 were performed. First, MOLT4 cells were treated by 955 with or without proteasome inhibitor pretreatment. The results showed that the degradation of CDK9 could be blocked by two different proteasome inhibitors, MG132 or Bortezomib (FIG. 3A). Pretreatment by either of the two autophagy inhibitors, Baf-A1 and Chloroquine could not block CDK9 degradation (FIG. 3B). These assays demonstrated CDK9 degradation induced by 955 was through the proteasome. To rule out that the decrease of CDK9 was through cleavage of activated caspases under apoptosis, QVD, a pan-caspase inhibitor pretreatment, was performed and confirmed OVD could not block CDK9 degradation (FIG. 3C). Furthermore, two E1 inhibitors, PYR-41 and TAK-243, were used to verify that the degradation of CDK9 was E1-dependent (FIG. 3D). In the competitive ABPP assay, 9 PL-recruited E3 ligases were identified by the LC-MS/MS (FIG. 1D) Studies were performed to determine which type of E3 ligase(s) were involved in the process. MLN4924, a selective cullin ring-related ubiquitin E3 ligase(s) (CRLs) inhibitor through block the neddylation of cullin, was used to pretreat the cells. It was found that the 955-induced CDK9 degradation could be blocked (FIG. 3E), suggesting involvement. Similar assays were performed in 293T and K562 cells (FIG. 11), and it was observed that the degradation of CDK9 by 955 was also UPS-dependent. Accordingly, the functional E3 was a cullin or CRL. In addition, since PL has two Michael acceptors to bind to its targets covalently, control compound 336 was synthesized by saturating the two Michael acceptors (FIG. 3F). The Western blot assay confirmed that 336 could not induce CDK9 degradation (FIGS. 3G and 3H), which indicated that 955 covalently recruited CRL(s) via those Michael acceptors.

### Identification of E3 Ligase(s) Recruited by 955

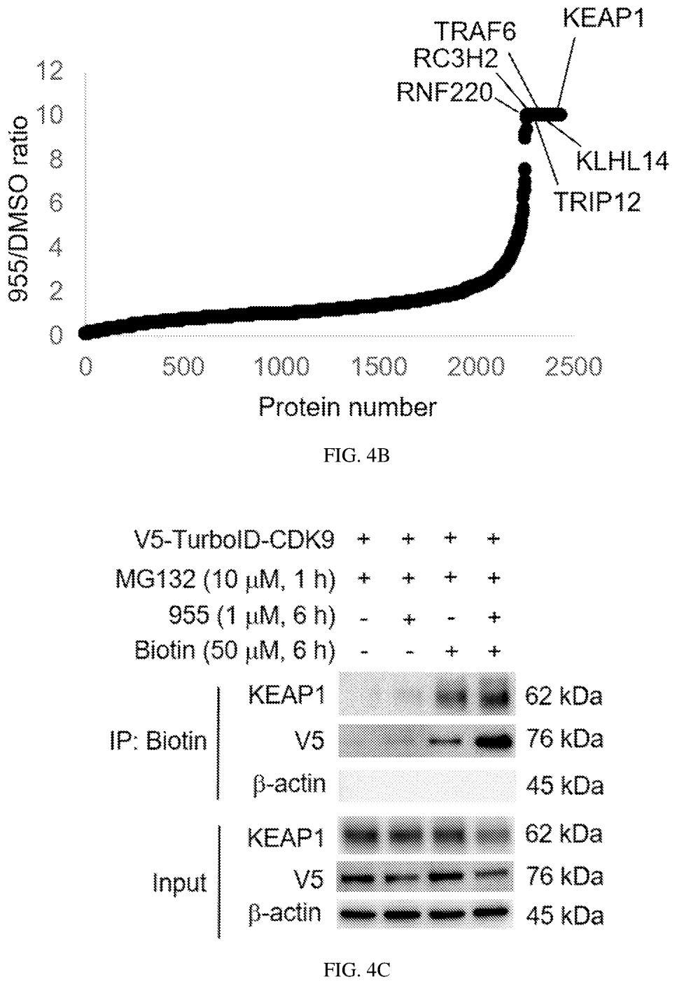

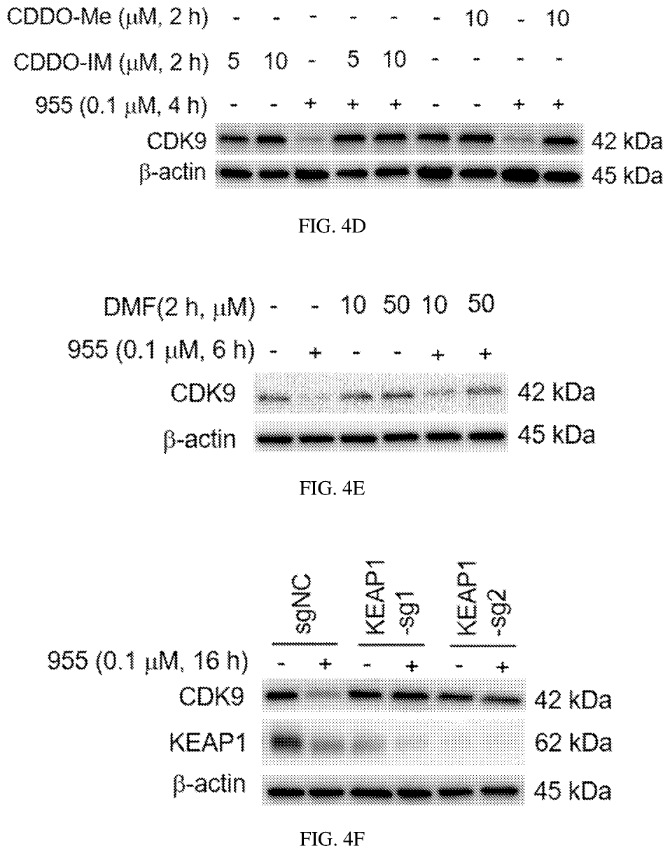

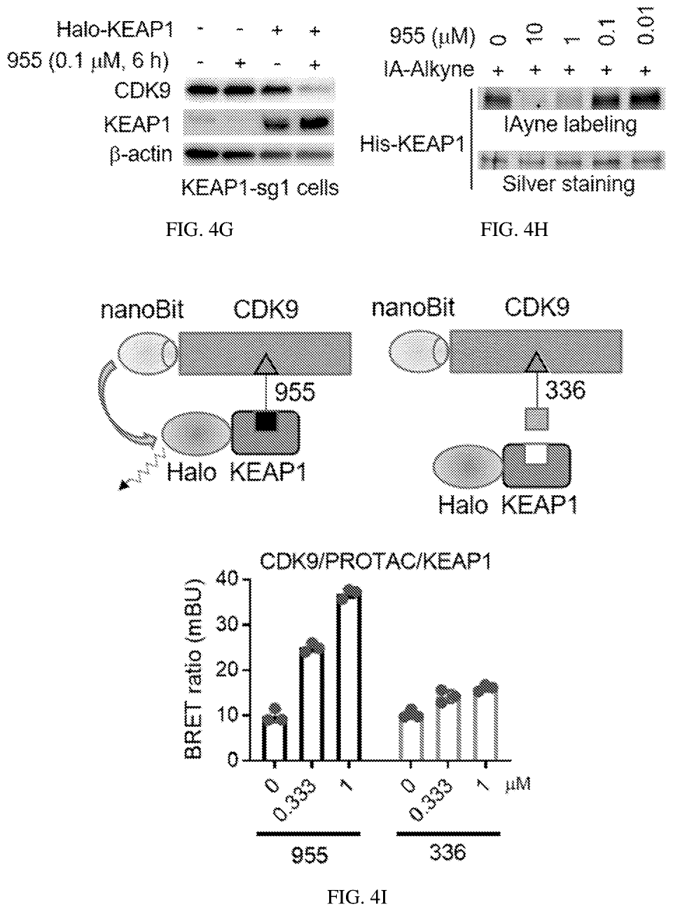

PROTACs can hijack an E3 ligase to catalyze the ubiquitination of target proteins. Because this process is dynamic, it is generally difficult to detect the target:E3 binding using the co-immunoprecipitation method. TurboID was used to detect weak and transient protein interaction through converting it to a stable covalent biotinyation. In the methods described herein, this technology was adapted to bait the E3 ligases recruited by 955 by overexpressing V5-TurboID-CDK9 in 293T cells and compare the biotinylated proteins with or without 955 treatment (FIG. 4A). The MS results showed that 955 hijacked 6 E3s (FIG. 4B). To narrow down the list for further mechanistic validation, the E3 identified by competitive ABPP (FIG. 1D) was overlapped with the TurboID-bait (FIG. 4B). It was observed that KEAP1 was the only CRL. A previous study had confirmed that a KEAP1-based peptide PROTAC could induce Tau protein degradation, indicating the potential of KEAP1 as a new E3 ligase for PROTAC design. To test if KEAP1 was the functional E3 ligase recruited by 955, Western blot was first used to confirm the results of TurboID-bait assay. Encouragingly, more KEAP1 could be pulled down under 955 treatment while other proteins such as and j-actin had no such effect (FIGS. 4C and 4J). To further validate that KEAP1 was covalently recruited by 955, MOLT4 cells were treated by 955 with or without each of KEAP1 inhibitors CDDO-ME, CDDO-IM and dimethyl fumarate (DMF). Inhibition of KEAP1 by CDDO-IM, CDDO-ME, or DMF completely blocked 955-mediated CDK9 degradation (FIGS. 4D to 4E). Forming a POI:PROTAC:E3 ternary complex is a necessary step for further ubiquitination and degradation. Therefore, to further test if 955 can induce the CDK9:955:KEAP1 ternary complex formation in living cells using nanoBRET assay, CDK9 was fused with HiBit-tag and KEAP1 with Halo-tag (FIG. 4I). The results demonstrated that 955 formed a stable ternary complex with CDK9 and KEAP1 in a dose-dependent manner, while the negative compound 336 did not, suggesting the formation of this ternary complex is Michael acceptor(s)-dependent (FIG. 4I). In conclusion, 955 can covalently hijack KEAP1 to mediate CDK9 degradation.

955 is a More Potent Anti-Cancer Agent than SNS-032

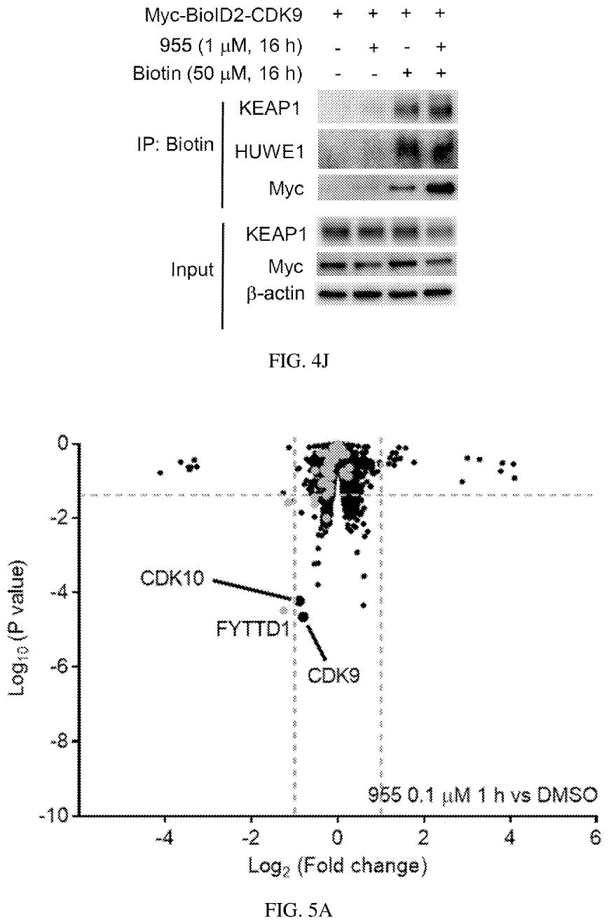

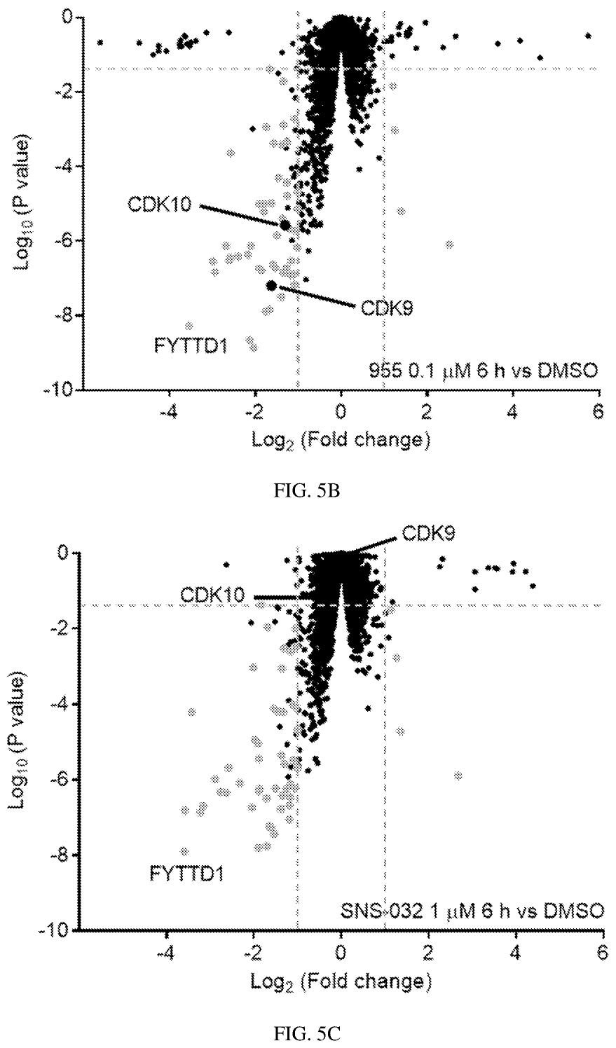

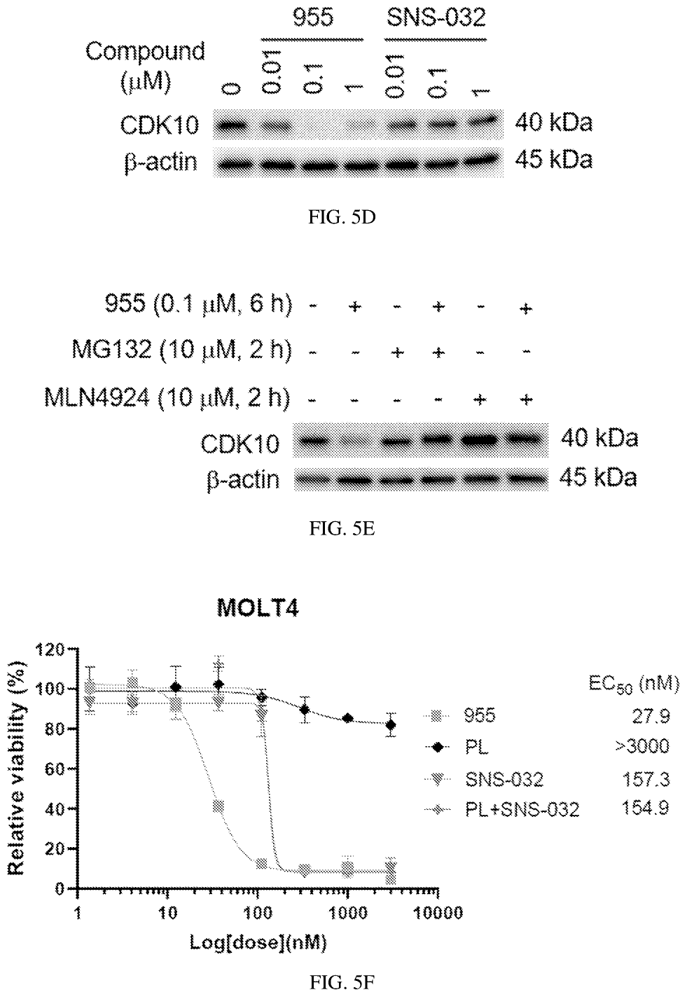

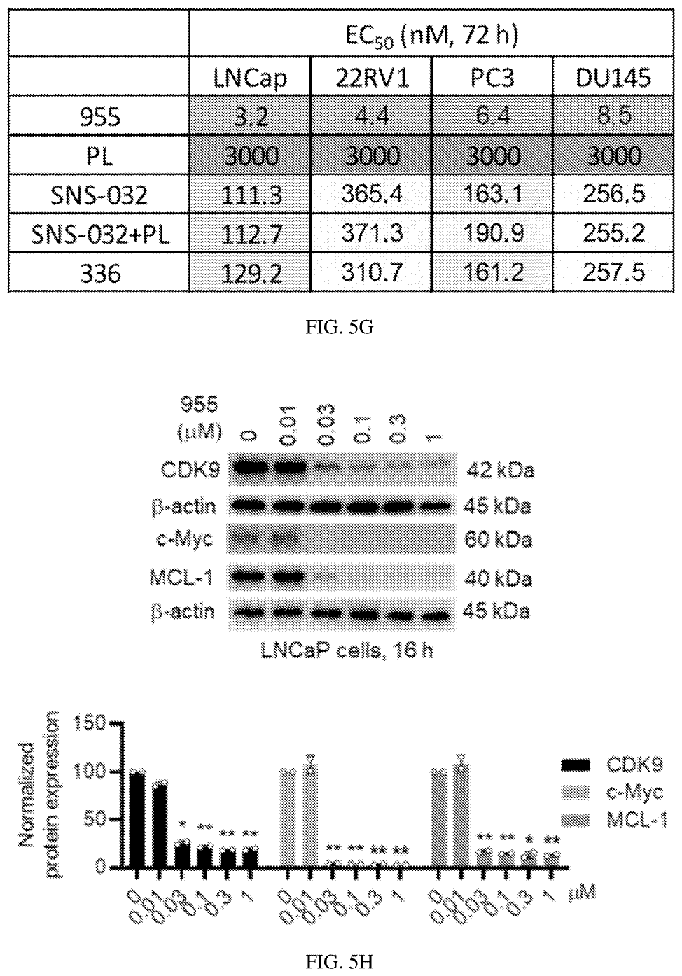

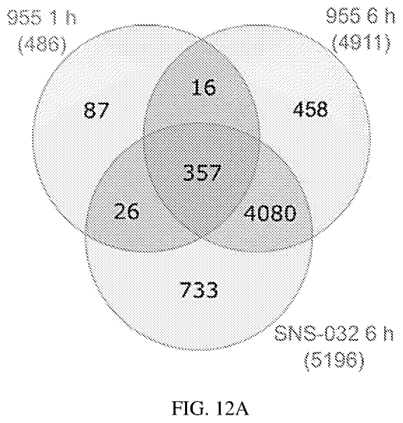

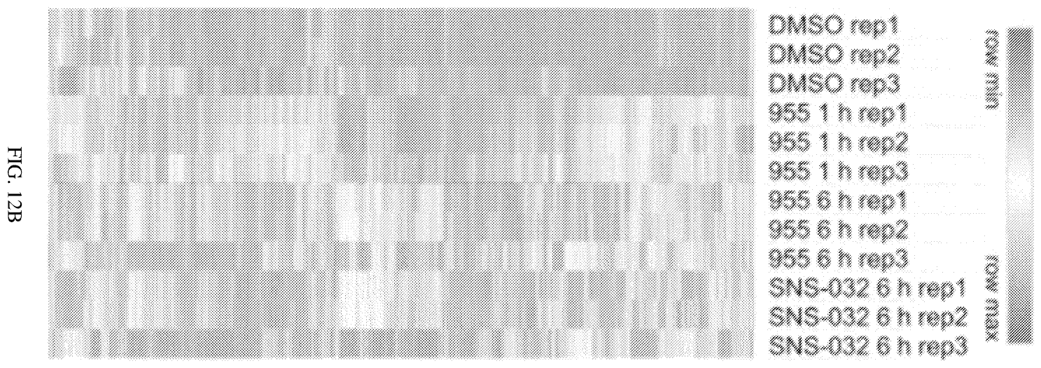

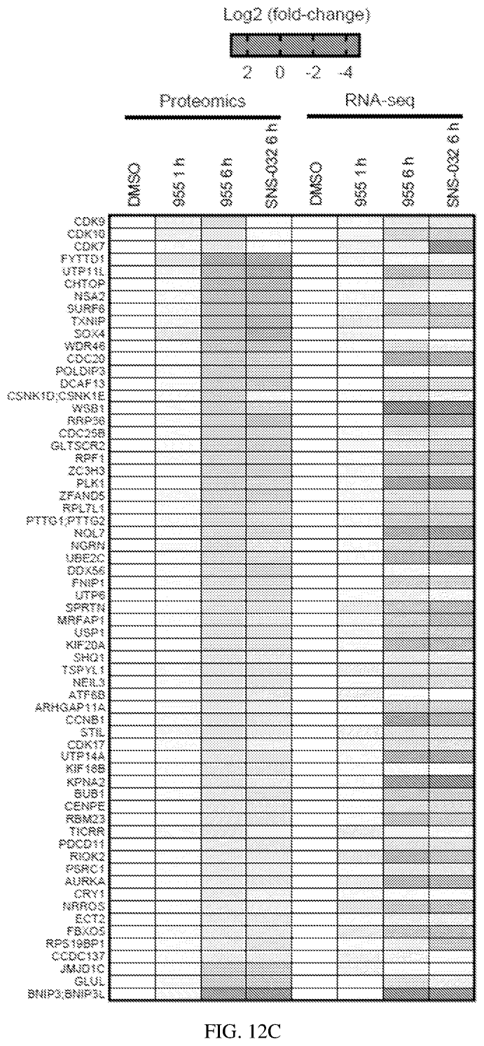

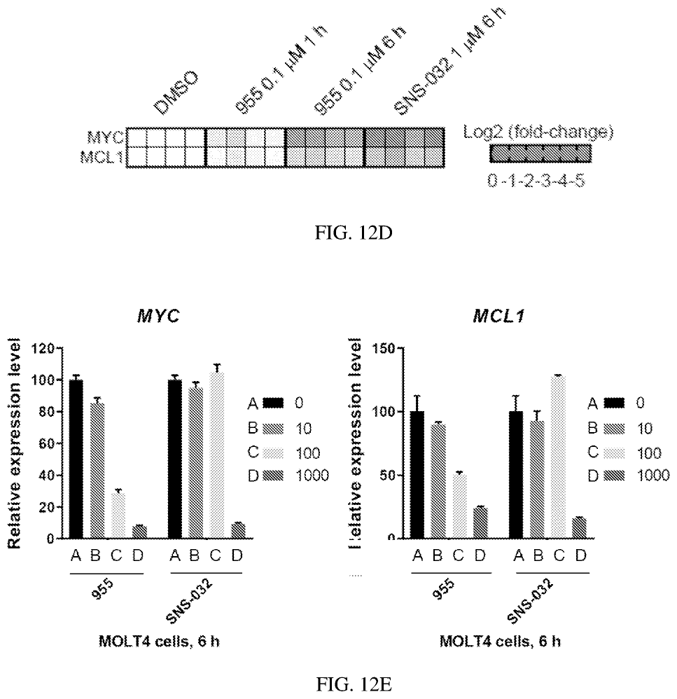

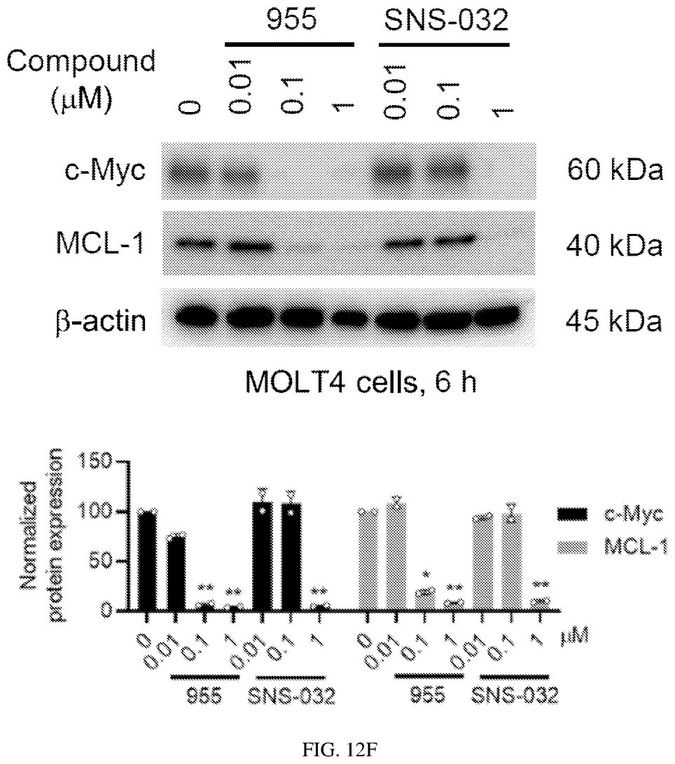

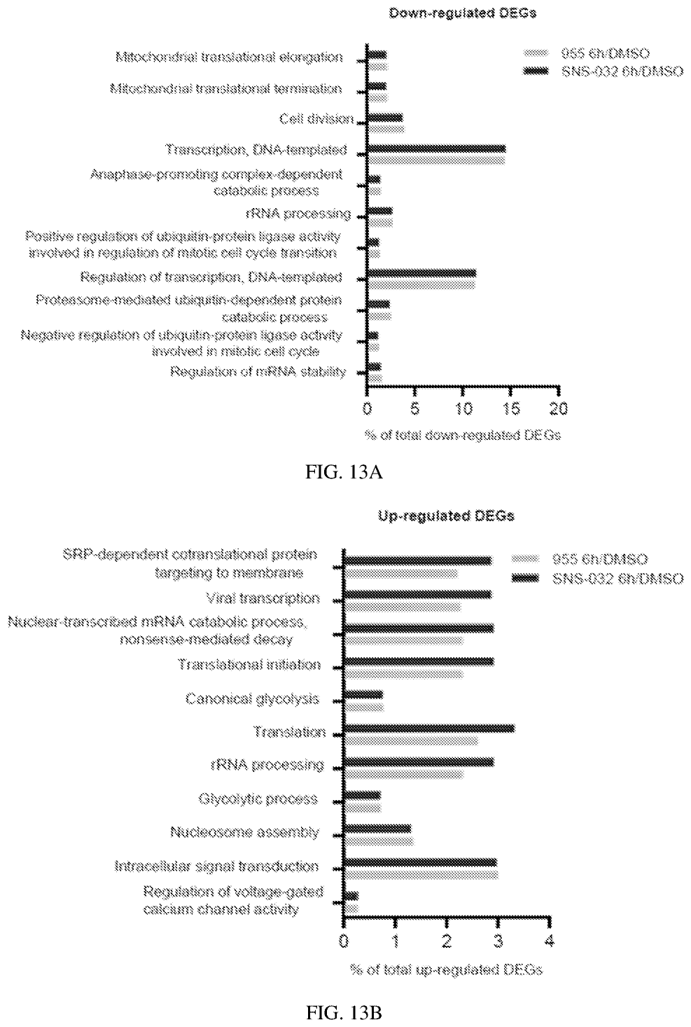

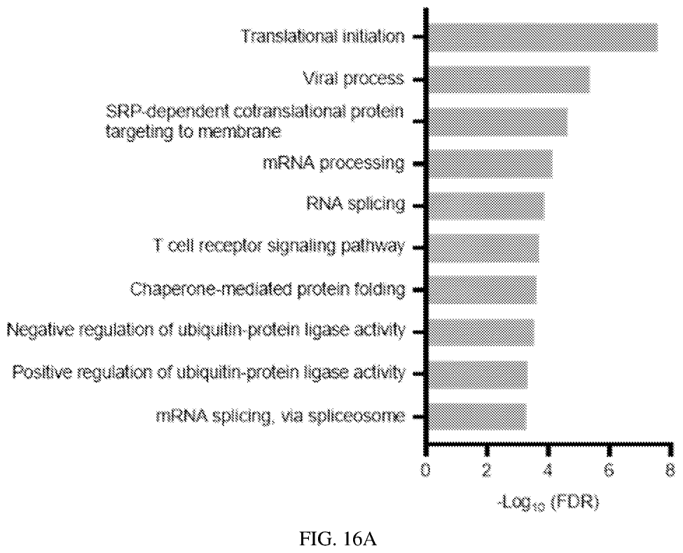

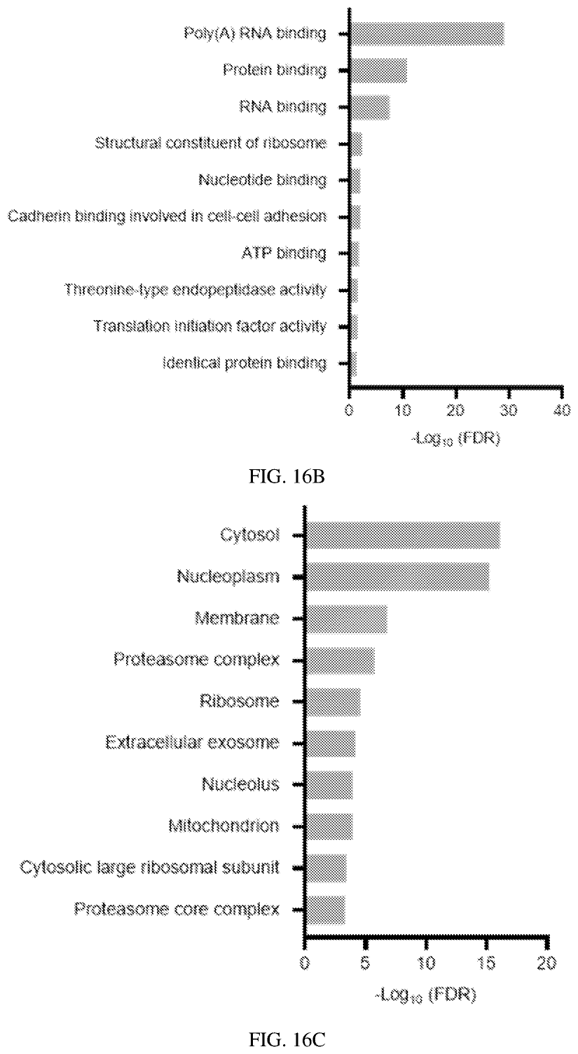

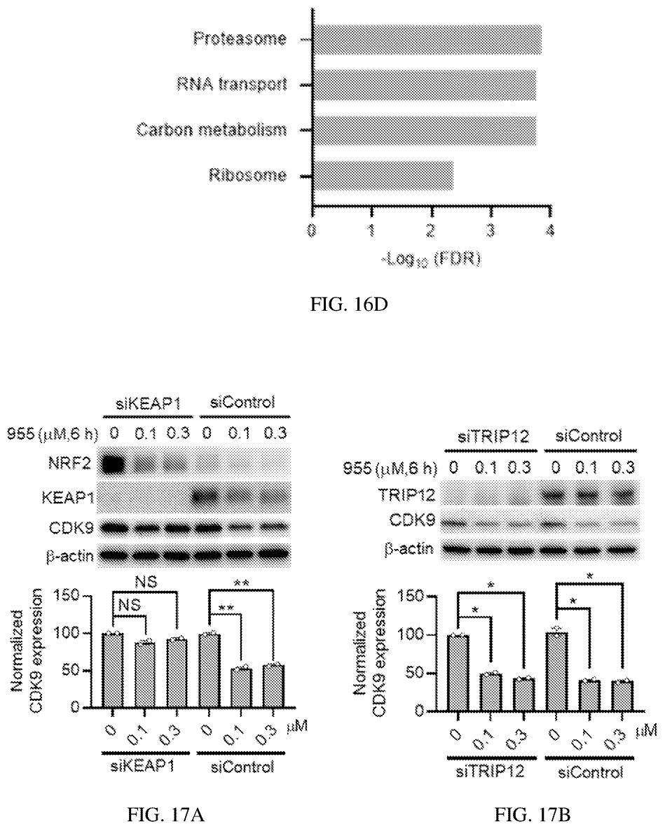

To evaluate the anti-cancer efficacy of 955, a series of experiments was conducted to compare 955 with its warhead SNS-032. The global proteome changes induced by 955 and SNS-032 in MOLT4 cells were compared. 955, but not SNS-032, reduced the protein level of CDK9 (FIGS. 5A to 5C). Interestingly, 955 also significantly decreased CDK10 (FIGS. 5A to 5D). To confirm that CDK10 is also a target protein of 955, MOLT4 cells were treated with or without proteasome inhibitor MG132 or NEDDylation inhibitor MLN4924, and the results showed that 955 also degraded CDK10 in UPS—and CRL-dependent manner (FIGS. 16A to 16D), although SNS-032 has a very low binding affinity to CDK10. This result further suggests that a weak inhibitor/binder can be converted to a potent PROTAC degrader. After 6 h treatment by either 0.1 μM 955 or 1 μM SNS-032, some shared proteins were down-regulated and those proteins were the downstream proteins regulated by CDK9, which also indicated that 955 is a more potent agent compared with the warhead SNS-032 (FIGS. 5B and 5C). With short time (1 hour) treatment, 955 only degraded CDK9 and CDK10 and downregulated one downstream protein FYTTD1 (FIG. 5A). CDK9 plays a key role in controlling basal gene transcription by interacting with RNA polymerase II. Accordingly, RNA-seq was performed to monitor the transcriptional changes in the same samples used for the proteome profiling. The results further confirmed that a lower concentration (0.1 μM) of 955 had similar effect with higher a concentration (1 μM) of SNS-032 in MOLT4 cells after 6 h treatment (FIGS. 12A to 12E). c-Myc and MCl-1 are two well-known downstream genes regulated by CDK9. Quantitative real-time PCR (qPCR) and immunoblot revealed that 955 significantly downregulated c-Myc and MCl-1 at both mRNA and protein expression levels, which were almost 10 times more effective than SNS-032 (FIGS. 12E and 12F). In addition, further analysis of RNA-level changes of the differentially expressed proteins identified by TMT-proteomics (FIG. 5A) showed that only CDK9 and CDK10 were down-regulated at the protein level and all the other proteins including FYTTD1 were mainly regulated at the RNA level (FIGS. 13A and 13B). Previous studies have shown that targeting CDK9 is an effective way to treat prostate cancer. The anti-proliferation effect of 955 was tested in various prostate cancer cell lines. Four prostate cancer cell lines, including two AR-positive cell lines (LNCap and 22RV1) and two AR-negative cell lines (PC3 and DU145), were tested. In all the four cell lines, 955 displayed anti-proliferation activities in a single digital nM range, while the EC₅₀ of SNS-032 are over 100 nM (FIG. 5G). Immunoblot results showed that 955 also degraded CDK9 and downregulated c-Myc and MCl-1 in a dose-dependent manner in LNCap cells (FIG. 5H). Collectively, these results indicate that 955 is more potent than SNS-032 to kill various liquid and solid tumor cells.

### PL-Ceritinib Conjugates Induce the Degradation of ALK-Fusion Protein

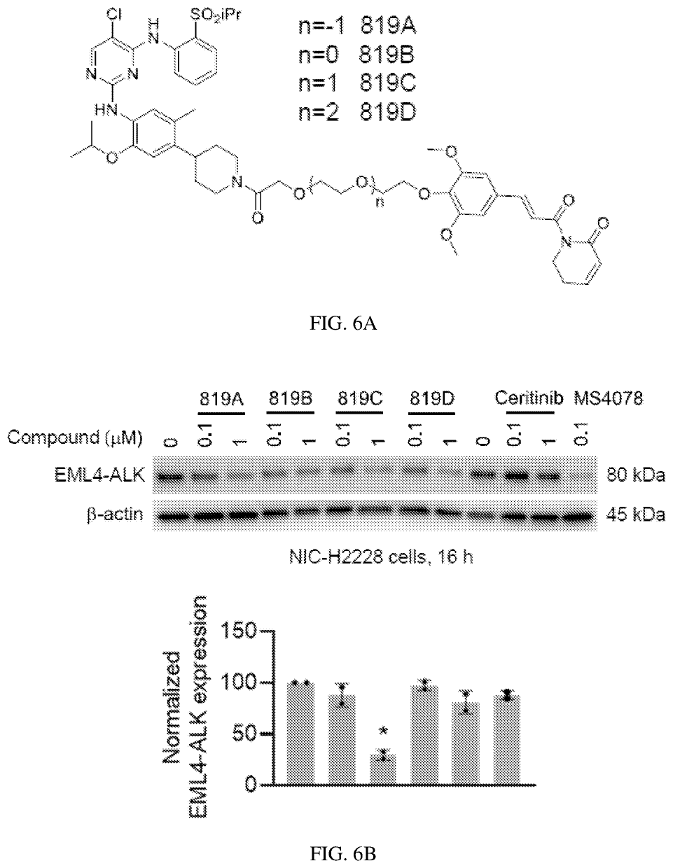

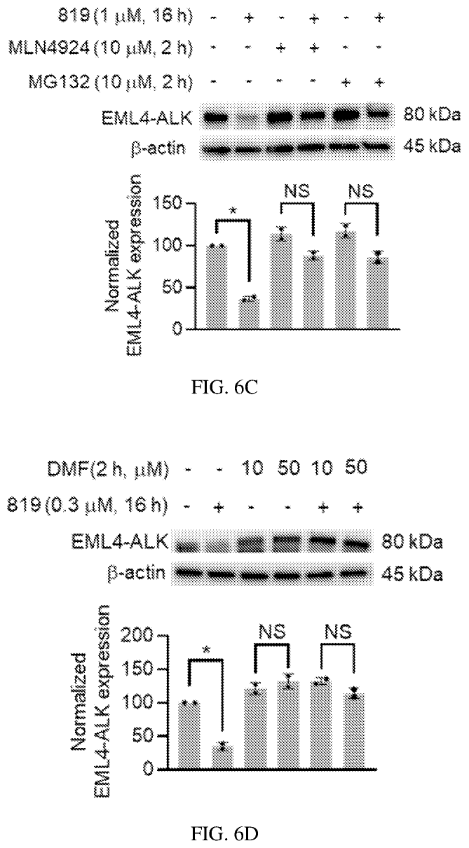

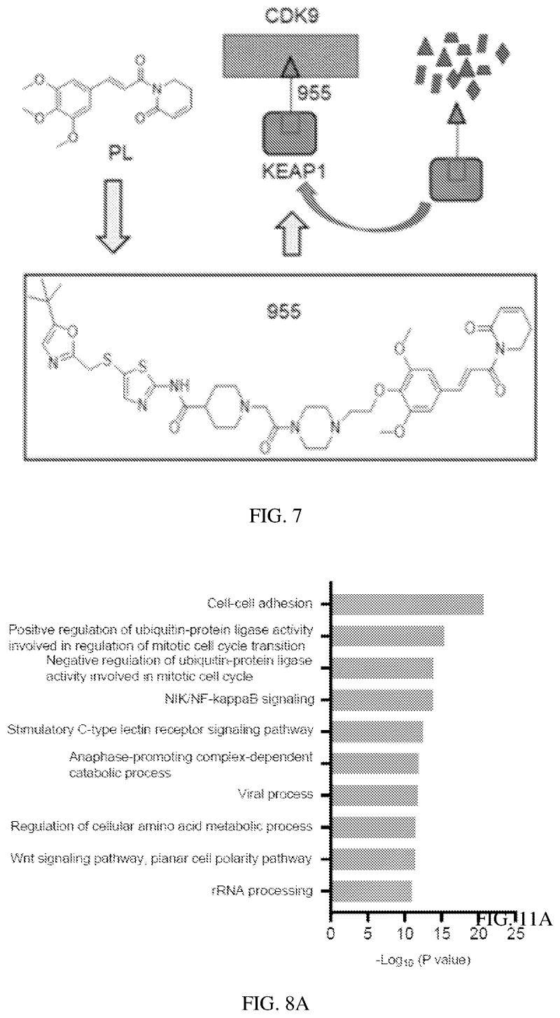

To further evaluate the potential of PL as a novel covalent E3 ligase ligand to support the degradation of other POIs, oncogenic fusion protein EML4-ALK was used as the target, as its inhibitors are suffered from drug resistance, especially in ALK-positive non-small cell lung cancer. Ceritinib, which has been used for a CRBN-ALK PROTAC design, was used as the warhead to synthesize the PL-Ceritinib conjugated compound 819 (FIG. 6A). The results showed that 819 induced the degradation of EML4-ALK in a concentration-dependent manner in NCI-H2228 cells (FIG. 6B). Similarly, this degradation effect can be blocked by MG132, MLN4924, and DMF (FIGS. 6C and 6D), demonstrating that 819A also recruits KEAP1 to mediate the degradation of EML4-ALK in NCI-H2228 cells.

Given that PL can recruit multiple E3 ligases as demonstrated by competitive ABPP assay, PL may be used as a new covalent E3 ligase ligand. For a specific POI, PL-warhead conjugates may find the best matched E3 ligase(s) to form a ternary complex to induce the degradation of this POI. 955 induced potent proteasomal degradation of CDK9. To find the E3 ligase(s) involved in CDK9 degradation, the TurboID-bait assay was used to catch the transient interacted E3 ligases and demonstrated that KEAP1 is the functional E3 ligase recruited by 955. Furthermore, PL-Ceritinib conjugated compound 819 induced degradation of ALK-fusion protein, which is the first example for covalent E3 ligase ligand-based PROTAC to degrade ALK-fusion protein.

TABLE 1
Binding affinity of the SNS-032 warhead for various CDKs

| Warhead | Binding Affinity |
|---|---|
| SNS-032 | CDK9: 4 nM |
| | CDK7: 38 nM |
| | CDK2: 62 nM |

### Example 2

PROteolysis Targeting Chimeras (PROTACs) are bifunctional molecules that degrade target proteins through recruiting E3 ligases. However, their application is limited in part because few E3 ligases can be recruited by known E3 ligase ligands. In this example, we identified piperlongumine (PL), a natural product, as a covalent E3 ligase recruiter, which induces CDK9 degradation when it is conjugated with SNS-032, a CDK9 inhibitor. The lead conjugate 955 can potently degrade CDK9 in a ubiquitin-proteasome-dependent manner and is much more potent than SNS-032 against various tumor cells in vitro. Mechanistically, we identified KEAP1 as the E3 ligase recruited by 955 to degrade CDK9 through a TurboID-based proteomics study, which was further confirmed by KEAP1 knockout and the nanoBRET ternary complex formation assay. In addition, PL-Ceritinib conjugate can degrade EML4-ALK fusion oncoprotein, suggesting that PL may have a broader application as a covalent E3 ligase ligand in targeted protein degradation.

PROteolysis TArgeting Chimeras (PROTACs) are potentially more potent anticancer therapeutics than small molecule inhibitors (SMIs) because they can degrade oncoproteins in an event-driven manner (Bondeson et al., 2015; Winter et al., 2015). Moreover, compared to SMIs that only block the catalytic function of proteins of interest (POIs), PROTACs may further remove the scaffold function of POIs by inducing their degradation. Furthermore, PROTACs may target some previously considered undruggable proteins, such as transcription factors. For example, a potent signal transducer and activator of transcription 3 (STAT3) PROTAC has been generated and shown efficacy in vivo (Bai et al., 2019). In addition, PROTAC-induced POI degradation is driven by the ternary complex formation and can be affected by the availability of lysine on the POI (Gadd et al., 2017; Khan et al., 2019; Lv et al., 2021). Therefore, PROTACs may be more specific/selective than their SMI predecessors. Because of these advantages, to date, more than 10 PROTACs have been advanced to phase I or phase II clinical trials (Mullard, 2021). The targets include androgen receptor (AR), estrogen receptor (ER), B-cell lymphoma extra-large (BCL-xL), bruton tyrosine kinase (BTK), bromodomain-containing protein 9 (BRD9), interleukin-1 receptor-associated kinase 4 (IRAK4), STAT3, and tropomyosin receptor kinase (TRK) (Mullard, 2021).

Despite great progress in the field, there are still some obstacles that prevent PROTACs from being more useful (Gao et al., 2020). Among them, to date, only a few E3 ligases and ligands are available to generate PROTACs. The human genome encodes more than 600 E3 ligases (Li et al., 2008), and only a few of them (CRBN (Winter et al., 2015), VHL (Bondeson et al., 2015), cIAPs (Naito et al., 2019), and MDM2 (Hines et al., 2019)) have been utilized by PROTACs to degrade POIs. This limits the ability to generate PROTACs for a POI that is not a suitable neo-substrate for those E3 ligases because different proteins may require different E3 ligases to effectively mediate their degradation. For example, endogenous KRAS^{G12C} can be degraded by VHL-recruiting PROTACs (Bond et al., 2020) rather than CRBN-recruiting PROTACs (Zeng et al., 2020). In addition, some E3 ligases are highly expressed in certain tumor cells (He et al., 2020). Recent studies have also shown that cancer cells develop resistance to VHL-based bromodomain and extra-terminal domain (BET) PROTACs due to loss of CUL2, as well as to CRBN-based BET and CDK9 PROTACs because of CRBN loss (Shirasaki et al., 2021; Zhang et al., 2019a). Therefore, a number of new E3 ligase ligands have been identified that can recruit AhR (Ohoka et al., 2019), DCAF11 (Zhang et al., 2021), DCAF15 (Zoppi et al., 2018), DCAF16 (Zhang et al., 2019b), FEM1B (Henning et al., 2022), KEAP1 (Tong et al., 2020; Wei et al., 2021), RNF114 (Luo et al., 2021; Spradlin et al., 2019), and RNF4 (Ward et al., 2019) E3 ligases to degrade POIs. Based on mathematical modeling (Chaudhry, 2021) and previous studies (Gabizon and London, 2021; Kiely-Collins et al., 2021; Spradlin et al., 2019; Wei et al., 2021; Zhang et al., 2019b), covalent E3 ligase ligand-based PROTACs may outperform non-covalent E3 ligase ligand-based PROTACs due to better kinetics of ternary complex formation, and minimal perturbation of its endogenous substrates with low fractional occupancy of the recruited E3 ligase. In addition, a covalent E3 ligase ligand in a PROTAC may provide additional selectivity for a given POI (Spradlin et al., 2019; Ward et al., 2019; Zhang et al., 2019b). Identifying more E3 ligase ligands may further expand the toolbox, overcome the drug resistance, and potentially generate more potent and specific PROTACs.

Piperlongumine (PL, FIG. 1A) is a natural product that exhibits potent antitumor activity (Roh et al., 2014), in part via induction of oxidative stress through its two Michael acceptors that can covalently react with GSTP1 (Harshbarger et al., 2017) and GSTO1 (Li et al., 2019). Our previous studies also showed that PL can selectively kill senescent cells in part through induction of OXR1 degradation in a proteasome-dependent manner (Wang et al., 2016; Zhang et al., 2018b). In addition, we found that PL can bind several intracellular proteins in senescent cells, including 8 different E3 ligases (Zhang et al., 2018b). SNS-032 is a CDK inhibitor with antitumor activity. It can potently inhibit CDK9 (IC₅₀=4 nM), CDK2 (IC₅₀=38 nM), and CDK7 (IC₅₀=62 nM), and also has moderate inhibitory activity for a panel of other kinases (Chen et al., 2009). Interestingly, when it was conjugated with thalidomide, the conjugate (THAL-SNS-032) became more specific and could only degrade CDK9 but not other known SNS-032 targets (Olson et al., 2018). To test if PL can be used as a new covalent E3 ligase ligand, we linked PL to SNS-032, and found that the PL-SNS-032 conjugates potently induced CDK9 degradation. Mechanistically, we demonstrated that the lead PL-SNS-032 conjugate, 955, degraded CDK9 in a ubiquitin-proteasome system (UPS) dependent manner. Using the TurboID-based proteomics combined with siRNA knockdown, CRISPR-Cas9 gene knockout, and the nanoBRET ternary complex formation assay, we identified and confirmed KEAP1 as the E3 ligase recruited by 955 to degrade CDK9. Furthermore, a PL-based ALK PROTAC can potently degrade ALK-fusion protein in ALK positive non-small cell lung cancer (NSCLC) cells. Our results demonstrate that PL has the potential to be used as a new covalent E3 ligase ligand to generate PROTACs to degrade different target proteins through recruiting KEAP1. In addition, our study reveals that the TurboID-based proteomics is a very useful technology to identify E3 ligases recruited by natural products for targeted protein degradation.

### Generation of PL-Conjugated CDK9 PROTACs

Our previous study showed that PL can pull down 8 E3 ligases in senescent cells (Zhang et al., 2018b). Here, we used a competitive activity-based protein profiling (ABPP) assay with the PL-Alkyne as a probe to validate whether PL can covalently bind E3 ligases in cancer cells as well (FIG. 1A). The assay was done using MOLT4 human T-cell acute lymphoblastic leukemia (T-ALL) cells as illustrated in FIG. 1B. To exclude proteins that bound non-specifically to PL-Alkyne, we also included a sample in which the cells were pre-treated with a high concentration of PL before the addition of PL-Alkyne to compete for protein binding (FIG. 1B). The western blot result showed that PL-Alkyne pulled down many proteins, which could be effectively competed by the pre-treatment with PL (FIG. 1C), suggesting that most of the pulled-down proteins by the probe are PL-binding proteins. The MS results showed that PL can bind to about 300 proteins (FIG. 1D and Table 2), including GSTO1, a previously identified PL target (Li et al., 2019). We performed gene ontology (GO) and Kyoto encyclopedia of genes and genomes (KEGG) pathway enrichment analyses of those PL-binding proteins and found that many of the proteins involved in the UPS were highly enriched (FIGS. 16A to 16D and Table 2), including 9 E3 ligases (FIG. 1D). These findings indicate that PL has the potential to be used as a new E3 ligase ligand for PROTAC design.

CDK9 is a well-established cancer target and can be effectively degraded by a PROTAC (THAL-SNS-032) consisting of thalidomide, a linker, and the CDK inhibitor SNS-032 (Olson et al., 2018). To test our hypothesis, we generated a series of PL-SNS-032 bifunctional molecules with linkers of different types and lengths (FIGS. 9A, 9B) and examined their potencies in degrading CDK9 in MOLT4 cells (FIGS. 9A to 9C). These preliminary assays identified 955 for further evaluation and characterization because of its high potency in these assays (FIG. 2A). Especially, 955 potently degraded CDK9 with a DC₅₀ value of 9 nM after 16 h treatment in MOLT4 cells (FIGS. 9B, 9C). Even with short-term treatment (6 h), 955 was able to potently degrade CDK9 while the warhead SNS-032 could not (FIG. 2B). In addition, 955 was more potent in inducing PARP cleavage than SNS-032 (FIG. 2B). The time-course study showed that 955 induced almost complete degradation of CDK9 at 0.1 μM and within 8 h of treatment (FIG. 2C) and its effect lasted up to 18 h after the removal of 955 from the culture (FIG. 2D). Similar results were also observed in 293T cells (FIG. 10C) and K562 cells (FIG. 10D). To exclude the possibility that the degradation of CDK9 is caused by the combination effect of PL and SNS-032, we treated MOLT4 cells with either PL or SNS-032 alone or their combination and found that none of these treatments degraded CDK9 (FIG. 2E). However, pre-treatment of MOLT4 cells with PL or SNS-032 blocked 955-induced CDK9 degradation (FIG. 2F).

### Mechanism of Action of 955 in Degrading CDK9

To confirm that 955 functions as a PROTAC to degrade CDK9, we performed a series of mechanistic studies using inhibitors to block different protein degradation pathways. First, we treated MOLT4 cells with vehicle, the proteasome inhibitor MG132, or bortezomib prior to 955 treatment. The results showed that the degradation of CDK9 can be blocked by the two different proteasome inhibitors (FIG. 3A). In contrast, pre-treatment with either of the two lysosome inhibitors, Baf-A1 and chloroquine, or with the pan-caspase inhibitor QVD does not block CDK9 degradation by 955 (FIGS. 3B, 3C). These results demonstrate that 955 induces CDK9 degradation through the proteasome but not lysosome and activated caspases. Furthermore, we used two E1 inhibitors, PYR-41 and TAK-243, to verify that 955-induced CDK9 degradation is E1-dependent (FIGS. 3D, 3E). In our competitive ABPP assay, 9 PL-recruited E3 ligases were identified by the LC-MS/MS (FIG. 1D), and thus we next investigated which type of E3 ligase(s) is involved in the degradation of CDK9 by 955. MLN4924 is a NEDD8-activating enzyme (NAE) inhibitor and can selectively inhibit cullin RING-related ubiquitin E3 ligase(s) (CRLs) through blocking the neddylation of cullin (Kawakami et al., 2001; Sakata et al., 2007). We found that MLN4924 pre-treatment blocks 955-induced CDK9 degradation (FIG. 3E), suggesting that a CRL is likely to be recruited by 955 to degrade CDK9. Similar results were also observed in 293T and K562 cells (FIGS. 11A to 11F). Collectively, these findings suggest that 955 degrades CDK9 in a UPS-dependent manner probably via recruiting a CRL. Furthermore, we investigated whether 955 degrading CDK9 relies on PL to covalently recruit an E3 ligase via its two Michael acceptors by generating 336, in which the C2-C3 and C7-C8 double bonds of PL are reduced (FIG. 3F). We found that 955, but not 336, can induce CDK9 degradation (FIG. 3H), indicating that 955 degrades CDK9 via covalently recruiting a CRL through PL's Michael acceptors.

### Identification of the E3 Ligase(s) Recruited by 955 to Degrade CDK9

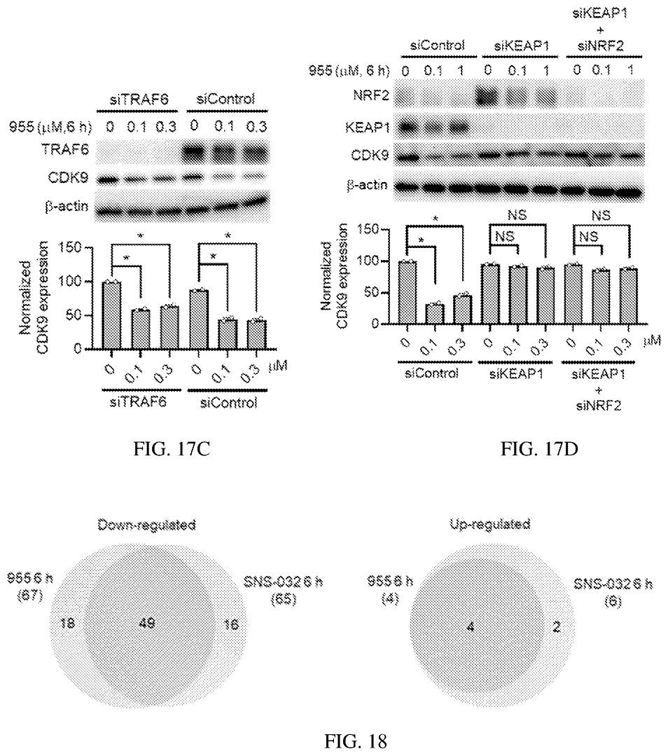

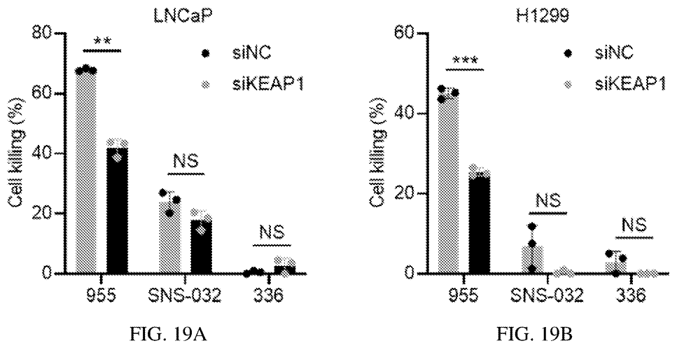

PROTACs induce the degradation of target proteins by hijacking an E3 ligase. However, it is usually difficult to directly identify the E3 ligase recruited by a new E3 ligase ligand in a PROTAC using the co-immunoprecipitation method because the formation of the complex is transient and very dynamic (Liu et al., 2020). TurboID assay is a powerful proximity labeling method that can sensitively detect weak and transient protein-protein interactions by biotinylating proteins that interact with a bait protein fused with an engineered biotin ligase (Branon et al., 2018). Similar biotin-based proximity labeling assays, such as APEX2 assay (Mayor-Ruiz et al., 2019) and AirID assay (Kido et al., 2020), have been used to study the degrader-induced or inhibitor-blocked E3 ligase: substrate interactions in cells. Therefore, we adapted TurboID technology to identify and characterize the specific E3 ligase(s) recruited by 955 to mediate CDK9 degradation by ectopically expressing V5-TurboID-CDK9 in 293T cells and comparing the biotinylated proteins with or without 955 treatment (FIG. 4A). The results from this assay showed that 955 can induce the interaction of CDK9 with 6 different E3 ligases (FIG. 4B, Table 3). To compare the PL-binding E3 ligases identified by the competitive ABPP assay (FIG. 2D) with those identified by the TurboID-bait assay (FIG. 4B), we found that KEAP1 is the only CRL identified by both methods. A previous study showed that KEAP1 can be recruited to degrade Tau protein by a KEAP1-based peptide PROTAC (Lu et al., 2018). In addition, two recent studies discovered KEAP1 E3 ligase ligands that can be used to recruit KEAP1 to degrade BRD4 after linking to the BRD4 inhibitor JQ1 (Tong et al., 2020; Wei et al., 2021). To determine whether KEAP1 is the E3 ligase recruited by 955 to degrade CDK9, we first used immunoblot to confirm the results of the TurboID-bait assay. We found that significantly more KEAP1 can be pulled down after 955 treatment than without 955 treatment in the presence of biotin. By contrast, R-actin cannot be pulled down with or without 955 treatment under the same experimental conditions (FIG. 4C). To further validate that KEAP1 is recruited by 955 to mediate CDK9 degradation, we treated MOLT4 cells with the known KEAP1 covalent inhibitors CDDO-ME, CDDO-IM, and dimethyl fumarate (DMF) (Suzuki and Yamamoto, 2017), prior to the addition of 955. Treatment with these inhibitors completely blocked 955-induced CDK9 degradation (FIGS. 4D, 4E). Furthermore, we used both siRNA and CRISPR-Cas9 to knock down/out of KEAP1 and found that CDK9 degradation induced by 955 was blocked after depleting KEAP1 (FIGS. 4F and 20A). We also knocked down two additional E3 ligases (TRIP12 and TRAF6) identified by TurboID-bait assay using siRNAs, which had no effect on 955-induced CDK9 degradation (FIGS. 17B, 17C). Ectopic expression of Halo-tagged KEAP1 in the KEAP1 stable knockout cells confirmed that the expression of KEAP1 rescued CDK9 degradation induced by 955 (FIG. 4G), suggesting KEAP1 is likely the primary E3 ligase recruited by 955 to degrade CDK9. Depleting KEAP1 can stabilize NRF2 (FIG. 17A). To rule out the possibility that the upregulation of NRF2 and its downstream pathways are involved in metabolizing 955 to prevent it from being an effective PROTAC degrader, we simultaneously knocked down both KEAP1 and NRF2 in cells. The results from this study show that knocking down NRf2 does not reverse the effect of KEAP1 knocking down on abrogation of 955-induced CDK9 degradation, confirming that 955 induces CDK9 degradation by recruiting KEAP1 but not by activation of NRF2 (FIG. 17D). To further confirm that 955 recruits KEAP1 to degrade CDK9 through covalent binding, we performed a gel-based competitive ABPP assay, in which we used serially diluted 955 to compete with iodoacetamide (IA)-alkyne, a non-selective cysteine-reactive probe, for binding to purified KEAP1 protein. The result from this assay confirms that 955 can compete with IA-alkyne to covalently bind to KEAP1 in a concentration-dependent manner (FIG. 4H). Lastly, forming a POI: PROTAC: E3 ternary complex may be a necessary step for the induction of POI ubiquitination and degradation. Therefore, we fused CDK9 with HiBit-tag and KEAP1 with Halo-tag to perform nanoBRET assay to further test if 955 can induce the formation of the CDK9: 955: KEAP1 ternary complex in live cells (FIG. 4I). The results demonstrated that 955 can induce the formation of the CDK9: 955: KEAP1 ternary complex in a dose-dependent manner while the induction is negligible by 336, suggesting that the formation of this ternary complex may be dependent on the covalent binding of 955 to KEAP1 via the PL Michael acceptor(s) (FIG. 4I). In conclusion, 955 can covalently hijack KEAP1 to mediate CDK9 degradation.

955 is a More Potent Anti-Cancer Agent than SNS-032

To evaluate the anti-cancer efficacy and specificity of 955, a series of experiments were conducted to compare 955 with its warhead SNS-032. We first compared the global proteome changes induced by 955 and SNS-032 in MOLT4 cells. As expected, cells exhibited a significant reduction of CDK9 after treatment with 0.1 μM 955 for 1 h and 6 h while treatment with 1 μM SNS-032 for 6 h had no significant effect on CDK9 (FIGS. 5A to 5C and Table 4). Interestingly, 955, but not SNS-032, can also potently degrade CDK10 (FIGS. 5A to 5D, and Table 4) in a proteasome- and CRL-dependent manner because pre-treatment with MG132 and MLN4924 can block the degradation (FIG. 5E). The previously reported CRBN-based CDK9 PROTAC (THAL-SNS-032) also has a similar but weaker effect on inducing CDK10 degradation (Olson et al., 2018), which can be explained by the moderate binding between SNS-032 and CDK10 confirmed by Kinativ screening in MOLT4 lysates (Olson et al., 2018). Because CDK9 plays an important role in regulation of gene transcription, inhibition/degradation of CDK9 may lead to reduction of many short half-life proteins including c-Myc and MCL-1 (Rahaman et al., 2016). These downstream proteins may further regulate the expression of their downstream targets. Therefore, it is not uncommon to see that more proteins are downregulated in cells after longer treatment with a CDK9 inhibitor/degrader than shorter treatment. This may explain why more proteins were downregulated in cells after 6 h treatment with 955 than in cells after 1 h treatment. Importantly, 955 reduced the expression of CDK9 and CDK10 at both time points whereas SNS-032 had no effect on their expression, suggesting that CDK9 and CDK10 are likely the primary and direct targets of 955. This suggestion is further supported by the findings that most of other proteins were similarly downregulated in cells 6 h after 955 and SNS-032 treatment (Blue dots shown in FIGS. 5A to 5C and 21). Collectively, these findings suggest that 955 can also downregulate CDK9 downstream targets as SNS-032 but via a different mechanism, i.e. via degradation of CDK9.

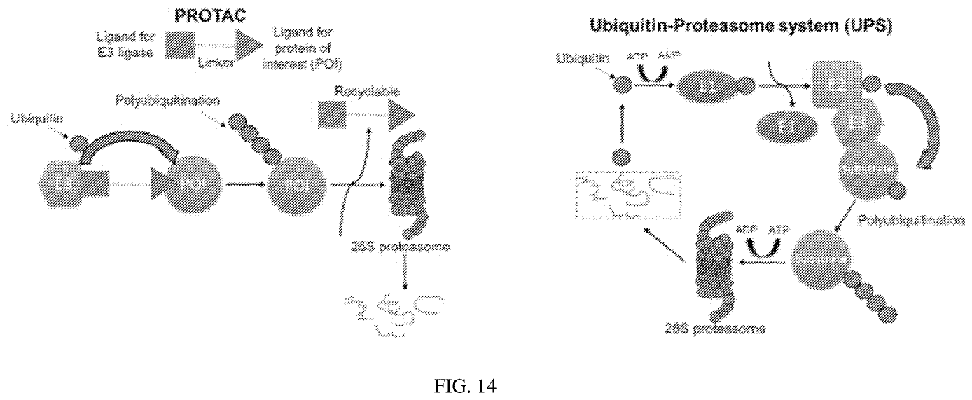

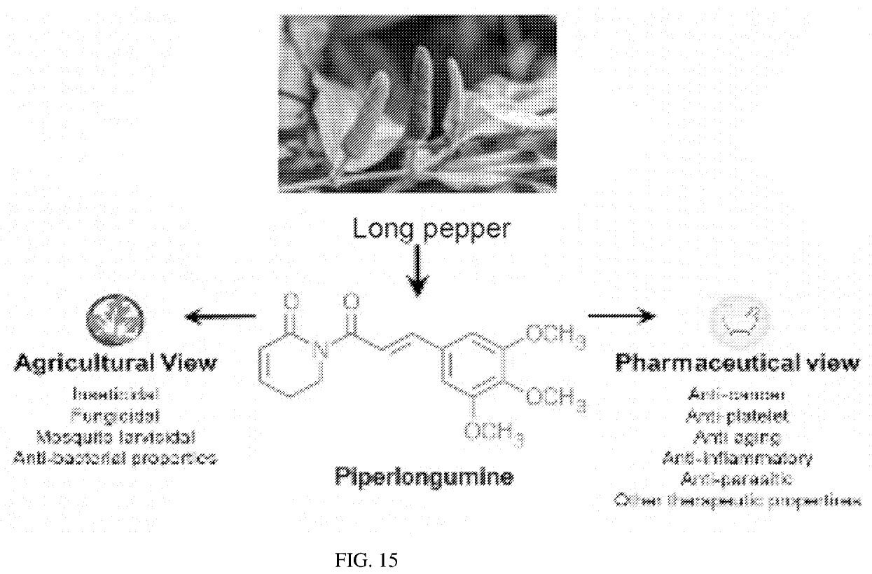

CDK9 plays a key role in regulating gene transcription by interacting with RNA polymerase II (Rahaman et al., 2016). Therefore, we further performed RNA-seq to analyze the transcriptional changes in the same samples used for the proteome profiling. The results from this analysis confirmed that 955 is more potent than SNS-032 in the down-regulation of CDK9-dependent transcription in MOLT4 cells after 6 h treatment (FIGS. 12A, 12B, 14A, 14B, and Table 5). In addition, MOLT4 cells treated with 0.1 μM 955 and 1 μM SNS-032 displayed very similar proteomic and transcriptomic profiles, suggesting that 955 is more potent than SNS-032 in downregulating CDK9 downstream targets and its effects are not caused by PL. This suggestion is further supported by the analyses of c-Myc and MCl-1, two well-known downstream targets of CDK9 (Rahaman et al., 2016). Specifically, quantitative real-time PCR (qPCR) and immunoblot revealed that 955 is about 10-fold more potent than SNS-032 in downregulation of c-Myc and MCl-1 expression at the levels of both mRNA and protein (FIGS. 12E, 12F). We further compared the antiproliferative effect of 955 with SNS-032 in prostate cancer cell lines since previous studies showed that targeting CDK9 is an effective way to inhibit prostate cancer (Rahaman et al., 2016). Two AR-positive (LNCaP and 22RV1) and two AR-negative (PC3 and DU145) cell lines were studied. We found that 955 exhibits antitumor activities against all four cell lines with EC₅₀ values in the single digital nM range, while the EC₅₀ values of SNS-032 against these cell lines are over 100 nM (FIG. 5G). In addition, PL itself had no anti-proliferative effect within 3 M and combined treatment of PL and SNS-032 showed a similar EC₅₀ to SNS-032 in all the tested prostate cancer cell lines, which further confirmed that 955 is more potent than SNS-032 against the tumor cells (FIG. 5G). Furthermore, immunoblot assays showed that 955 potently degraded CDK9 and downregulated c-Myc and MCL-1 in LNCaP cells (FIG. 5H). Collectively, these results indicate that 955 is more potent than SNS-032 against tumor cells. To further test if the E3 ligase KEAP1 recruited by 955 is relevant to its antiproliferative activity, we knocked down KEAP1 in LNCaP prostate cancer cells and H1299 lung cancer cells and measured the cell-killing effects in KEAP1 siRNA- and control siRNA-treated cells. The results confirmed that knockdown of KEAP1 attenuated the cytotoxicity of 955, but had no significant effect of the warhead SNS-032 and compound 336 that does not recruit KEAP1 (FIGS. 41, 22A, to 22B). These results provide additional support that 955 kills tumor cells primarily via degrading CDK9 by recruiting the E3 ligase KEAP1.

### The PL-Ceritinib Conjugate Degrades ALK-Fusion Oncoprotein

To further evaluate the potential of PL as a novel covalent KEAP1 ligand to generate PROTACs for the degradation of oncoproteins, we synthesized several PL-ceritinib conjugates to test if these compounds can degrade EML4-ALK, an oncogenic fusion protein in NSCLC (FIG. 6A) because EML4-ALK-positive NSCLC patients frequently develop resistance to the anaplastic lymphoma kinase (ALK) inhibitor ceritinib (Pan et al., 2021). In addition, ceritinib has been used for generating a CRBN-ALK PROTAC (Zhang et al., 2018a). Here we showed one of those conjugated compounds, 819, can degrade EML4-ALK in a concentration-dependent manner in NCI-H2228 NSCLC cells (FIG. 6B). The degradation of EML4-ALK can be blocked by the pre-treatment of the cells with MG132, MLN4924, and DMF (FIGS. 6C, 6D), suggesting that 819 may also recruit KEAP1 to mediate the degradation of EML4-ALK in NCI-H2228 cells in a UPS-dependent manner. With further optimization of the linker, we expect that PL-ceritinib conjugates can more potently degrade EML4-ALK in NSCLC cells.

### CONCLUSION

Despite the human proteome encoding more than 600 E3 ligases, only a few E3 ligases have been used for PROTAC design due to the limited availability of E3 ligase ligands. Therefore, finding new E3 ligase ligands to expand the toolbox of PROTAC technology may be important for the further development of this field. Here, we design and synthesize a series of PL-SNS-032 conjugates and find that 955 potently induces CDK9 degradation in a UPS-dependent manner. To find the E3 ligase(s) recruited by 955, we use the TurboID-bait assay to identify the proteins that transiently interact with CDK9 induced by 955. We found that KEAP1 is the CDK9 degradation E3 ligase recruited by 955 through covalent binding of PL, which is further confirmed genetically by siRNA and CRISPR-Cas9 knock down/out and rescue assays. Furthermore, EML4-ALK protein can also be successfully degraded by the PL-ceritinib conjugate 819, which provides additional evidence to demonstrate that PL can be used as a KEAP1 recruiter to induce the degradation of different POIs. Compared with the other two commonly used E3 ligases (VHL and CRBN), KEAP1 is highly expressed in many tumor cells such as lung, kidney, breast, prostate, and brain cancer cells. Therefore, PL-based PROTACs may achieve better degradation efficacy in those cancer cells. Additionally, with a relatively smaller molecular size (MW=317.3 Da) of PL, PL-based PROTACs might possess more favorable physicochemical properties for drug development after further characterization of the binding mode of PL with KEAP1 and optimization to improve its specificity. Taken together, our study demonstrates that natural products are an important source for the discovery of new E3 ligase ligands and TurboID-bait assay is a powerful tool to identify E3 ligases recruited by nature compounds and novel E3 ligase ligands.

Key Resources Table

| | | |
|---|---|---|
| REAGENT or RESOURCE | SOURCE | IDENTIFIER |

Antibodies

| | | |
|---|---|---|
| Rabbit monoclonal anti-Biotin (HRP | Cell Signaling | Cat. No. 5571S; RRID: |
| Conjugate) | Technology | AB_10892862 |
| Rabbit monoclonal anti-PARP | Cell Signaling | Cat. No. 9542S; RRID: |
| | Technology | AB_2160739 |
| Rabbit monoclonal anti-c-Myc | Cell Signaling | Cat. No. 5605S; RRID: |
| | Technology | AB_1903938 |
| Rabbit monoclonal anti-MCL1 | Cell Signaling | Cat. No. 5453S; RRID: |
| | Technology | AB_10694494 |
| Mouse monoclonal anti-ALK | Cell Signaling | Cat. No. 3791S; RRID: |
| | Technology | AB_1950402 |
| Rabbit monoclonal anti-KEAP1 | Proteintech | Cat. No. 10503-2-AP; RRID: |
| | | AB_2132625 |
| Mouse monoclonal anti-CDK9 | Santa Cruz | Cat. No. sc-13130; RRID: |
| | | AB_627245 |
| Rabbit monoclonal anti-V5-tag (HRP | Bethyl | Cat. No. A190-120P; RRID: |
| Conjugate) | | AB_162729 |
| Rabbit anti-TRIP12 Antibody | Fortis | Cat. No. A301-814A-M |
| TRAF6 (D21G3) Rabbit | Cell Signaling | Cat. No. 8082 |
| | Technology | |
| NRF2, NFE2L2 Polyclonal antibody | Proteintech | Cat. No. 16396-1-AP |
| Mouse monoclonal anti-actin | MP Biomedicals | Cat. No. 8691001; RRID: |
| | | AB_2920628 |

Recombinant DNA

| | | |
|---|---|---|
| HA-CDK9 | Addgene | RRID: Addgene_28102 |
| pet28a-His6-Keap1 | Addgene | RRID: Addgene_62454 |
| pMIR-DsRed-IRES-His-Halo-Tev- | | RRID: Addgene_58240 |
| Keap1 | Addgene | |
| V5-TurboID-NES_pCDNA3 | Addgene | RRID: Addgene_107169 |
| V5-TurboID-CDK9 | | N/A |
| HiBit-CDK9 | | N/A |

sgRNA sequence for KEAP1 knockout

| | | |
|---|---|---|
| sg1: GTACGCCTCCACTGAGTGCA | Abm | Cat. No. 254911110595 |
| sg2: TGACAGCACCGTTCATGACG | Abm | Cat. No. 254911110595 |

siRNA

| | | |
|---|---|---|
| KEAP1 siRNA (h) | Santa Cruz | Cat. No. sc-43878 |
| Control siRNA-A | Santa Cruz | Cat. No. sc-37007 |
| TRIP12 siRNA (h) | Santa Cruz | Cat. No. sc-94390 |
| TRAF6 siRNA (h) | Santa Cruz | Cat. No. sc-36717 |
| NRF2 siRNA (h) | Santa Cruz | Cat. No. sc-37030 |
| MYC primers: forward 5′- | | N/A |
| GGCTCCTGGCAAAAGGTCA-3′ | | |
| and | | |
| reverse | | |
| 5′-CTGCGTAGTTGTGCTGATGT-3′ | | |

Primers for the construction of the
pcDNA3-V5-TurboID-CDK9 plasmid

| | |
|---|---|
| CDK9 fragment cloning: 5′- | N/A |
| TGGCTCGAGCGGTGGGAATTCTG | |
| GTatggcaaagcagtacgactcggtggag-3′ | |
| and | |
| 5′-TCTTCCGCTAGCTCCACCGGATCC | |
| TCCTCAgaagacgcgctcaaactccgtctggt-3′ | |
| V5-TurboID fragment clloning pair 1: | N/A |
| 5′-ACCAGACGGAGTTTGAGCGCGTC | |
| TTCTGActgcctcccctggagcgcctgaccctg-3′ | |
| and | |
| 5′-caatgatgagcacttttaaagtt | |
| ctgctatgtggcgcggtattatcccgtattgac-3′ | |
| V5-TurboID fragment clloning pair 2: | N/A |
| 5′-gtcaatacgggataataccgcgccacatag | |
| cagaactttaaaagtgctcatcattg-3′ | |
| and | |
| 5′-ACTCCACCGAGTCGTACTGCTTTG | |
| CCATctgcagcttttcggcagaccgcagactg-3′ | |

Primers for the construction of the HiBit-CDK9 plasmid

| | |
|---|---|
| vector-containing fragment cloning: | N/A |
| 5′-accagacggagtttgagcgcgtcttcTGAGG | |
| AGGATCCGGTGGAGCTAGCGGAAGA-3′ | |
| and | |
| 5′-ctccaccgagtcgtactgcttcgccatACCA | |
| GAATTCCCACCGCTCGAGCCA-3′ | |
| CDK9 fragment cloning: 5′- | N/A |
| TGGCTCGAGCGGTGGGAATTCTG | |
| GTatggcaaagcagtacgactcggtggag-3′ | |
| and | |
| 5′-TCTTCCGCTAGCTCCACCGGATCC | |
| TCCTCAgaagacgcgctcaaactccgtctggt-3′ | |
| Recombinant Proteins | |
| His-KEAP1 | N/A |

Experimental Models: Cell Lines

| | | |
|---|---|---|
| Human: T-ALL MOLT4 | ATCC | Cat. No. CRL-1582 |
| Human: K562 | ATCC | Cat. No. CCL-243 |
| Human: NCI-H1299 | ATCC | Cat. No. CRL-5803 |
| Human: NCI-H2228 | ATCC | Cat. No. CRL-5935 |
| Human: LNCaP | ATCC | Cat. No. CRL-1740 |
| Human: 22RV1 | ATCC | Cat. No. CRL-2505 |
| Human: PC3 | ATCC | Cat. No. CRL-1435 |
| Human: DU145 | ATCC | Cat. No. HTB-81 |
| Human: HEK 293T | ATCC | Cat. No. ACS-4500 |

Critical Commercial Assays

| | | |
|---|---|---|
| NanoBRET assay | Promega | Cat. No. CS1956B03 |

Deposited Data

| | | |
|---|---|---|
| RNA sequencing data | Gene Expression | GSE206612 |
| | Omnibus (GEO) | |

Software and Algorithms

| | | |
|---|---|---|
| GraphPad Prism | Graphpad | www.graphpad.com/ |
| | Software Inc | scientific-software/prism/ |
| MaxQuant | Max Planck | maxquant.org/ |
| | Institute | |
| Scaffold Q + S | Proteome | www.proteomesoftware.com/ |
| | Software | products/scaffold-qs |
| Galaxy | Web-based | usegalaxy.org/ |
| | platform | |
| ImageJ | NIH | imagej.nih.gov/ij/ |

### Biology Materials and Methods

### Cell Lines

Human T-ALL MOLT4 (Cat. No. CRL-1582), K562 (Cat. No. CCL-243), NCI-H1299 (Cat. No. CRL-5803), NCI-H2228 (Cat. No. CRL-5935), LNCaP (Cat. No. CRL-1740), 22RV1 (Cat. No. CRL-2505), PC3 (Cat. No. CRL-1435), DU145 (Cat. No. HTB-81), and epithelial kidney HEK 293T (293T, Cat. No. ACS-4500) cell lines were recently purchased from American Type Culture Collection (ATCC, Manassas, VA, USA). MOLT4, K562, NCI—H-1299, NCI-H2228, LNCap, and 22RV 1 cell lines were cultured in RPMI 1640 medium (Cat No. 22400-089, Thermo Fisher Scientific, Waltham, MA, USA) supplemented with 10% (v/v) heat-inactivated fetal bovine serum (FBS, Cat. No. S11150H, Atlanta Biologicals, Flowery Branch, GA, USA), 100 U/mL penicillin and 100 pig/mL streptomycin (Pen-Strep, Cat. No. 15140122, Thermo Fisher Scientific). PC3 cells were cultured in complete F—12K medium (Kaighn's modification of Ham's F-12 medium, Cat. No. 30-2004, ATCC) supplemented with 10% (v/v) heat-inactivated FBS, 100 U/mL penicillin, and 100 μg/mL streptomycin. DU145 cells were cultured in complete Eagle's Minimum Essential Medium (EMEM, Cat. No. 30-2003, ATCC) supplemented with 10% (v/v) heat-inactivated FBS, 100 U/mL penicillin, and 100 μg/mL streptomycin. 293T cells were cultured in complete Dulbecco's modified Eagle medium (DMEM, Cat. No. 12430054, Thermo Fisher Scientific) with 10% (v/v) heat-inactivated FBS, 100 U/mL penicillin, and 100 μg/mL streptomycin. All the cell lines were maintained in a humidified incubator at 37° C. and 5% CO₂.

### Immunoblotting

Cells were collected and lysed in RIPA lysis buffer (Cat. #BP-115DG, Boston Bio Products, Ashland, MA, USA) supplemented with protease and phosphatase inhibitor cocktails (Cat. #PPC1010, Sigma-Aldrich, St. Louis, MO, USA). Protein samples were made and immunoblotting was performed as described previously (Khan et al., 2019; Lv et al., 2021). Antibodies purchased from Cell Signaling Technologies (CST) and the dilutions are as follows: Biotin (Cat. No. 5571S, 1:1000), PARP (Cat. No. 9542S, 1:1000), c-Myc (Cat. No. 5605S, 1:1000), MCL1 (Cat. No. 5453S, 1:1000), and ALK (Cat. No. 3791S, 1:1000). KEAP1 antibody was purchased from Proteintech (Cat. No. 10503-2-AP, 1:1000). CDK9 antibody was purchased from Santa Cruz (Cat. No. sc-13130, 1:500). V5-tag antibody was purchased from Bethyl (Cat. No. A190-120P, 1:1000). j-actin antibody was purchased from MP Biomedicals (Cat. No. 8691001, 1:20 000).

### Viability Assay

Viability (MTS) assays in various cancer cells followed the methods described in our previous report (Khan et al., 2019). The data were expressed as average % cell viability and fitted in non-linear regression curves using GraphPad Prism 9 (GraphPad Software, La Jolla, CA, USA).

### Competitive ABPP and LC-MS/MS

Competitive ABPP. MOLT4 cells (2×10⁷) were pre-treated with DMSO or 20 M PL for 4 h and then treated with 1 μM PL-Alkyne probe (Zhang et al., 2018b) for an additional 4 h. The cells were then harvested and lysed in 1× Dulbecco's phosphate-buffered saline (PBS) buffer (Cat. #D1408, Sigma-Aldrich) containing protease and phosphatase inhibitors. CuAAC reaction was performed as previously described (Spradlin et al., 2019) and biotinylated proteins were enriched using Pierce™ streptavidin agarose beads (Cat. #20353, ThermoFisher).

Sample preparation and LC-MS/MS analysis. Protein samples were reduced, alkylated, and digested on-bead using filter-aided sample preparation (Wisniewski et al., 2009) with sequencing grade modified porcine trypsin (Cat. No. V5111, Promega). Nano-liquid chromatography tandem mass spectrometry (Nano-LC/MS/MS) was performed on a Thermo Scientific Q Exactive HF Orbitrap mass spectrometer equipped with an EASY Spray nanospray source (Thermo Scientific) operated in positive ion mode. The LC system was an UltiMate™ 3000 RSLCnano system (Thermo Scientific). The mobile phase A was water containing 0.1% formic acid and the mobile phase B was acetonitrile with 0.1% formic acid. The mobile phase A for the loading pump was water containing 0.1% trifluoracetic acid. 5 L of sample is injected onto a PharmaFluidics pPAC^{T}M C18 trapping column (C18, 5 m pillar diameter, 10 mm length, 2.5 m inter-pillar distance). at 10 L/ml flow rate. This was held for 3 min and washed with 1% B to desalt and concentrate the peptides. The injector port was switched to inject, and the peptides were eluted off of the trap onto the column. PharmaFluidics 50 cm pPAC™ was used for chromatographic separations (C18, 5 m pillar diameter, 50 cm length, 2.5 m inter-pillar distance). The column temperature was maintained 40° C. A flowrate of 750 nl/min was used for the first 15 min and then the flow was reduced to 300 nl/min. Peptides were eluted directly off the column into the Q Exactive system using a gradient of 1% B to 20% B over 100 minutes and then to 45% B in 20 minutes for a total run time of 150 minutes:

| Time (min) | % B | Flow Rate (nL/min) |
|---|---|---|
| 0 | 1 | 750 |
| 3 | 1 | 750 |
| 15 | 5 | 750 |
| 15.1 | 5 | 300 |
| 100 | 20 | 300 |
| 123 | 45 | 300 |
| 130 | 95 | 300 |
| 135 | 95 | 300 |
| 135.1 | 1 | 300 |
| 150 | 1 | 300 |

The MS/MS was acquired according to standard conditions established in the lab. The EASY Spray source operated with a spray voltage of 1.5 KV and a capillary temperature of 200° C. The scan sequence of the mass spectrometer was based on the original TopTen™ method; the analysis was programmed for a full scan recorded between 375-1575 Da at 60,000 resolution, and a MS/MS scan at resolution 15,000 to generate product ion spectra to determine amino acid sequence in consecutive instrument scans of the fifteen most abundant peaks in the spectrum. The AGC Target ion number was set at 3e6 ions for full scan and 2e5 ions for MS2 mode. Maximum ion injection time was set at 50 ms for full scan and 55 ms for MS2 mode. Micro scan number was set at 1 for both full scan and MS2 scan. The HCD fragmentation energy (N)CE/stepped NCE was set to 28 and an isolation window of 4 m/z. Singly charged ions were excluded from MS2. Dynamic exclusion was enabled with a repeat count of 1 within 15 seconds and to exclude isotopes. A Siloxane background peak at 445.12003 was used as the internal lock mass. HeLa protein digest standard is used to evaluate the integrity and the performance of the columns and mass spectrometer. If the number of protein IDs from the HeLa standard falls below 2700, the instrument is cleaned, and new columns are installed.

Data processing and analysis. All MS/MS samples were analyzed using Sequest (Thermo Fisher Scientific, San Jose, CA, USA; version IseNode in Proteome Discoverer 2.4.0.305). Sequest was set up to search Homo sapiens (NcbiAV TaxID=9606) (v2017-10-30) assuming the digestion enzyme trypsin. Sequest was searched with a fragment ion mass tolerance of 0.020 Da and a parent ion tolerance of 10.0 ppm. Carbamidomethyl of cysteine was specified in Sequest as a fixed modification. Met-loss of methionine, met-loss+Acetyl of methionine, oxidation of methionine and acetyl of the N-terminus were specified in Sequest as variable modifications. Protein identifications were accepted if they could be established with less than 1.0% false discovery and contained at least 2 identified peptides.

### TurboID-Bait and LC-MS/MS

Construction of the pcDNA3-V5-TurboID-CDK9 plasmid. To build the TurboID-bait assay, we first constructed a V5-TurboID-tagged CDK9 plasmid (pDL2518) through Gibson assembly method. The template plasmids HA-CDK9 (a gift from Andrew Rice, plasmid #28102) and V5-TurboID-NES_pCDNA3 (a gift from Alice Ting, plasmid #107169) were purchased from Addgene. The primer pair (5′-cagtctgcggtctgccgaaaagctgcagATGGCAAAGCAGTACGACTCGGTGGAGT-3′ and 5′-cagggtcaggcgctccaggggaggcagTCAGAAGACGCGCTCAAACTCCGTCTGGT-3′) and the template HA-CDK9 were used to clone the CDK9 fragment. The two primer pairs: pair-1 (5′-ACCAGACGGAGTTTGAGCGCGTCTTCTGActgcctcccctggagcgcctgaccctg-3′ and 5′-caatgatgagcacttttaaagttctgctatgtggcgcggtattatcccgtattgac-3′); pair-2 (5′-gtcaatacgggataataccgcgccacatagcagaactttaaaagtgctcatcattg-3′ and 5′-ACTCCACCGAGTCGTACTGCTTTGCCATctgcagcttttcggcagaccgcagactg-3′) and the template V5-TurboID-NES_pCDNA3 were used to clone the V5-TurbolD fragment. The PCR fragments were assembled using NEBuilder® HiFi DNAAssembly Master Mix (Cat. No. E2621, NEB, Ipswich, MA, USA). DNA sequences in all these plasmids were authenticated by automatic sequencing.

TurboID-bait assay. 293T (6×10⁵ for WB detection and 1.2×10⁶ for MS) cells were transfected with plasmid V5-TurboID-CDK9 using Lipofectamine 2000 as previously described (Lv et al., 2021). After 36 h, the transfected cells were pretreated with 10 M MG132 for 1 h and followed up with co-treatment of DMSO or compound 955 and 50 M D-Biotin at 37° C. for 6 h. Then the cells were harvested and lysed in 1×PBS buffer (Cat. #D1408, Sigma-Aldrich). After centrifugation at 20,000×g for 60 min at 4° C., the supernatant soluble fraction was used for further analysis. The same amount of protein was used for the enrichment of biotinylated proteins using 100 μl Pierce™ streptavidin agarose beads (Cat. #20353, ThermoFisher) overnight at 4° C. The beads were further washed 3 times with PBS and deionized water respectively for LC-MS/MS analysis or boiled at 95° C. for 5 min in 100 ul 1× Laemmli SDS-Sample buffer (Cat #. BP-110R, Boston BioProducts) for immunoblot analysis.

Sample preparation and LC-MS/MS analysis. Protein samples were reduced, alkylated, and digested on-bead as mentioned above. Tryptic peptides were then separated by reverse phase XSelect CSH C18 2.5 um resin (Waters) on an in-line 150×0.075 mm column using an UltiMate 3000 RSLCnano system (Thermo). Peptides were eluted using a 60 min gradient from 98:2 to 65:35 buffer A:B ratio (Buffer A: 0.1% formic acid, 0.5% acetonitrile; Buffer B: 0.1% formic acid, 99.9% acetonitrile). Eluted peptides were ionized by electrospray (2.4 kV) followed by mass spectrometric analysis on an Orbitrap Eclipse Tribrid mass spectrometer (Thermo). MS data were acquired using the FTMS analyzer in profile mode at a resolution of 120,000 over a range of 375 to 1200 m/z. Following HCD activation, MS/MS data were acquired using the ion trap analyzer in centroid mode and normal mass range with a normalized collision energy of 30%. Proteins were identified by database search using MaxQuant (Max Planck Institute) with a parent ion tolerance of 2.5 ppm and a fragment ion tolerance of 0.5 Da. Scaffold Q+S (Proteome Software) was used to verify MS/MS based peptide and protein identifications. Protein identifications were accepted if they could be established with less than 1.0% false discovery and contained at least 2 identified peptides. Protein probabilities were assigned by the Protein Prophet algorithm (Nesvizhskii et al., 2003) and to perform reporter ion-based statistical analysis.

### TMT-Based Proteomics

Sample Preparation and LC-MS/MS analysis. Total protein from cell pellets was reduced, alkylated, and purified by chloroform/methanol extraction prior to digestion with sequencing grade modified porcine trypsin (Cat. No. V5111, Promega). Tryptic peptides were labeled using tandem mass tag isobaric labeling reagents (Cat. No. A34808, ThermoFisher) following the manufacturer's instructions and combined into one 11-plex sample group. The labeled peptide multiplex was separated into 46 fractions on a 100×1.0 mm Acquity BEH C18 column (Waters) using an UltiMate 3000 UHPLC system (Thermo) with a 50 min gradient from 99:1 to 60:40 bufferA:B (Buffer A: 0.1% formic acid, 0.5% acetonitrile; Buffer B: 0.1% formic acid, 99.9% acetonitrile. Both buffers adjusted to pH 10 with ammonium hydroxide for offline separation) ratio under basic pH conditions, and then consolidated into 18 super-fractions. Each super-fraction was then further separated by reverse phase XSelect CSH C18 2.5 um resin (Waters) on an in-line 150×0.075 mm column using an UltiMate 3000 RSLCnano system (Thermo). Peptides were eluted using a 75 min gradient from 98:2 to 60:40 buffer A:B ratio. Eluted peptides were ionized by electrospray (2.4 kV) followed by mass spectrometric analysis on an Orbitrap Eclipse Tribrid mass spectrometer (Thermo) using multi-notch MS3 parameters. MS data were acquired using the FTMS analyzer in top-speed profile mode at a resolution of 120,000 over a range of 375 to 1500 m/z. Following CID activation with normalized collision energy of 35.0, MS/MS data were acquired using the ion trap analyzer in centroid mode and normal mass range. Using synchronous precursor selection, up to 10 MS/MS precursors were selected for HCD activation with normalized collision energy of 65.0, followed by acquisition of MS3 reporter ion data using the FTMS analyzer in profile mode at a resolution of 50,000 over a range of 100-500 m/z.

Data processing and analysis. Proteins were identified and MS3 reporter ions quantified using MaxQuant (version 2.0.3.0; Max Planck Institute) against the Homo sapiens UniprotKB database (March 2021) with a parent ion tolerance of 3 ppm, a fragment ion tolerance of 0.5 Da, and a reporter ion tolerance of 0.003 Da. Scaffold Q+S (Proteome Software) was used to verify MS/MS based peptide and protein identifications (protein identifications were accepted if they could be established with less than 1.0% false discovery and contained at least 2 identified peptides; protein probabilities were assigned by the Protein Prophet algorithm (Nesvizhskii et al., 2003) and to perform reporter ion-based statistical analysis. Protein TMT MS3 reporter ion intensity values were assessed for quality using ProteiNorm for a systematic evaluation of normalization methods (Graw et al., 2020). Cyclic loess normalization (Ritchie et al., 2015) was utilized since it had the highest intragroup correlation and the lowest variance amongst the samples. Statistical analysis was performed using Linear Models for Microarray Data (limma) with empirical Bayes (eBayes) smoothing to the standard errors (Ritchie et al., 2015). Proteins with an FDR adjusted p-value<0.05 and a fold change >2 were considered to be significant.

### RNA-Seq

Total RNA was extracted using RNeasy Plus Mini Kit (Qiagen, Cat. No. 74134). The library construction, cluster generation and DNBseq (BGI) sequencing were performed with BGI following the previously reported methods (Senabouth et al., 2020). Raw fastq data were analyzed by using Galaxy (usegalaxy.org/) as described previously (Lv et al., 2018). Human genome (hg38) was used as the reference genome. Differentially expressed gene was analyzed by using DESeq2 based on a false-discovery rate-adjusted q-value (q<0.05). Genes with more than 2-fold change were selected for further analysis.

### Reverse Transcription and Quantitative PCR (RT-qPCR)

Total RNA was extracted as mentioned above. Reverse transcription and quantitative PCR (qPCR) was performed as described in our previous study (Lv et al., 2021). Primers used for measuring gene transcriptional level: GAPDH primers (forward 5′-GACCACTTTGTCAAGCTCATTTC-3′ and reverse 5′-CTCTCTTCCTCTTGTGCTCTTG-3′) were described previously (Zhang et al., 2018b). MCL1 primers are forward 5′-ATCTCTCGGTACCTTCGGGAGC-3′ and reverse 5′-GCTGAAAACATGGATCATCACTCG-3′; MYC primers are forward 5′-GGCTCCTGGCAAAAGGTCA-3′ and reverse 5′-CTGCGTAGTTGTGCTGATGT-3′.

### Expression and Purification of Recombinant KEAP1

The plasmid pet28a-His6-Keap1 for expressing His-KEAP1 was purchased from Addgene (a gift from Yimon Aye, plasmid #62454). To purify His-KEAP1, the plasmid transformed cells were grown in LB broth at 37° C. with shaking until the optical density at 600 nm reached 0.6-0.7. Isopropyl-β-D-thiogalactopyranoside (1 mM) was added to induce protein expression overnight at 16° C. Cell pellet was resuspended in lysis buffer [50 mM Tris-Hcl (pH 7.5), 150 mM NaCl, 1 mM EDTA, 0.05% NP-40, 1 mg/ul Lysozyme, lx protease inhibitor, and 1 mM DTT] and lysed using sonication. The lysate was cleared by centrifugation at 20,000×g for 30 min at 4° C. and purified by using GE Healthcare His GraviTrap™ Kit (Cat. No. 45-002-016, Fisher Scientific). After elution, the proteins were concentrated by using Pierce Protein Concentrators PES, 30K MWCO (Cat. No. 88502, ThermoFisher) and then dialyzed into the buffer containing 50 mM HEPES, 150 mM NaCl, and 0.1 mM EDTA (pH 7.5).

### CRISPR Knockout of KEAP1

To knockout KEAP1, two lentiviral CRISPR knockout plasmids targeting human KEAP1 gene were purchased from Abm (Cat. No. 254911110595). Packaging 293T cells were transfected with KEAP1 single guide RNA (sgRNA) and helper vectors (pMD2.G and psPAX2; Addgene plasmid #12259 and 12260) using Lipofectamine 3000 reagent (Cat. No. L3000015, Thermal Fisher). Medium containing lentiviral particles and 8 g/mL polybrene (Sigma-Aldrich) was used to infect H1299 cells. Infected cells were selected in medium containing 2 g/mL puromycin. The sgRNA target sequences are as follows: sg1: GTACGCCTCCACTGAGTGCA; sg2: TGACAGCACCGTTCATGACG. Single clones were selected by serial dilution.

### KEAP1 Rescue Assay

KEAP1-sg1 knockout H1299 (4×10⁵) cells were transfected with or without plasmid expressing Halo-KEAP1 (Addgene, a gift from Yimon Aye, plasmid #58240) using Lipofectamine 3000 reagent (Cat. No. L3000015, Thermal Fisher). After 24 h, the cells in each well of 6-well plate were divided into two wells and cultured for additional 16 h. The cells were then treated with DMSO or 0.1 M 955 at 37° C. for 6 h, harvested and lysed for WB analysis.

### Gel-Based ABPP Assay

A gel-based ABPP assay was performed as previously described with some modifications (Ward et al., 2019). Briefly, Purified His-KEAP1 (0.2 g) was diluted into 50 μL of PBS and 0.5 μL of either DMSO (vehicle) or indicated compound was added to incubate at 25° C. for 1 h, then the samples were treated with 20 M IA-Alkyne (Cat. No. 7015, Tocris, prepared in DMSO) at 25° C. for 1 h. CuAAC reaction was performed following the protocol from ClickChemistryTools (Cat. No. 1001) and Azide-fluor 488 (Cat. No. 760765, Sigma) was used to react with IA-Alkyne. Protein pellet was dissolved in RIPA buffer and then 20 L of 4× reducing Laemmli SDS sample loading buffer (Cat. No. J63615, Alfa Aesar) was added and heated at 95° C. for 5 min. The samples were resolved using precast 4-20% Tris-glycine gels (Mini-PROTEAN TGX, Cat. No. 4561094, Bio-Rad). Fluorescent imaging was performed on the ChemiDoc MP Imaging System (Bio-Rad). Then the gel was stained using the silver stain kit following the instructions from Thermo Fisher (Cat. No. 24612) and imaged using ChemiDoc.

### NanoBRET Ternary Complex Formation Assay

CMV HiBit (Cat. No. CS1956B03) was purchased from Promega. Plasmid HaloTag-KEAP1 (a gift from Yimon Aye, plasmid #58240) was purchased from Addgene. HiBit-CDK9 were constructed through the Gibson assembly method. The primer pair (5′-TGGCTCGAGCGGTGGGAATTCTGGTatggcaaagcagtacgactcggtggag-3′ and 5′-TCTTCCGCTAGCTCCACCGGATCCTCCTCAgaagacgcgctcaaactccgtctggt-3′) and the template HA-CDK9 (a gift from Andrew Rice, plasmid #28102) was used to clone the CDK9 fragment. The primer pair (5′-accagacggagtttgagcgcgtcttcTGAGGAGGATCCGGTGGAGCTAGCGGAAGA-3′ and 5′-ctccaccgagtcgtactgcttcgccatACCAGAATTCCCACCGCTCGAGCCA-3′) and pBit3.1—N(Cat. No. N2361, Promega) were used to clone the vector-containing fragment. The PCR fragments were assembled using NEBuilder® HiFi DNAAssembly Master Mix. DNA sequences in all these plasmids were authenticated by automatic sequencing. 293T cells (8×10⁵) were transfected with Lipofectamine 2000 (Life Technologies) and 1 g HaloTag-KEAP1, 10 ng HiBit-CDK9 and 10 ng LgBit. After 24 h, 2×10⁴ transfected cells were seeded into white 96-well tissue culture plates in Gibco™ Opti-MEM™ I Reduced Serum Medium, No Phenol Red (Cat. No. 11-058-021, Fisher) containing 4% FBS with or without HaloTag NanoBRET 618 Ligand (Cat. No. PRN1662, Promega) and incubated overnight at 37° C., 5% CO₂. The following day, serially diluted compounds were added into the medium and plates were incubated at 37° C., 5% CO₂, for 6 h. After treatment, NanoBRET Nano-Glo Substrate (Cat. No. N1662, Promega) was added into the medium, and the contents were mixed by shaking the plate for 30 s before measuring donor and acceptor signals on Biotek plate reader. Dual-filtered luminescence was collected with a 450/50 nm bandpass filter (donor, NanoBiT-CDK9 protein) and a 610-nm longpass filter (acceptor, HaloTag NanoBRET ligand) using an integration time of 0.5 s. mBRET ratios were calculated following the NanoBRET™ Nano-Glo® Detection System (Cat. No. N1662, Promega).

### Quantification and Statistical Analysis

Immunoblot data were quantified using the ImageJ (v 1.53a) software from NIH. Data analysis was performed using GraphPad Prism software (version 9) unless indicated otherwise. The n number for each experiment is indicated in the figure legends. All graphs presented represent the mean±standard deviation of the mean (SD) unless otherwise stated.

### Chemistry Materials and Methods

### General Materials and Methods

DMF and DCM were obtained via a solvent purification system by filtering through two columns packed with activated alumina and 4A molecular sieve, respectively. Water was purified with a Milli-Q Simplicity 185 Water Purification System (Merck Millipore). All other chemicals and solvents obtained from commercial suppliers were used without further purification. PL-Alkyne was obtained from our previous study (Zhang, X.; Zhang, S.; Liu, X.; Wang, Y.; Chang, J.; Zhang, X.; Mackintosh, S. G.; Tackett, A. J.; He, Y.; Lv, D., Oxidation resistance 1 is a novel senolytic target. Aging cell 2018, 17 (4), e12780). Flash chromatography was performed using silica gel (230-400 mesh) as the stationary phase. Reaction progress was monitored by thin-layer chromatography (silica-coated glass plates) and visualized by 256 nm and 365 nm UV light, and/or by LC-MS. 1H NMR spectra were recorded in CDCl₃ or CD₃0D at 600 MHz, and 13C NMR spectrum was recorded at 151 MHz. Chemical shifts 6 are given in ppm using tetramethylsilane as an internal standard. Multiplicities of NMR signals are designated as singlet (s), doublet (d), doublet of doublets (dd), triplet (t), quartet (q), A triplet of doublets (td), A doublet of triplets (dt), multiplet (m), and broad (br). All final compounds for biological testing were of ≥95.0% purity as analyzed by LC-MS, performed on an Advion AVANT LC system with the expression CMS using a Thermo Accucore™ Vanquish™ C18+ UHPLC Column (1.5 μm, 50×2.1 mm) at 40° C. Gradient elution was used for UHPLC with a mobile phase of acetonitrile and water containing 0.1% formic acid. High resolution mass spectra (HRMS) were recorded on an Agilent 6230 Time-of-Flight (TOF) mass spectrometer.

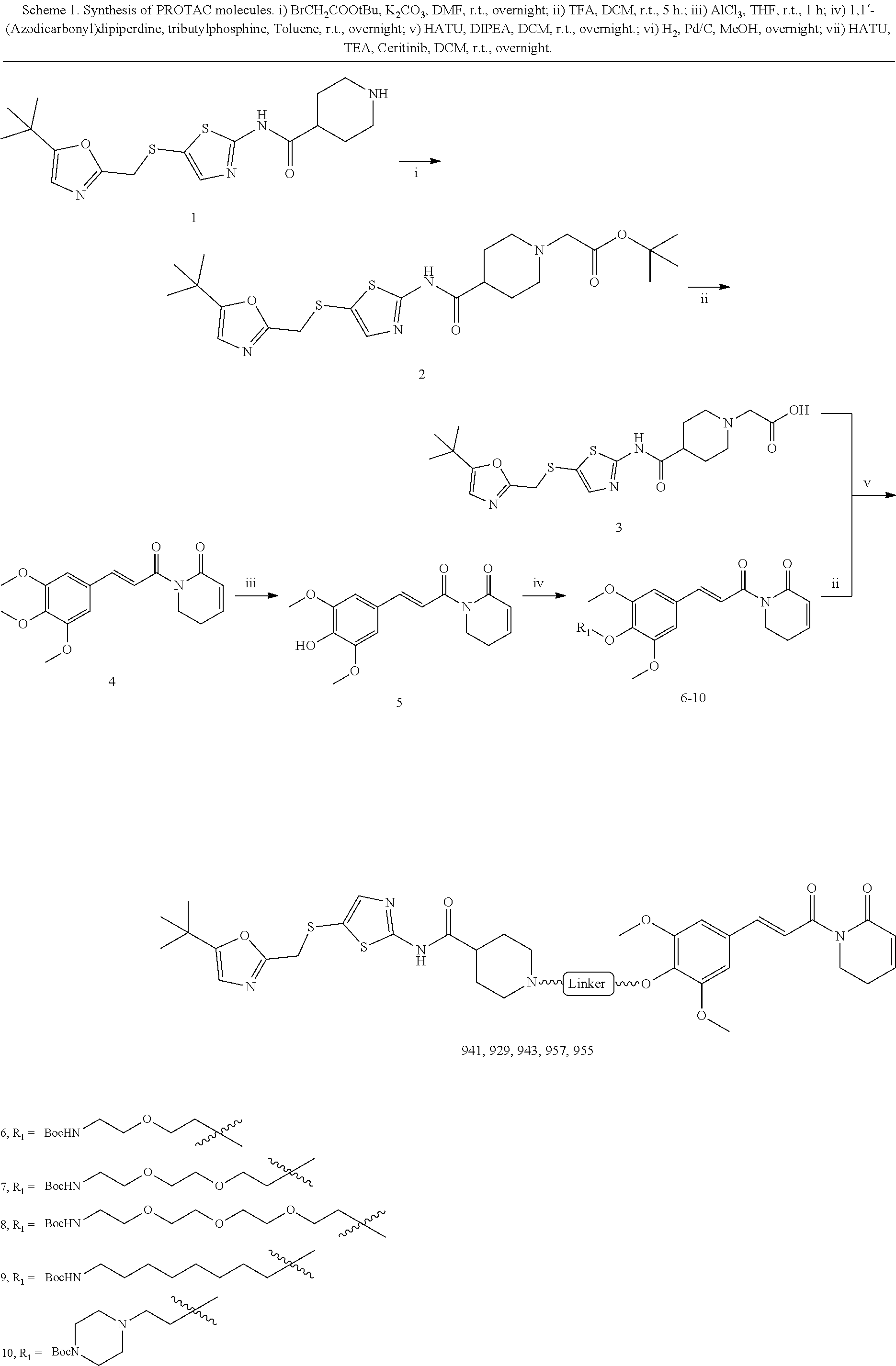

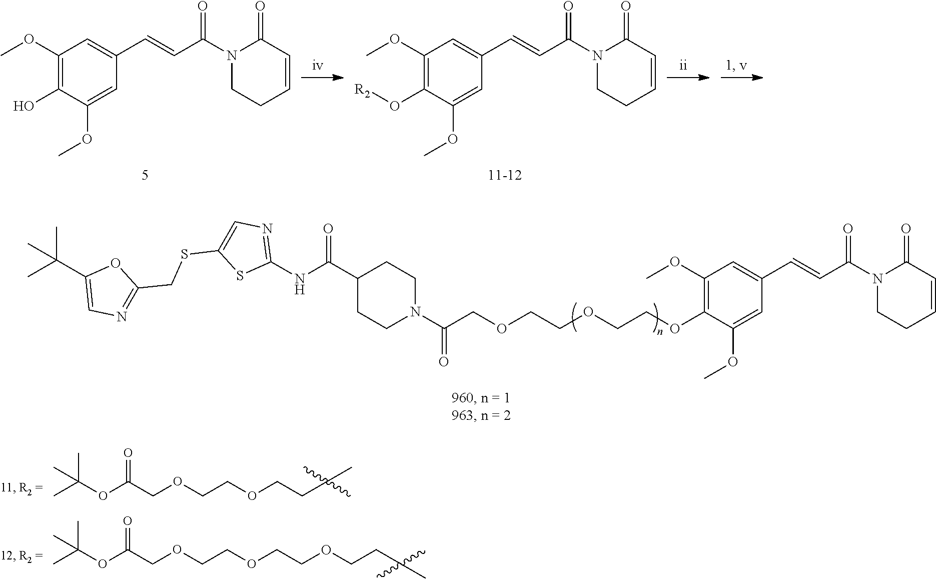

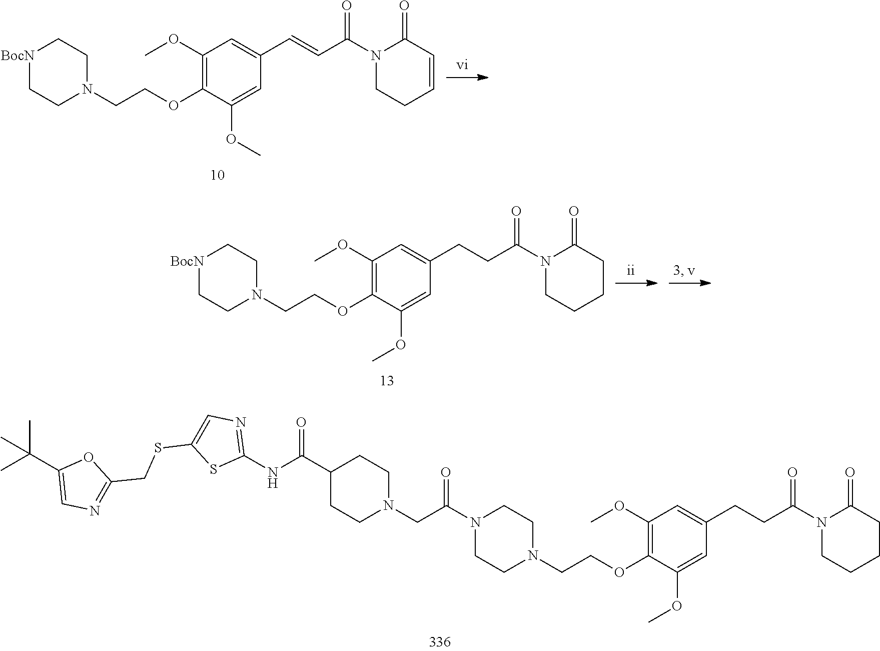 [SMILES: COc1cc(/C=C/C(=O)N2CCC=CC2=O)cc(OC)c1OCCN1CCN(C(=O)OC(C)(C)C)CC1 ; COc1cc(CCC(=O)N2CCCCC2=O)cc(OC)c1OCCN1CCN(C(=O)OC(C)(C)C)CC1 ; COc1cc(CCC(=O)N2CCCCC2=O)cc(OC)c1OCCN1CCN(C(=O)CN2CCC(C(=O)Nc3ncc(SCc4ncc(C(C)(C)C)o4)s3)CC2)CC1]

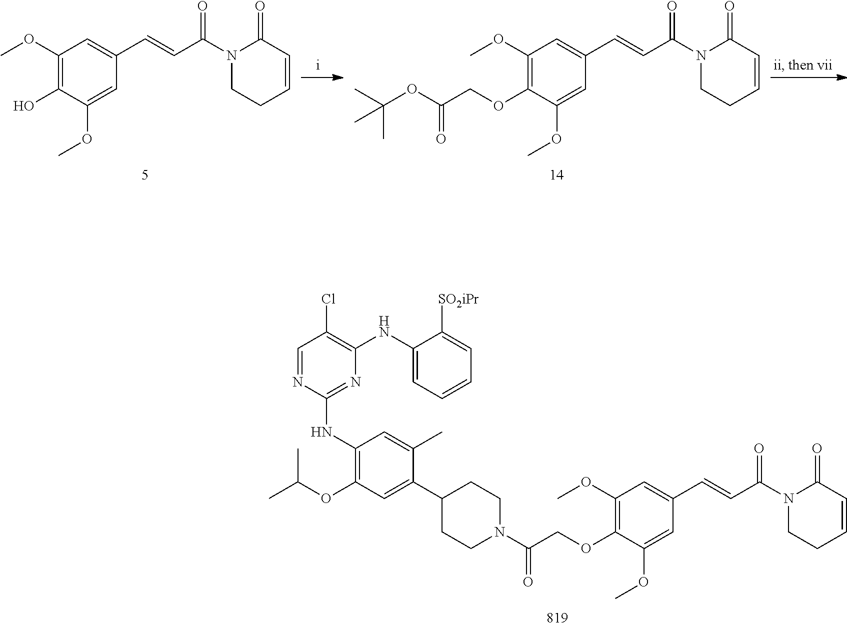

### Preparation of tert-butyl 2-(4-((5-(((5-(tert-butyl)oxazol-2-yl)methyl)thio)thiazol-2-yl)carbamoyl)piperidin-1-yl)acetate (2)

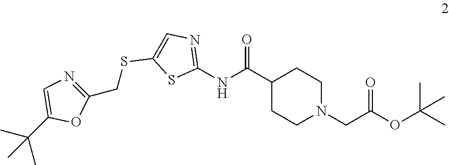 [SMILES: CC(C)(C)OC(=O)CN1CCC(C(=O)Nc2ncc(SCc3ncc(C(C)(C)C)o3)s2)CC1]

To a solution of N-(5-(((5-(tert-butyl)oxazol-2-yl)methyl)thio)thiazol-2-yl)piperidine-4-carboxamide 1 (0.21 g, 0.55 mmol) in DMF (45 mL) was added K₂CO₃ (0.15 g, 1.1 mmol) and tert-Butyl bromoacetate (0.112 g, 0.58 mmol), the reaction mixture was stirred at room temperature overnight. Water was added and the mixture was extracted with EA (30 mL). The organic layers were washed with brine (30 mL*2), dried over anhydrous Na₂SO₄, filtered and concentrated under reduced pressure. The resulting crude was purified by flash chromatography (DCM/MeOH=50/1 to 25/1) to afford the compound 2 (0.586 g, 1.93 mmol, 61% yield) as a white solid. ¹H NMR (600 MHz, Chloroform-d) δ 11.72 (s, 1H), 7.35 (s, 1H), 6.61 (s, 1H), 3.98 (s, 2H), 3.17 (s, 2H), 3.07-2.99 (m, 2H), 2.44-2.38 (m, 1H), 2.30 (td, J=11.6, 2.6 Hz, 2H), 2.01-1.87 (m, 4H), 1.48 (s, 9H), 1.25 (s, 9H). ESI⁺, m/z 495.3 [M+H]⁺.

### Preparation of (E)-1-(3-(4-hydroxy-3,5-dimethoxyphenyl)acryloyl)-5,6-dihydropyridin-2(1H)-one (5)

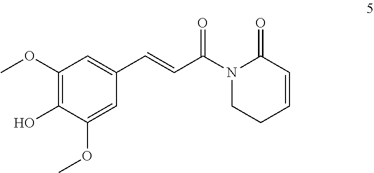 [SMILES: COc1cc(/C=C/C(=O)N2CCC=CC2=O)cc(OC)c1O]

To a solution of (E)-1-(3-(3,4,5-trimethoxyphenyl)acryloyl)-5,6-dihydropyridin-2(1H)-one 4 (1.0 g, 3.15 mmol) in DCM (45 mL) was added AlCl₃ (4.2 g, 31.5 mmol) portion-wise under ice bath, and the resulting reaction mixture was warmed to room temperature and stirred for 1 hr. Saturated NaHCO₃ was added and the mixture was extracted with EA (60 mL*2). The combined organic layers were washed with brine (50 mL), dried over anhydrous Na₂SO₄, filtered and concentrated under reduced pressure to afford a gum. The gum was purified by flash chromatography (DCM/EA=10/1 to 10/2) to afford the compound 5 (0.586 g, 1.93 mmol, 61% yield) as a yellow solid. ¹H NMR (600 MHz, Chloroform-d) δ 7.69 (d, J=15.5 Hz, 1H), 7.41 (d, J=15.5 Hz, 1H), 6.94 (dt, J=9.7, 4.2 Hz, 1H), 6.83 (s, 2H), 6.05 (dt, J=9.7, 1.9 Hz, 1H), 5.75 (s, 1H), 4.04 (t, J=6.5 Hz, 2H), 3.93 (s, 6H), 2.51-2.44 (m, 2H). ESI⁺, m/z 304.1 [M+H]⁺.

### General Procedure A for Compounds 6 to 13

To a solution of (E)-1-(3-(4-hydroxy-3,5-dimethoxyphenyl)acryloyl)-5,6-dihydropyridin-2(1H)-one 5 (1 equiv.) and alcohol (1.1 equiv.) in toluene was added tributylphosphine (1.4 equiv.) and then 1,1′-(Azodicarbonyl)dipiperidine (1.3 equiv.) under ice bath, and the resulting reaction mixture was warmed to room temperature and stirred overnight. Water was added and the mixture was extracted with EA (20 mL*2). The combined organic layers were washed with brine (20 mL), dried over anhydrous Na₂SO₄, filtered and concentrated under reduced pressure. The resulting crude was purified by flash chromatography to afford the desired compound.

### Preparation of tert-butyl (E)-(2-(2-(2,6-dimethoxy-4-(3-oxo-3-(6-oxo-3,6-dihydropyridin-1(2H)-yl)prop-1-en-1-yl)phenoxy)ethoxy)ethyl)carbamate (6)

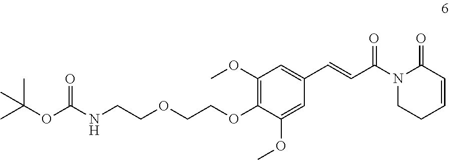 [SMILES: COc1cc(/C=C/C(=O)N2CCC=CC2=O)cc(OC)c1OCCOCCNC(=O)OC(C)(C)C]

84.2 mg (0.17 mmol, 75% yield) was obtained as colorless oil from 5 (70.0 mg, 0.23 mmol) and tert-butyl (2-(2-hydroxyethoxy)ethyl)carbamate (51.3 mg, 0.25 mmol) by using the general procedure A. ¹H NMR (600 MHz, Chloroform-d) δ 7.67 (d, J=15.5 Hz, 1H), 7.43 (d, J=15.6 Hz, 1H), 7.00-6.90 (m, 1H), 6.81 (s, 2H), 6.04 (dt, J=9.7, 1.8 Hz, 1H), 5.27-5.16 (m, 1H), 4.19-4.15 (m, 2H), 4.04 (t, J=6.5 Hz, 2H), 3.89 (s, 6H), 3.77-3.73 (m, 2H), 3.60 (t, J=5.0 Hz, 2H), 3.36-3.30 (m, 2H), 2.52-2.43 (m, 2H), 1.44 (s, 9H).). ESI⁺, m/z 491.2 [M+H]⁺

### Preparation of tert-butyl (E)-(2-(2-(2-(2,6-dimethoxy-4-(3-oxo-3-(6-oxo-3,6-dihydropyridin-1(2H)-yl)prop-1-en-1-yl)phenoxy)ethoxy)ethoxy)ethyl)carbamate (7)

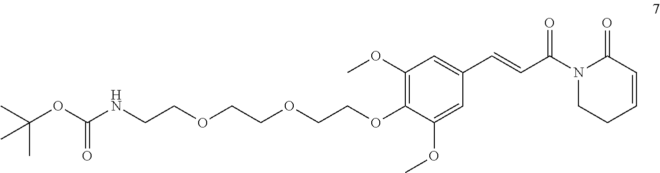 [SMILES: COc1cc(/C=C/C(=O)N2CCC=CC2=O)cc(OC)c1OCCOCCOCCNC(=O)OC(C)(C)C]

89.5 mg (0.17 mmol, 64% yield) was obtained as colorless oil from 5 (80.0 mg, 0.26 mmol) and tert-butyl (2-(2-(2-hydroxyethoxy)ethoxy)ethyl)carbamate (72.3 mg, 0.29 mmol) by using the general procedure A. ¹H NMR (600 MHz, Chloroform-d) δ 7.67 (d, J=15.5 Hz, 1H), 7.42 (d, J=15.5, 1.0 Hz, 1H), 6.98-6.93 (m, 1H), 6.80 (s, 2H), 6.04 (dq, J=9.5, 1.5 Hz, 1H), 5.18-5.10 (m, 1H), 4.22-4.16 (m, 2H), 4.04 (t, J=6.6 Hz, 2H), 3.87 (s, 6H), 3.82-3.78 (m, 2H), 3.73-3.69 (m, 2H), 3.65-3.62 (m, 2H), 3.58-3.53 (m, 2H), 3.36-3.27 (m, 2H), 2.51-2.46 (m, 2H), 1.42 (s, 9H).). ESI⁻, m/z 535.4 [M+H]⁺

### Preparation of tert-butyl (E)-(2-(2-(2-(2-(2,6-dimethoxy-4-(3-oxo-3-(6-oxo-3,6-dihydropyridin-1(2H)-yl)prop-1-en-1-yl)phenoxy)ethoxy)ethoxy)ethoxy)ethyl)carbamate (8)

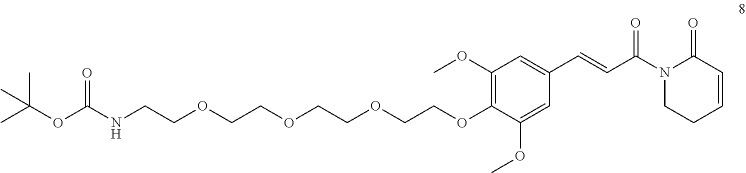 [SMILES: COc1cc(/C=C/C(=O)N2CCC=CC2=O)cc(OC)c1OCCOCCOCCOCCNC(=O)OC(C)(C)C]

65.1 mg (0.11 mmol, 51% yield) was obtained as colorless oil from 5 (65.0 mg, 0.22 mmol) and tert-butyl (2-(2-(2-(2-hydroxyethoxy)ethoxy)ethoxy)ethyl)carbamate (70.4 mg, 0.24 mmol) by using the general procedure A. ¹H NMR (600 MHz, Chloroform-d) δ 7.67 (d, J=15.5 Hz, 1H), 7.42 (d, J=15.5 Hz, 1H), 6.98-6.93 (m, 1H), 6.79 (s, 2H), 6.05 (dt, J=9.7, 1.8 Hz, 1H), 5.17-5.04 (m, 1H), 4.20-4.16 (m, 2H), 4.04 (t, J=6.5 Hz, 2H), 3.87 (s, 6H), 3.84-3.79 (m, 2H), 3.75-3.71 (m, 2H), 3.68-3.60 (m, 6H), 3.54 (t, J=5.2 Hz, 2H), 3.34-3.28 (m, 2H), 2.48 (tdd, J=6.3, 4.1, 1.9 Hz, 2H), 1.43 (s, 9H).). ESI⁺, m/z 579.4 [M+H]⁺

### Preparation of tert-butyl (E)-2-(2,6-dimethoxy-4-(3-oxo-3-(6-oxo-3,6-dihydropyridin-1(2H)-yl)prop-1-en-1-yl)phenoxy)acetate (14)

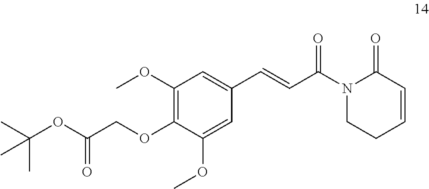 [SMILES: COc1cc(/C=C/C(=O)N2CCC=CC2=O)cc(OC)c1OCC(=O)OC(C)(C)C]

40.2 mg (0.096 mmol, 85% yield) was obtained as colorless oil by using the same procedure as compound 2. ¹H NMR (600 MHz, Chloroform-d) δ 7.67 (d, J=15.5 Hz, 1H), 7.42 (d, J=15.5 Hz, 1H), 6.95 (d, J=9.8 Hz, 1H), 6.79 (s, 2H), 6.05 (dd, J=9.7, 1.9 Hz, 1H), 4.59 (s, 2H), 4.04 (t, J=6.5 Hz, 2H), 3.87 (s, 6H), 2.50-2.45 (m, 2H), 1.47 (s, 9H). ESI⁺, m/z 418.3 [M+H]⁺.

### Preparation of tert-butyl (E)-2-(2-(2-(2,6-dimethoxy-4-(3-oxo-3-(6-oxo-3,6-dihydropyridin-1(2H)-yl)prop-1-en-1-yl)phenoxy)ethoxy)ethoxy)acetate (11)

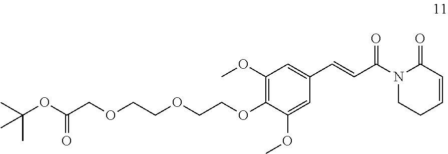 [SMILES: COc1cc(/C=C/C(=O)N2CCC=CC2=O)cc(OC)c1OCCOCCOCC(=O)OC(C)(C)C]

32.2 mg (0.064 mmol, 64% yield) was obtained as colorless oil from 5 (30 mg, 0.10 mmol) and tert-butyl 2-(2-(2-hydroxyethoxy)ethoxy)acetate (24.2 mg, 0.11 mmol) by using the general procedure A. ¹H NMR (600 MHz, Chloroform-d) δ 7.67 (d, J=15.6 Hz, 1H), 7.42 (d, J=15.5 Hz, 1H), 6.98-6.93 (m, 1H), 6.79 (s, 2H), 6.05 (dt, J=9.7, 1.8 Hz, 1H), 4.20-4.17 (m, 2H), 4.07-4.01 (m, 4H), 3.87 (s, 6H), 3.84-3.80 (m, 2H), 3.77-3.73 (m, 4H), 2.51-2.46 (m, 2H), 1.47 (s, 9H). ESI⁺, m/z 506.4 [M+H]⁺.

### Preparation of tert-butyl (E)-2-(2-(2-(2-(2,6-dimethoxy-4-(3-oxo-3-(6-oxo-3,6-dihydropyridin-1(2H)-yl)prop-1-en-1-yl)phenoxy)ethoxy)ethoxy)ethoxy)acetate (12)

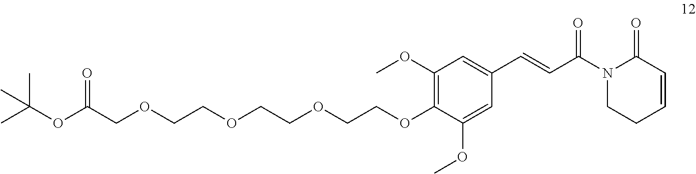 [SMILES: COc1cc(/C=C/C(=O)N2CCC=CC2=O)cc(OC)c1OCCOCCOCCOCC(=O)OC(C)(C)C]

71.5 mg (0.13 mmol, 48% yield) was obtained as colorless oil from 5 (83 mg, 0.27 mmol) and tert-butyl 2-(2-(2-(2-hydroxyethoxy)ethoxy)ethoxy)acetate (79.3 mg, 0.30 mmol) by using the general procedure A. ¹H NMR (600 MHz, Chloroform-d) δ 7.67 (d, J=15.5 Hz, 1H), 7.42 (d, J=15.6 Hz, 1H), 6.99-6.92 (m, 1H), 6.79 (s, 2H), 6.05 (dt, J=9.7, 1.8 Hz, 1H), 4.21-4.15 (m, 2H), 4.06-4.01 (m, 4H), 3.87 (s, 6H), 3.83-3.79 (m, 2H), 3.75-3.67 (m, 8H), 2.51-2.46 (m, 2H), 1.47 (s, 9H). ESI⁺, m/z 550.3 [M+H]⁺.

### Preparation of tert-butyl (E)-(8-(2,6-dimethoxy-4-(3-oxo-3-(6-oxo-3,6-dihydropyridin-1(2H)-yl)prop-1-en-1-yl)phenoxy)octyl)carbamate (9)

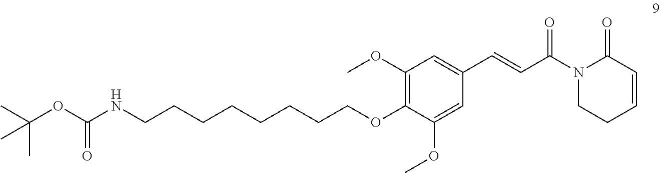 [SMILES: COc1cc(/C=C/C(=O)N2CCC=CC2=O)cc(OC)c1OCCCCCCCCNC(=O)OC(C)(C)C]

31.9 mg (0.060 mmol, 60% yield) was obtained as colorless oil from 5 (30 mg, 0.10 mmol) and tert-butyl 2-(2-(2-(2-hydroxyethoxy)ethoxy)ethoxy)acetate (27.0 mg, 0.11 mmol) by using the general procedure A. ¹H NMR (600 MHz, Chloroform-d) δ 7.68 (d, J=15.5 Hz, 1H), 7.42 (d, J=15.5 Hz, 1H), 6.98-6.92 (m, 1H), 6.80 (s, 2H), 6.05 (dt, J=9.7, 1.8 Hz, 1H), 4.53 (s, 1H), 4.04 (t, J=6.5 Hz, 2H), 3.99 (t, J=6.8 Hz, 2H), 3.87 (s, 6H), 3.10 (q, J=6.8 Hz, 2H), 2.52-2.46 (m, 2H), 1.75-1.71 (m, 2H), 1.50-1.26 (m, 19H). ESI⁺, m/z 531.3 [M+H]⁺.

### Preparation of tert-butyl (E)-4-(2-(2,6-dimethoxy-4-(3-oxo-3-(6-oxo-3,6-dihydropyridin-1(2H)-yl)prop-1-en-1-yl)phenoxy)ethyl)piperazine-1-carboxylate_(10)

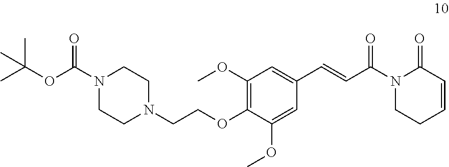 [SMILES: COc1cc(/C=C/C(=O)N2CCC=CC2=O)cc(OC)c1OCCN1CCN(C(=O)OC(C)(C)C)CC1]

180.3 mg (0.35 mmol, 59% yield) was obtained as colorless oil from 5 (180.0 mg, 0.59 mmol) and tert-butyl 4-(2-hydroxyethyl)piperazine-1-carboxylate (149.5 mg, 0.65 mmol) by using the general procedure A. ¹H NMR (600 MHz, Chloroform-d) δ 7.69 (d, J=15.5 Hz, 1H), 7.44 (d, J=15.5 Hz, 1H), 6.99-6.95 (m, 1H), 6.81 (s, 2H), 6.07 (dt, J=9.7, 1.8 Hz, 1H), 4.14 (t, J=5.7 Hz, 2H), 4.07 (t, J=6.5 Hz, 2H), 3.88 (s, 6H), 3.48 (t, J=5.0 Hz, 4H), 2.81 (t, J=5.7 Hz, 2H), 2.59-2.48 (m, 6H), 1.48 (s, 9H). ESI⁺, m/z 516.2 [M+H]⁺.

### Preparation of tert-butyl 4-(2-(2,6-dimethoxy-4-(3-oxo-3-(2-oxopiperidin-1-yl)propyl)phenoxy)ethyl)piperazine-1-carboxylate (13)

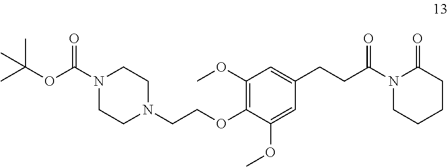 [SMILES: COc1cc(CCC(=O)N2CCCCC2=O)cc(OC)c1OCCN1CCN(C(=O)OC(C)(C)C)CC1]

To a solution of compound 10 (30.3 mg, 0.058 mmol) in 10 ml MeOH was added 10% Pd/C (6 mg, 20 wt % of 10). After purged with nitrogen then hydrogen, the reaction was stirred at room temperature overnight. Filtered it and concentrated under reduced pressure. The resulting crude was purified by flash chromatography to afford the compound 13 (21.3 mg, 0.041 mmol, 71% yield) as colorless oil. ¹H NMR (600 MHz, Chloroform-d) δ 6.44 (s, 2H), 4.06 (t, J=5.8 Hz, 2H), 3.81 (s, 6H), 3.71 (t, J=5.9 Hz, 2H), 3.50-3.44 (m, 4H), 3.25-3.18 (m, 2H), 2.90 (t, J=7.7 Hz, 2H), 2.78 (t, J=5.8 Hz, 2H), 2.59-2.52 (m, 6H), 1.86-1.78 (m, 4H), 1.46 (s, 9H). ESI⁺, m/z 520.3 [M+H]⁺.

### General Procedure B for PROTACs

Boc-protected amine (1 equiv.) and tert-butyl protected acid (1 equiv.) were first converted to their corresponding free amine and acid by reacting with 2 mL trifluoroacetic acid in 2 mL DCM at room temperature for 4 h. Each of them was then concentrated under vacuum to obtain the crude which was used without purification. To a solution of free amin and acid in 4 mL DCM was added DIPEA (10 equiv.) and HATU (1.1 equiv.). The reaction was stirred at room temperature overnight. Water was added and the mixture was extracted with EA (20 mL*2). The combined organic layers were washed with brine (15 mL), dried over anhydrous Na₂SO₄, filtered and concentrated under reduced pressure. The resulting mixture was purified by flash chromatography (DCM/MeOH=30/1 to 20/1) to afford the desired PROTACs.

### Preparation of (E)-N-(5-(((5-(tert-butyl)oxazol-2-yl)methyl)thio)thiazol-2-yl)-1-(2-((2-(2-(2,6-dimethoxy-4-(3-oxo-3-(6-oxo-3,6-dihydropyridin-1(2H)-yl)prop-1-en-1-yl)phenoxy)ethoxy)ethyl)amino)-2-oxoethyl)piperidine-4-carboxamide (941)

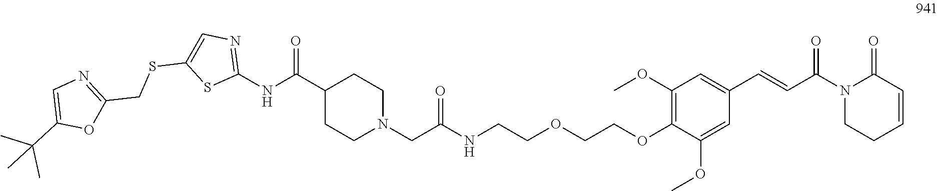 [SMILES: COc1cc(/C=C/C(=O)N2CCC=CC2=O)cc(OC)c1OCCOCCNC(=O)CN1CCC(C(=O)Nc2ncc(SCc3ncc(C(C)(C)C)o3)s2)CC1]

14.1 mg (0.017 mmol, 40% yield) was obtained as colorless gum from 6 (21.0 mg, 0.043 mmol) and 2 (21.3 mg, 0.043 mmol) by using the general procedure B. ¹H NMR (600 MHz, Chloroform-d) δ 10.65 (br, 1H), 7.62 (d, J=15.5 Hz, 1H), 7.47 (t, J=6.1 Hz, 1H), 7.37 (d, J=15.5 Hz, 1H), 7.30 (s, 1H), 6.99-6.92 (m, 1H), 6.77 (s, 2H), 6.59 (s, 1H), 6.07 (dt, J=9.7, 1.8 Hz, 1H), 4.18-4.14 (m, 2H), 4.04 (t, J=6.5 Hz, 2H), 3.96 (s, 2H), 3.86 (s, 6H), 3.80-3.74 (m, 2H), 3.64 (t, J=5.1 Hz, 2H), 3.54-3.48 (m, 2H), 3.04 (s, 2H), 2.96-2.88 (m, 2H), 2.51-2.47 (m, 2H), 2.42-2.34 (m, 1H), 2.28-2.18 (m, 2H), 1.89-1.83 (m, 4H), 1.25 (s, 9H). ¹³C NMR (151 MHz, CDCl₃) δ 172.59, 170.23, 168.87, 166.03, 161.88, 161.78, 158.84, 153.34, 145.65, 144.29, 143.54, 138.81, 130.79, 125.79, 121.29, 121.02, 120.11, 105.47, 77.24, 77.03, 76.82, 72.39, 70.22, 69.86, 61.67, 56.19, 53.13, 41.84, 41.70, 38.92, 34.96, 31.59, 28.57, 28.32, 24.81. MS (ESI); m/z: [M+H]⁺ calcd for C₃₉H₅₁N₆O₉S₂⁺: 811.3153, found 811.3105.

### Preparation of (E)-N-(5-(((5-(tert-butyl)oxazol-2-yl)methyl)thio)thiazol-2-yl)-1-(2-((2-(2-(2-(2,6-dimethoxy-4-(3-oxo-3-(6-oxo-3,6-dihydropyridin-1(2H)-yl)prop-1-en-1-yl)phenoxy)ethoxy)ethoxy)ethyl)amino)-2-oxoethyl)piperidine-4-carboxamide (929)

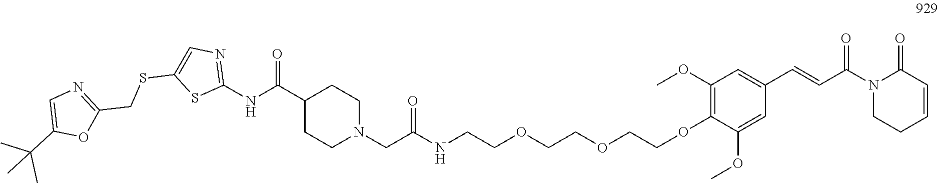 [SMILES: COc1cc(/C=C/C(=O)N2CCC=CC2=O)cc(OC)c1OCCOCCOCCNC(=O)CN1CCC(C(=O)Nc2ncc(SCc3ncc(C(C)(C)C)o3)s2)CC1]

12.1 mg (0.014 mmol, 42% yield) was obtained as colorless gum from 7 (18 mg, 0.034 mmol) and 2 (16.8 mg, 0.034 mmol) by using the general procedure B. ¹H NMR (600 MHz, Chloroform-d) δ 10.47 (br, 1H), 7.64 (d, J=15.5 Hz, 1H), 7.45 (t, J=5.7 Hz, 1H), 7.39 (d, J=15.6 Hz, 1H), 7.30 (s, 1H), 6.98-6.93 (m, 1H), 6.78 (s, 2H), 6.58 (s, 1H), 6.07 (dt, J=9.6, 1.8 Hz, 1H), 4.21-4.16 (m, 2H), 4.04 (t, J=6.5 Hz, 2H), 3.95 (s, 2H), 3.86 (s, 6H), 3.82-3.79 (m, 2H), 3.73-3.69 (m, 2H), 3.66-3.63 (m, 2H), 3.58 (t, J=5.2 Hz, 2H), 3.51-3.46 (m, 2H), 3.01 (s, 2H), 2.93-2.88 (m, 2H), 2.51-2.46 (m, 2H), 2.39-2.34 (m, 1H), 2.22-2.16 (m, 2H), 1.91-1.86 (m, 4H), 1.25 (s, 9H). ¹³C NMR (151 MHz, CDCl₃) δ 172.62, 170.32, 168.93, 166.07, 161.80, 158.84, 153.45, 145.68, 144.40, 143.66, 138.90, 130.77, 125.83, 121.24, 121.09, 120.16, 105.52, 72.29, 70.60, 70.38, 70.27, 70.05, 61.72, 56.21, 53.13, 42.05, 41.71, 38.79, 34.97, 31.44, 28.58, 28.50, 24.82. MS (ESI); m/z: [M+H]⁺ calcd for C₄₁H₅₅N₆O₁₀S₂⁺: 855.3416, found 855.3371.

### Preparation of (E)-N-(5-(((5-(tert-butyl)oxazol-2-yl)methyl)thio)thiazol-2-yl)-1-(14-(2,6-dimethoxy-4-(3-oxo-3-(6-oxo-3,6-dihydropyridin-1(2H)-yl)prop-1-en-1-yl)phenoxy)-2-oxo-6,9,12-trioxa-3-azatetradecyl)piperidine-4-carboxamide (943)

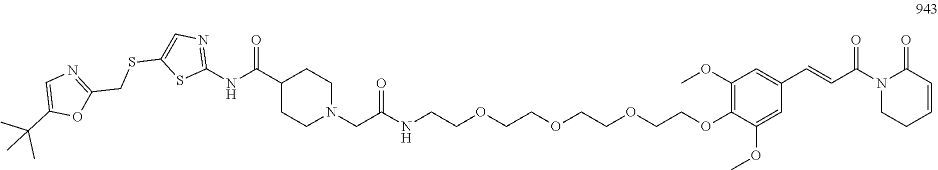 [SMILES: COc1cc(/C=C/C(=O)N2CCC=CC2=O)cc(OC)c1OCCOCCOCCOCCNC(=O)CN1CCC(C(=O)Nc2ncc(SCc3ncc(C(C)(C)C)o3)s2)CC1]

10.8 mg (0.012 mmol, 60% yield) was obtained as colorless gum from 8 (12 mg, 0.02 mmol) and 2 (9.9 mg, 0.02 mmol) by using the general procedure B. ¹H NMR (600 MHz, Chloroform-d) δ 10.70 (br, 1H), 7.65 (d, J=15.5 Hz, 1H), 7.51-7.46 (m, 1H), 7.40 (d, J=15.5 Hz, 1H), 7.29 (s, 1H), 6.99-6.92 (m, 1H), 6.78 (s, 2H), 6.59 (s, 1H), 6.07 (dt, J=9.7, 1.8 Hz, 1H), 4.20-4.16 (m, 2H), 4.04 (t, J=6.5 Hz, 2H), 3.95 (s, 2H), 3.86 (s, 6H), 3.78 (t, J=5.0 Hz, 2H), 3.73-3.61 (m, 8H), 3.57 (t, J=5.1 Hz, 2H), 3.49-3.44 (m, 2H), 3.10-3.00 (m, 2H), 3.00-2.90 (m, 2H), 2.51-2.45 (m, 2H), 2.45-2.37 (m, 1H), 2.32-2.20 (m, 2H), 1.95-1.86 (m, 4H), 1.25 (s, 9H). ¹³C NMR (151 MHz, CDCl₃) δ 172.68, 168.90, 166.00, 161.96, 161.78, 158.85, 153.42, 145.66, 144.27, 143.65, 138.79, 130.79, 125.81, 121.22, 120.93, 120.12, 105.49, 72.32, 70.51, 70.28, 70.05, 56.18, 53.10, 41.70, 38.78, 34.94, 31.44, 28.57, 24.81. MS (ESI); m/z: [M+H]⁺ calcd for C₄₃H₅₉N₆O₁₁S₂+: 899.3678, found 899.3624.

### Preparation of (E)-N-(5-(((5-(tert-butyl)oxazol-2-yl)methyl)thio)thiazol-2-yl)-1-(2-(2-(2-(2,6-dimethoxy-4-(3-oxo-3-(6-oxo-3,6-dihydropyridin-1(2H)-yl)prop-1-en-1-yl)phenoxy)ethoxy)ethoxy)acetyl)piperidine-4-carboxamide (960)

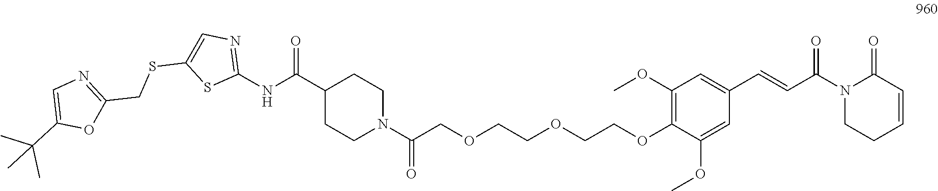 [SMILES: COc1cc(/C=C/C(=O)N2CCC=CC2=O)cc(OC)c1OCCOCCOCC(=O)N1CCC(C(=O)Nc2ncc(SCc3ncc(C(C)(C)C)o3)s2)CC1]

12.0 mg (0.015 mmol, 46% yield) was obtained as colorless gum from 11 (16.0 mg, 0.032 mmol) and 1 (12.2 mg, 0.032 mmol) by using the general procedure B. ¹H NMR (600 MHz, Chloroform-d) δ 10.46 (br, 1H), 7.65 (d, J=15.5 Hz, 1H), 7.38 (d, J=15.5 Hz, 1H), 7.30 (s, 1H), 6.99-6.93 (m, 1H), 6.78 (s, 2H), 6.58 (s, 1H), 6.13 (dt, J=9.7, 1.8 Hz, 1H), 4.55-4.41 (m, 1H), 4.23-4.15 (m, 4H), 4.10-3.91 (m, 5H), 3.86 (s, 6H), 3.80-3.76 (m, 2H), 3.75-3.65 (m, 4H), 3.04-2.95 (m, 1H), 2.82-2.72 (m, 1H), 2.66-2.57 (m, 1H), 2.53-2.46 (m, 2H), 1.94-1.83 (m, 2H), 1.83-1.71 (m, 2H), 1.25 (s, 9H). ¹³C NMR (151 MHz, CDCl₃) δ 171.96, 168.92, 167.79, 166.25, 161.79, 161.59, 158.82, 153.42, 145.80, 144.47, 143.67, 139.17, 130.54, 125.79, 121.19, 121.12, 120.13, 105.47, 72.39, 70.88, 70.69, 70.65, 70.50, 56.20, 44.06, 42.39, 41.75, 41.05, 34.96, 31.44, 28.57, 24.81. MS (ESI); m/z: [M+H]⁺ calcd for C₃₉H₅₀N₅O₁₀S₂⁺: 812.2994, found 812.2946.

### Preparation of (E)-N-(5-(((5-(tert-butyl)oxazol-2-yl)methyl)thio)thiazol-2-yl)-1-(2-(2-(2-(2-(2,6-dimethoxy-4-(3-oxo-3-(6-oxo-3,6-dihydropyridin-1(2H)-yl)prop-1-en-1-yl)phenoxy)ethoxy)ethoxy)ethoxy)acetyl)piperidine-4-carboxamide (963)

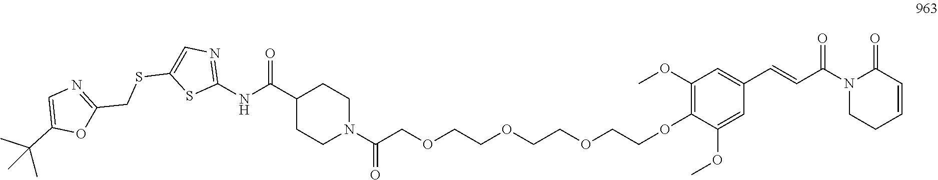 [SMILES: COc1cc(/C=C/C(=O)N2CCC=CC2=O)cc(OC)c1OCCOCCOCCOCC(=O)N1CCC(C(=O)Nc2ncc(SCc3ncc(C(C)(C)C)o3)s2)CC1]

12.2 mg (0.014 mmol, 53% yield) was obtained as colorless gum from 12 (15.0 mg, 0.027 mmol) and 1 (10.3 mg, 0.027 mmol) by using the general procedure B. ¹H NMR (600 MHz, Chloroform-d) δ 10.41 (br, 1H), 7.61 (d, J=15.5 Hz, 1H), 7.29 (s, 1H), 7.25 (d, J=15.5 Hz, 1H), 6.98 (d, J=9.7 Hz, 1H), 6.77 (s, 2H), 6.58 (s, 1H), 6.18 (d, J=9.7 Hz, 1H), 4.25-4.13 (m, 4H), 4.06-3.97 (m, 2H), 3.93 (s, 2H), 3.88 (s, 6H), 3.81-3.67 (m, 11H), 3.62-3.55 (m, 1H), 3.07-2.99 (m, 1H), 2.67-2.59 (m, 2H), 2.54-2.48 (m, 2H), 1.90-1.75 (m, 4H), 1.25 (s, 9H). ¹³C NMR (151 MHz, CDCl₃) δ 172.25, 169.06, 167.94, 166.99, 161.91, 161.55, 159.06, 153.50, 146.26, 145.08, 143.05, 137.28, 131.87, 125.82, 122.53, 120.82, 120.22, 105.07, 71.64, 69.87, 69.69, 69.62, 69.35, 69.17, 56.39, 43.03, 42.11, 41.29, 40.70, 35.11, 31.56, 28.69, 24.90. MS (ESI); m/z: [M+H]⁺ calcd for C₄₁H₅₄N₅O₁₁S₂⁺: 856.3256, found 856.3203.

### Preparation of (E)-N-(5-(((5-(tert-butyl)oxazol-2-yl)methyl)thio)thiazol-2-yl)-1-(2-((8-(2,6-dimethoxy-4-(3-oxo-3-(6-oxo-3,6-dihydropyridin-1(2H)-yl)prop-1-en-1-yl)phenoxy)octyl)amino)-2-oxoethyl)piperidine-4-carboxamide (957)

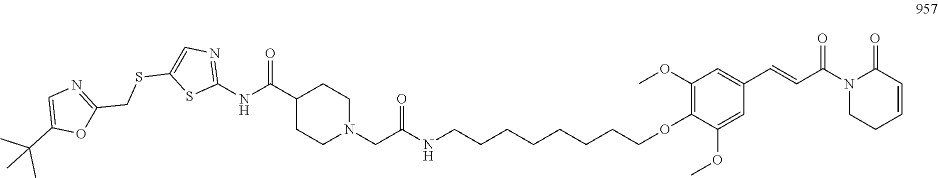 [SMILES: COc1cc(/C=C/C(=O)N2CCC=CC2=O)cc(OC)c1OCCCCCCCCNC(=O)CN1CCC(C(=O)Nc2ncc(SCc3ncc(C(C)(C)C)o3)s2)CC1]

10.7 mg (0.013 mmol, 55% yield) was obtained as colorless gum from 9 (12.0 mg, 0.023 mmol) and 2 (11.4 mg, 0.023 mmol) by using the general procedure B. ¹H NMR (600 MHz, Chloroform-d) δ 10.21 (br, 1H), 7.66 (d, J=15.5 Hz, 1H), 7.40 (d, J=15.5 Hz, 1H), 7.31 (s, 1H), 7.14 (s, 1H), 6.99-6.92 (m, 1H), 6.79 (d, J=5.5 Hz, 2H), 6.59 (s, 1H), 6.06 (dt, J=9.7, 1.8 Hz, 1H), 4.04 (t, J=6.5 Hz, 2H), 3.99 (t, J=6.7 Hz, 2H), 3.95 (s, 2H), 3.86 (s, 6H), 3.30-3.22 (m, 2H), 3.09-2.90 (m, 4H), 2.54-2.46 (m, 2H), 2.46-2.35 (m, 1H), 2.34-2.24 (m, 2H), 1.98-1.85 (m, 4H), 1.77-1.69 (m, 2H), 1.57-1.39 (m, 4H), 1.38-1.29 (m, 6H), 1.26 (s, 9H). ¹³C NMR (151 MHz, CDCl₃) δ 168.97, 165.97, 161.82, 161.54, 158.82, 153.60, 145.59, 144.46, 143.92, 139.44, 130.36, 125.84, 121.27, 120.90, 120.13, 105.64, 73.58, 56.21, 53.14, 41.69, 39.03, 34.96, 31.45, 30.02, 29.63, 29.24, 29.18, 28.58, 26.87, 25.72, 24.82. MS (ESI); m/z: [M+H]⁺ calcd for C₄₃H₅₉N₆O₈S₂⁺: 851.3830, found 851.3784.

### Preparation of (E)-N-(5-(((5-(tert-butyl)oxazol-2-yl)methyl)thio)thiazol-2-yl)-1-(2-(4-(2-(2,6-dimethoxy-4-(3-oxo-3-(6-oxo-3,6-dihydropyridin-1(2H)-yl)prop-1-en-1-yl)phenoxy)ethyl)piperazin-1-yl)-2-oxoethyl)piperidine-4-carboxamide (955)

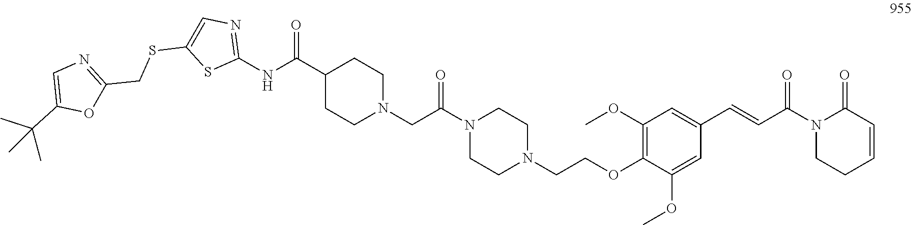 [SMILES: COc1cc(/C=C/C(=O)N2CCC=CC2=O)cc(OC)c1OCCN1CCN(C(=O)CN2CCC(C(=O)Nc3ncc(SCc4ncc(C(C)(C)C)o4)s3)CC2)CC1]

57.3 mg (0.069 mmol, 49% yield) was obtained as white solid from 10 (74.0 mg, 0.14 mmol) and 2 (69.2 mg, 0.14 mmol) by using the general procedure B. ¹H NMR (600 MHz, Methanol-d₄) δ 7.61 (d, J=15.6 Hz, 1H), 7.38 (d, J=15.6 Hz, 1H), 7.31 (s, 1H), 7.07 (d, J=9.7 Hz, 1H), 6.92 (s, 2H), 6.67 (s, 1H), 6.04-5.98 (m, 1H), 4.16-4.09 (m, 2H), 4.02-3.94 (m, 4H), 3.88 (s, 6H), 3.70-3.65 (m, 2H), 3.65-3.58 (m, 2H), 3.24 (s, 2H), 3.02-2.92 (m, 2H), 2.81 (t, J=5.3 Hz, 2H), 2.72-2.66 (m, 2H), 2.66-2.59 (m, 2H), 2.55-2.48 (m, 2H), 2.47-2.42 (m, 1H), 2.21-2.11 (m, 2H), 1.87-1.77 (m, 4H), 1.23 (s, 9H). ¹³C NMR (151 MHz, MeOD) δ 175.24, 170.57, 167.76, 163.46, 163.31, 161.24, 154.97, 148.41, 146.57, 144.18, 139.99, 132.48, 125.89, 122.73, 121.07, 120.68, 106.56, 71.04, 61.28, 58.69, 56.66, 53.85, 46.28, 43.03, 42.65, 35.16, 32.43, 28.89, 25.73. MS (ESI); m/z: [M+H]⁺ calcd for C₄₁H₅₄N7O₈S₂+: 836.3470, found 836.3424.

### Preparation of N-(5-(((5-(tert-butyl)oxazol-2-yl)methyl)thio)thiazol-2-yl)-1-(2-(4-(2-(2,6-dimethoxy-4-(3-oxo-3-(2-oxopiperidin-1-yl)propyl)phenoxy)ethyl)piperazin-1-yl)-2-oxoethyl)piperidine-4-carboxamide (336)

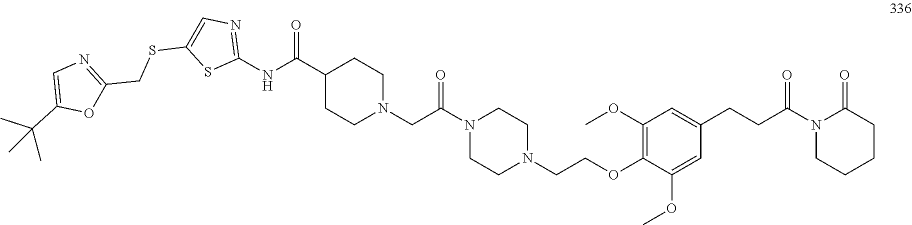 [SMILES: COc1cc(CCC(=O)N2CCCCC2=O)cc(OC)c1OCCN1CCN(C(=O)CN2CCC(C(=O)Nc3ncc(SCc4ncc(C(C)(C)C)o4)s3)CC2)CC1]

8.3 mg (0.001 mmol, 52% yield) was obtained as colorless gum from 13 (10.0 mg, 0.019 mmol) and 2 (9.4 mg, 0.019 mmol) by using the general procedure B. ¹H NMR (600 MHz, Chloroform-d) δ 10.51 (br, 1H), 7.31 (s, 1H), 6.60 (s, 1H), 6.44 (s, 2H), 4.07 (t, J=5.6 Hz, 2H), 3.96 (s, 2H), 3.82 (s, 6H), 3.74-3.64 (m, 6H), 3.30 (s, 2H), 3.24-3.20 (m, 2H), 3.04 (d, J=11.3 Hz, 2H), 2.90 (t, J=7.7 Hz, 2H), 2.80 (t, J=5.5 Hz, 2H), 2.62 (dt, J=22.2, 5.1 Hz, 4H), 2.57-2.52 (m, 2H), 2.42 (s, 1H), 2.28 (s, 2H), 1.95-1.80 (m, 8H), 1.25 (s, 9H). ¹³C NMR (151 MHz, CDCl₃) δ 176.16, 173.54, 172.63, 167.45, 161.83, 161.79, 158.94, 153.17, 144.40, 137.26, 135.04, 121.12, 120.12, 105.44, 69.95, 61.08, 57.87, 56.05, 52.75, 45.37, 44.10, 43.69, 41.39, 34.94, 34.91, 31.56, 31.45, 28.57, 22.44, 20.27. MS (ESI); m/z: [M+H]⁺ calcd for C₄₁H₅₈N₇O₈S₂⁺: 840.3783, found 840.3732.

### Preparation of (E)-1-(3-(4-(2-(4-(4-((5-chloro-4-((2-(isopropylsulfonyl)phenyl)amino)pyrimidin-2-yl)amino)-5-isopropoxy-2-methylphenyl)piperidin-1-yl)-2-oxoethoxy)-3,5-dimethoxyphenyl)acryloyl)-5,6-dihydropyridin-2(1H)-one (819)

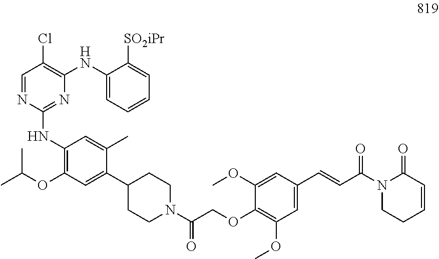

4.3 mg (0.005 mmol, 27% yield) was obtained as colorless gum from 14 (7.6 mg, 0.018 mmol) and Ceritinib (10.2 mg, 0.018 mmol) by using the general procedure B. ¹H NMR (600 MHz, CDCl3) δ 9.50 (s, 1H), 8.58 (d, J=8.3 Hz, 1H), 8.16 (s, 1H), 8.03 (s, 1H), 7.94 (dd, J=8.0, 1.5 Hz, 1H), 7.67 (d, J=15.5 Hz, 1H), 7.65-7.60 (m, 1H), 7.55 (s, 1H), 7.43 (d, J=15.5 Hz, 1H), 7.29-7.24 (m, 1H), 6.98-6.92 (m, 1H), 6.80 (s, 2H), 6.72 (s, 1H), 6.05 (dt, J=9.7, 1.7 Hz, 1H), 4.80 (d, J=13.3 Hz, 1H), 4.72 (d, J=12.0 Hz, 1H), 4.65 (d, J=12.0 Hz, 1H), 4.58-4.50 (m, 2H), 4.05 (t, J=6.5 Hz, 2H), 3.88 (s, 6H), 3.27 (dt, J=13.7, 6.9 Hz, 1H), 3.21 (t, J=12.5 Hz, 1H), 2.98-2.92 (m, 1H), 2.73 (t, J=12.1 Hz, 1H), 2.51-2.46 (m, 2H), 2.20 (s, 3H), 1.84 (t, J=9.5 Hz, 2H), 1.74-1.62 (m, 2H), 1.36 (d, J=6.0 Hz, 6H), 1.32 (d, J=6.9 Hz, 6H). ¹³C NMR (151 MHz, CDCl₃) δ 168.82, 166.51, 165.88, 165.77, 157.46, 155.38, 155.34, 153.34, 145.60, 144.73, 143.55, 138.50, 138.01, 136.79, 134.64, 131.41, 131.29, 127.83, 126.84, 125.82, 124.93, 123.68, 123.12, 121.47, 120.67, 110.83, 105.84, 105.43, 72.11, 71.63, 56.21, 55.46, 46.38, 43.06, 41.66, 38.62, 38.40, 33.46, 32.32, 24.81, 22.32, 22.22, 19.02, 15.38. MS (ESI); m/z: [M+H]⁺ calcd for C₄₆H₅₄ClN₆O₉S₂⁺: 901.3356, found 901.3356.

### References in Example 2

1. Bai, L., Zhou, H., Xu, R., Zhao, Y, Chinnaswamy, K., McEachern, D., Chen, J., Yang, C.-Y., Liu, Z., and Wang, M. (2019). A potent and selective small-molecule degrader of STAT3 achieves complete tumor regression in vivo. Cancer cell 36, 498-511. e417.
2. Bond, M. J., Chu, L., Nalawansha, D. A., Li, K., and Crews, C. M. (2020). Targeted degradation of oncogenic KRASG12C by VHL-recruiting PROTACs. ACS central science 6, 1367-1375.
3. Bondeson, D. P., Mares, A., Smith, I. E., Ko, E., Campos, S., Miah, A. H., Mulholland, K. E., Routly, N., Buckley, D. L., and Gustafson, J. L. (2015). Catalytic in vivo protein knockdown by small-molecule PROTACs. Nature chemical biology 11, 611-617.
4. Branon, T. C., Bosch, J. A., Sanchez, A. D., Udeshi, N. D., Svinkina, T., Carr, S. A., Feldman, J. L., Perrimon, N., and Ting, A. Y. (2018). Efficient proximity labeling in living cells and organisms with TurboID. Nature biotechnology 36, 880-887.
5. Chaudhry, C. (2021). A Mathematical Model for Covalent Proteolysis Targeting Chimeras: Thermodynamics and Kinetics underlying Catalytic Efficiency.
6. Chen, R., Wierda, W. G., Chubb, S., Hawtin, R. E., Fox, J. A., Keating, M. J., Gandhi, V., and Plunkett, W. (2009). Mechanism of action of SNS-032, a novel cyclin-dependent kinase inhibitor, in chronic lymphocytic leukemia. Blood, The Journal of the American Society of Hematology 113, 4637-4645.
7. Gabizon, R., and London, N. (2021). The rise of covalent proteolysis targeting chimeras. Current Opinion in Chemical Biology 62, 24-33.
8. Gadd, M. S., Testa, A., Lucas, X., Chan, K.-H., Chen, W., Lamont, D. J., Zengerle, M., and Ciulli, A. (2017). Structural basis of PROTAC cooperative recognition for selective protein degradation. Nature chemical biology 13, 514-521.
9. Gao, H., Sun, X., and Rao, Y. (2020). PROTAC technology: opportunities and challenges. ACS medicinal chemistry letters 11, 237-240.
10. Graw, S., Tang, J., Zafar, M. K., Byrd, A. K., Bolden, C., Peterson, E. C., and Byrum, S. D. (2020). proteiNorm—a user-friendly tool for normalization and analysis of TMT and label-free protein quantification. ACS omega 5, 25625-25633.
11. Harshbarger, W., Gondi, S., Ficarro, S. B., Hunter, J., Udayakumar, D., Gurbani, D., Singer, W. D., Liu, Y., Li, L., and Marto, J. A. (2017). Structural and biochemical analyses reveal the mechanism of glutathione S-transferase Pi 1 inhibition by the anti-cancer compound piperlongumine. Journal of Biological Chemistry 292, 112-120.
12. He, Y., Khan, S., Huo, Z., Lv, D., Zhang, X., Liu, X., Yuan, Y., Hromas, R., Xu, M., and Zheng, G. (2020). Proteolysis targeting chimeras (PROTACs) are emerging therapeutics for hematologic malignancies. Journal of hematology & oncology 13, 1-24.
13. Henning, N.J., Manford, A. G., Spradlin, J. N., Brittain, S. M., Zhang, E., McKenna, J. M., Tallarico, J. A., Schirle, M., Rape, M., and Nomura, D. K. (2022). Discovery of a covalent FEM1B recruiter for targeted protein degradation applications. Journal of the American Chemical Society.
14. Hines, J., Lartigue, S., Dong, H., Qian, Y., and Crews, C. M. (2019). MDM2-recruiting PROTAC offers superior, synergistic antiproliferative activity via simultaneous degradation of BRD4 and stabilization of p53. Cancer research 79, 251-262.
15. Kawakami, T., Chiba, T., Suzuki, T., Iwai, K., Yamanaka, K., Minato, N., Suzuki, H., Shimbara, N., Hidaka, Y., and Osaka, F. (2001). NEDD8 recruits E2-ubiquitin to SCF E3 ligase. The EMBO journal 20, 4003-4012.
16. Khan, S., Zhang, X., Lv, D., Zhang, Q., He, Y., Zhang, P., Liu, X., Thummuri, D., Yuan, Y., and Wiegand, J. S. (2019). A selective BCL-X L PROTAC degrader achieves safe and potent antitumor activity. Nature medicine 25, 1938-1947.
17. Kido, K., Yamanaka, S., Nakano, S., Motani, K., Shinohara, S., Nozawa, A., Kosako, H., Ito, S., and Sawasaki, T. (2020). AirID, a novel proximity biotinylation enzyme, for analysis of protein-protein interactions. Elife 9, e54983.
18. Kiely-Collins, H., Winter, G. E., and Bernardes, G. J. (2021). The role of reversible and irreversible covalent chemistry in targeted protein degradation. Cell Chemical Biology.
19. Li, L., Zhao, Y., Cao, R., Li, L., Cai, G., Li, J., Qi, X., Chen, S., and Zhang, Z. (2019). Activity-based protein profiling reveals GSTO1 as the covalent target of piperlongumine and a promising target for combination therapy for cancer. Chemical Communications 55, 4407-4410.
20. Li, W., Bengtson, M. H., Ulbrich, A., Matsuda, A., Reddy, V. A., Orth, A., Chanda, S. K., Batalov, S., and Joazeiro, C. A. (2008). Genome-wide and functional annotation of human E3 ubiquitin ligases identifies MULAN, a mitochondrial E3 that regulates the organelle's dynamics and signaling. PloS one 3, e1487.
21. Liu, X., Zhang, X., Lv, D., Yuan, Y., Zheng, G., and Zhou, D. (2020). Assays and technologies for developing proteolysis targeting chimera degraders. Future Medicinal Chemistry 12, 1155-1179.
22. Lu, M., Liu, T., Jiao, Q., Ji, J., Tao, M., Liu, Y., You, Q., and Jiang, Z. (2018). Discovery of a Keap1-dependent peptide PROTAC to knockdown Tau by ubiquitination-proteasome degradation pathway. European journal of medicinal chemistry 146, 251-259.
23. Luo, M., Spradlin, J. N., Boike, L., Tong, B., Brittain, S. M., McKenna, J. M., Tallarico, J. A., Schirle, M., Maimone, T. J., and Nomura, D. K. (2021). Chemoproteomics-enabled discovery of covalent RNF114-based degraders that mimic natural product function. Cell Chemical Biology 28, 559-566. e515.
24. Lv, D.-W., Zhang, K., and Li, R. (2018). Interferon regulatory factor 8 regulates caspase-1 expression to facilitate Epstein-Barr virus reactivation in response to B cell receptor stimulation and chemical induction. PLoS pathogens 14, e1006868.
25. Lv, D., Pal, P., Liu, X., Jia, Y., Thummuri, D., Zhang, P., Hu, W., Pei, J., Zhang, Q., and Zhou, S. (2021). Development of a BCL-xL and BCL-2 dual degrader with improved anti-leukemic activity. Nature Communications 12, 1-14.
26. Mayor-Ruiz, C., Jaeger, M. G., Bauer, S., Brand, M., Sin, C., Hanzl, A., Mueller, A. C., Menche, J., and Winter, G. E. (2019). Plasticity of the Cullin-RING ligase repertoire shapes sensitivity to ligand-induced protein degradation. Molecular cell 75, 849-858. e848.
27. Mullard, A. (2021). Targeted protein degraders crowd into the clinic. Nature reviews. Drug Discovery.
28. Naito, M., Ohoka, N., and Shibata, N. (2019). SNIPERs-Hijacking IAP activity to induce protein degradation. Drug Discovery Today: Technologies 31, 35-42.
29. Nesvizhskii, A. I., Keller, A., Kolker, E., and Aebersold, R. (2003). A statistical model for identifying proteins by tandem mass spectrometry. Analytical chemistry 75, 4646-4658.
30. Ohoka, N., Tsuji, G., Shoda, T., Fujisato, T., Kurihara, M., Demizu, Y., and Naito, M. (2019). Development of small molecule chimeras that recruit AhR E3 ligase to target proteins. ACS chemical biology 14, 2822-2832.
31. Olson, C. M., Jiang, B., Erb, M. A., Liang, Y., Doctor, Z. M., Zhang, Z., Zhang, T., Kwiatkowski, N., Boukhali, M., and Green, J. L. (2018). Pharmacological perturbation of CDK9 using selective CDK9 inhibition or degradation. Nature chemical biology 14, 163-170.
32. Pan, Y., Deng, C., Qiu, Z., Cao, C., and Wu, F. (2021). The Resistance Mechanisms and Treatment Strategies for ALK-Rearranged Non-Small Cell Lung Cancer. Frontiers in Oncology, 3945.
33. Rahaman, M. H., Kumarasiri, M., Mekonnen, L. B., Yu, M., Diab, S., Albrecht, H., Milne, R. W., and Wang, S. (2016). Targeting CDK9: a promising therapeutic opportunity in prostate cancer. Endocrine-related cancer 23, T211-T226.
34. Ritchie, M. E., Phipson, B., Wu, D., Hu, Y., Law, C. W., Shi, W., and Smyth, G. K. (2015). limma powers differential expression analyses for RNA-sequencing and microarray studies. Nucleic acids research 43, e47-e47.
35. Roh, J.-L., Kim, E. H., Park, J. Y., Kim, J. W., Kwon, M., and Lee, B.-H. (2014). Piperlongumine selectively kills cancer cells and increases cisplatin antitumor activity in head and neck cancer. Oncotarget 5, 9227.
36. Sakata, E., Yamaguchi, Y., Miyauchi, Y., Iwai, K., Chiba, T., Saeki, Y., Matsuda, N., Tanaka, K., and Kato, K. (2007). Direct interactions between NEDD8 and ubiquitin E2 conjugating enzymes upregulate cullin-based E3 ligase activity. Nature structural & molecular biology 14, 167-168.
37. Senabouth, A., Andersen, S., Shi, Q., Shi, L., Jiang, F., Zhang, W., Wing, K., Daniszewski, M., Lukowski, S. W., and Hung, S. S. (2020). Comparative performance of the BGI and Illumina sequencing technology for single-cell RNA-sequencing. NAR genomics and bioinformatics 2, lqaa034.
38. Shirasaki, R., Matthews, G. M., Gandolfi, S., de Matos Simoes, R., Buckley, D. L., Vora, J. R., Sievers, Q. L., Bruggenthies, J. B., Dashevsky, O., and Poarch, H. (2021). Functional genomics identify distinct and overlapping genes mediating resistance to different classes of heterobifunctional degraders of oncoproteins. Cell Reports 34, 108532.
39. Spradlin, J. N., Hu, X., Ward, C. C., Brittain, S. M., Jones, M. D., Ou, L., To, M., Proudfoot, A., Ornelas, E., Woldegiorgis, M., et al. (2019). Harnessing the anti-cancer natural product nimbolide for targeted protein degradation. Nat Chem Biol 15, 747-755. 10.1038/s41589-019-0304-8.
40. Suzuki, T., and Yamamoto, M. (2017). Stress-sensing mechanisms and the physiological roles of the Keapl-Nrf2 system during cellular stress. Journal of Biological Chemistry 292, 16817-16824.
41. Tong, B., Luo, M., Xie, Y., Spradlin, J. N., Tallarico, J. A., McKenna, J. M., Schirle, M., Maimone, T. J., and Nomura, D. K. (2020). Bardoxolone conjugation enables targeted protein degradation of BRD4. Scientific reports 10, 1-8.
42. Wang, Y., Chang, J., Liu, X., Zhang, X., Zhang, S., Zhang, X., Zhou, D., and Zheng, G. (2016). Discovery of piperlongumine as a potential novel lead for the development of senolytic agents. Aging (Albany NY) 8, 2915.
43. Ward, C. C., Kleinman, J. I., Brittain, S. M., Lee, P. S., Chung, C. Y. S., Kim, K., Petri, Y., Thomas, J. R., Tallarico, J. A., McKenna, J. M., et al. (2019). Covalent Ligand Screening Uncovers a RNF4 E3 Ligase Recruiter for Targeted Protein Degradation Applications. ACS Chem Biol 14, 2430-2440. 10.1021/acschembio.8b01083.
44. Wei, J., Meng, F., Park, K.-S., Yim, H., Velez, J., Kumar, P., Wang, L., Xie, L., Chen, H., and Shen, Y. (2021). Harnessing the E3 Ligase KEAP1 for Targeted Protein Degradation. Journal of the American Chemical Society 143, 15073-15083.
45. Winter, G. E., Buckley, D. L., Paulk, J., Roberts, J. M., Souza, A., Dhe-Paganon, S., and Bradner, J. E. (2015). Phthalimide conjugation as a strategy for in vivo target protein degradation. Science 348, 1376-1381.
46. Wisniewski, J. R., Zougman, A., Nagaraj, N., and Mann, M. (2009). Universal sample preparation method for proteome analysis. Nature methods 6, 359-362.
47. Zeng, M., Xiong, Y., Safaee, N., Nowak, R. P., Donovan, K. A., Yuan, C. J., Nabet, B., Gero, T. W., Feru, F., and Li, L. (2020). Exploring Targeted Degradation Strategy for Oncogenic KRASG12C. Cell Chemical Biology 27, 19-31. e16.
48. Zhang, C., Han, X.-R., Yang, X., Jiang, B., Liu, J., Xiong, Y., and Jin, J. (2018a). Proteolysis targeting chimeras (PROTACs) of anaplastic lymphoma kinase (ALK). European journal of medicinal chemistry 151, 304-314.
49. Zhang, L., Riley-Gillis, B., Vijay, P., and Shen, Y. (2019a). Acquired resistance to BET-PROTACs (proteolysis-targeting chimeras) caused by genomic alterations in core components of E3 ligase complexes. Molecular cancer therapeutics 18, 1302-1311.
50. Zhang, X., Crowley, V. M., Wucherpfennig, T. G., Dix, M. M., and Cravatt, B. F. (2019b). Electrophilic PROTACs that degrade nuclear proteins by engaging DCAF16. Nature chemical biology 15, 737-746.
51. Zhang, X., Luukkonen, L. M., Eissler, C. L., Crowley, V. M., Yamashita, Y., Schafroth, M. A., Kikuchi, S., Weinstein, D. S., Symons, K. T., and Nordin, B. E. (2021). DCAF11 Supports Targeted Protein Degradation by Electrophilic Proteolysis-Targeting Chimeras. Journal of the American Chemical Society 143, 5141-5149.
52. Zhang, X., Zhang, S., Liu, X., Wang, Y., Chang, J., Zhang, X., Mackintosh, S. G., Tackett, A. J., He, Y., and Lv, D. (2018b). Oxidation resistance 1 is a novel senolytic target. Aging cell 17, e12780.
53. Zoppi, V., Hughes, S. J., Maniaci, C., Testa, A., Gmaschitz, T., Wieshofer, C., Koegl, M., Riching, K. M., Daniels, D. L., and Spallarossa, A. (2018). Iterative design and optimization of initially inactive proteolysis targeting chimeras (PROTACs) identify VZ185 as a potent, fast, and selective von Hippel-Lindau (VHL) based dual degrader probe of BRD9 and BRD7. Journal of medicinal chemistry 62, 699-726.

### REFERENCES

[1]L. Bai, H. Zhou, R. Xu, Y. Zhao, K. Chinnaswamy, D. McEachern, J. Chen, C.-Y. Yang, Z. Liu, M. Wang, Cancer cell 2019, 36, 498-511. e417.
[2]S. Khan, X. Zhang, D. Lv, Q. Zhang, Y. He, P. Zhang, X. Liu, D. Thummuri, Y. Yuan, J. S. Wiegand, Nature medicine 2019, 25, 1938-1947.
[3]D. Lv, P. Pal, X. Liu, Y. Jia, D. Thummuri, P. Zhang, W. Hu, J. Pei, Q. Zhang, S. Zhou, Nature Communications 2021, 12, 1-14.
[4]A. Mullard, Nature reviews. Drug Discovery 2021.
[5]H. Gao, X. Sun, Y. Rao, ACS medicinal chemistry letters 2020, 11, 237-240.
[6]W. Li, M. H. Bengtson, A. Ulbrich, A. Matsuda, V. A. Reddy, A. Orth, S. K. Chanda, S. Batalov, C. A. Joazeiro, PloS one 2008, 3, e1487.
[7]G. E. Winter, D. L. Buckley, J. Paulk, J. M. Roberts, A. Souza, S. Dhe-Paganon, J. E. Bradner, Science 2015, 348, 1376-1381.
[8]D. P. Bondeson, A. Mares, I. E. Smith, E. Ko, S. Campos, A. H. Miah, K. E. Mulholland, N. Routly, D. L. Buckley, J. L. Gustafson, Nature chemical biology 2015, 11, 611-617.
[9]M. Naito, N. Ohoka, N. Shibata, Drug Discovery Today: Technologies 2019, 31, 35-42.
[10]J. Hines, S. Lartigue, H. Dong, Y. Qian, C. M. Crews, Cancer research 2019, 79, 251-262.
[11] aM. J. Bond, L. Chu, D. A. Nalawansha, K. Li, C. Crews, 2020; bM. Zeng, Y. Xiong, N. Safaee, R. P. Nowak, K. A. Donovan, C. J. Yuan, B. Nabet, T. W. Gero, F. Feru, L. Li, Cell Chemical Biology 2020, 27, 19-31. e16.
[12]Y. He, S. Khan, Z. Huo, D. Lv, X. Zhang, X. Liu, Y. Yuan, R. Hromas, M. Xu, G. Zheng, Journal of hematology & oncology 2020, 13, 1-24.
[13] aR. Shirasaki, G. M. Matthews, S. Gandolfi, R. de Matos Simoes, D. L. Buckley, J. R. Vora, Q. L. Sievers, J. B. Bruggenthies, O. Dashevsky, H. Poarch, Cell Reports 2021, 34, 108532; bL. Zhang, B. Riley-Gillis, P. Vijay, Y. Shen, Molecular cancer therapeutics 2019, 18, 1302-1311.
[14] aX. Zhang, S. Zhang, X. Liu, Y. Wang, J. Chang, X. Zhang, S. G. Mackintosh, A. J. Tackett, Y. He, D. Lv, Aging cell 2018, 17, e12780; bY. Wang, J. Chang, X. Liu, X. Zhang, S. Zhang, X. Zhang, D. Zhou, G. Zheng, Aging (Albany NY) 2016, 8, 2915.
[15]J.-L. Roh, E. H. Kim, J. Y. Park, J. W. Kim, M. Kwon, B.-H. Lee, Oncotarget 2014, 5, 9227.
[16]W. Harshbarger, S. Gondi, S. B. Ficarro, J. Hunter, D. Udayakumar, D. Gurbani, W. D. Singer, Y. Liu, L. Li, J. A. Marto, Journal of Biological Chemistry 2017, 292, 112-120.
[17]L. Li, Y. Zhao, R. Cao, L. Li, G. Cai, J. Li, X. Qi, S. Chen, Z. Zhang, Chemical Communications 2019, 55, 4407-4410.
[18]C. M. Olson, B. Jiang, M. A. Erb, Y. Liang, Z. M. Doctor, Z. Zhang, T. Zhang, N. Kwiatkowski, M. Boukhali, J. L. Green, Nature chemical biology 2018, 14, 163-170.
[19] aT. Kawakami, T. Chiba, T. Suzuki, K. Iwai, K. Yamanaka, N. Minato, H. Suzuki, N. Shimbara, Y. Hidaka, F. Osaka, The EMBO journal 2001, 20, 4003-4012; bE. Sakata, Y. Yamaguchi, Y. Miyauchi, K. Iwai, T. Chiba, Y. Saeki, N. Matsuda, K. Tanaka, K. Kato, Nature structural & molecular biology 2007, 14, 167-168.
[20]X. Liu, X. Zhang, D. Lv, Y. Yuan, G. Zheng, D. Zhou, Future Medicinal Chemistry 2020, 12, 1155-1179.
[21]T. C. Branon, J. A. Bosch, A. D. Sanchez, N. D. Udeshi, T. Svinkina, S. A. Carr, J. L. Feldman, N. Perrimon, A. Y. Ting, Nature biotechnology 2018, 36, 880-887.
[22]M. Lu, T. Liu, Q. Jiao, J. Ji, M. Tao, Y. Liu, Q. You, Z. Jiang, European journal of medicinal chemistry 2018, 146, 251-259.
[23] aJ. Wei, F. Meng, K.-S. Park, H. Yim, J. Velez, P. Kumar, L. Wang, L. Xie, H. Chen, Y. Shen, Journal of the American Chemical Society 2021, 143, 15073-15083; bB. Tong, M. Luo, Y. Xie, J. N. Spradlin, J. A. Tallarico, J. M. McKenna, M. Schirle, T. J. Maimone, D. K. Nomura, Scientific reports 2020, 10, 1-8.
[24] aK. Raina, J. Lu, Y. Qian, M. Altieri, D. Gordon, A. M. K. Rossi, J. Wang, X. Chen, H. Dong, K. Siu, Proceedings of the National Academy of Sciences 2016, 113, 7124-7129; bM. Zengerle, K.-H. Chan, A. Ciulli, ACS chemical biology 2015, 10, 1770-1777; cM. S. Gadd, A. Testa, X. Lucas, K.-H. Chan, W. Chen, D. J. Lamont, M. Zengerle, A. Ciulli, Nature chemical biology 2017, 13, 514-521.
[25]T. Suzuki, M. Yamamoto, Journal of Biological Chemistry 2017, 292, 16817-16824.
[26]X. Zhang, V. M. Crowley, T. G. Wucherpfennig, M. M. Dix, B. F. Cravatt, Nature chemical biology 2019, 15, 737-746.
[27]D. P. Bondeson, B. E. Smith, G. M. Burslem, A. D. Buhimschi, J. Hines, S. Jaime-Figueroa, J. Wang, B. D. Hamman, A. Ishchenko, C. M. Crews, Cell chemical biology 2018, 25, 78-87. e75.
[28]M. H. Rahaman, M. Kumarasiri, L. B. Mekonnen, M. Yu, S. Diab, H. Albrecht, R. W. Milne, S. Wang, Endocrine-related cancer 2016, 23, T211-T226.
[29]Y. Pan, C. Deng, Z. Qiu, C. Cao, F. Wu, Frontiers in Oncology 2021, 3945.
[30]C. Zhang, X.-R. Han, X. Yang, B. Jiang, J. Liu, Y. Xiong, J. Jin, European journal of medicinal chemistry 2018, 151, 304-314.
[31] aJ. N. Spradlin, X. Hu, C. C. Ward, S. M. Brittain, M. D. Jones, L. Ou, M. To, A. Proudfoot, E. Ornelas, M. Woldegiorgis, J. A. Olzmann, D. E. Bussiere, J. R. Thomas, J. A. Tallarico, J. M. McKenna, M. Schirle, T. J. Maimone, D. K. Nomura, Nat Chem Biol 2019, 15, 747-755; bC. C. Ward, J. I. Kleinman, S. M. Brittain, P. S. Lee, C. Y. S. Chung, K. Kim, Y. Petri, J. R. Thomas, J. A. Tallarico, J. M. McKenna, M. Schirle, D. K. Nomura, ACS Chem Biol 2019, 14, 2430-2440; cX. Zhang, L. M. Luukkonen, C. L. Eissler, V. M. Crowley, Y. Yamashita, M. A. Schafroth, S. Kikuchi, D. S. Weinstein, K. T. Symons, B. E. Nordin, Journal of the American Chemical Society 2021, 143, 5141-5149.

### INCORPORATION BY REFERENCE

The contents of all references (including literature references, issued patents, published patent applications, and co-pending patent applications) cited throughout this application are hereby expressly incorporated herein in their entireties by reference.

### EMBODIMENTS

1. A compound of Formula (I), or a pharmaceutically acceptable salt, hydrate, solvate, tautomer, or prodrug thereof:

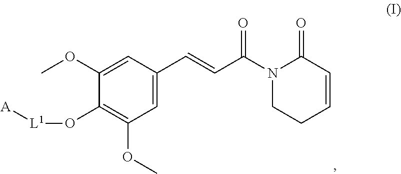

wherein:
A is a kinase inhibitor; and
L¹ is optionally substituted C₁-C₂₀ alkylene, optionally substituted C₁-C₂₀ heteroalkylene, optionally substituted C₁-C₂₀ alkenylene, optionally substituted C₁-C₂₀ heteroalkenylene, optionally substituted C₁-C₂₀ alkynylene, optionally substituted C₁-C₂₀ heteroalkynylene, optionally substituted C₃-C₁₄ carbocyclylene, or optionally substituted 3- to 14-membered heterocyclylene.

2. The compound of embodiment 1, or a pharmaceutically acceptable salt, hydrate, solvate, tautomer, or prodrug thereof, wherein A is a CDK inhibitor.

3. The compound of embodiment 2, or a pharmaceutically acceptable salt, hydrate, solvate, tautomer, or prodrug thereof, wherein A is a CDK9 or CDK10 inhibitor.

4. The compound of any one of embodiments 1-3, or a pharmaceutically acceptable salt, hydrate, solvate, tautomer, or prodrug thereof, wherein A is

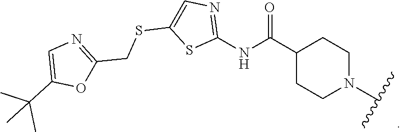 [SMILES: *N1CCC(C(=O)Nc2ncc(SCc3ncc(C(C)(C)C)o3)s2)CC1]

5. The compound of any one of embodiments 1-3, or a pharmaceutically acceptable salt, hydrate, solvate, tautomer, or prodrug thereof, wherein A is AT-7519, atuveciclib, AZD4573, BAY-1251152, CDKI-73, CDKI-73, dinaciclib, flavopiridol, i-CDK9, JSH-150, LDC000067, LY-2857785, NVP-2, RGB-286638, seliciclib, TG02, or zotiraciclib.

6. The compound of embodiment 1, or a pharmaceutically acceptable salt, hydrate, solvate, tautomer, or prodrug thereof, wherein A is an anaplastic lymphoma kinase inhibitor.

7. The compound of embodiment 6, or a pharmaceutically acceptable salt, hydrate, solvate, tautomer, or prodrug thereof, wherein A is

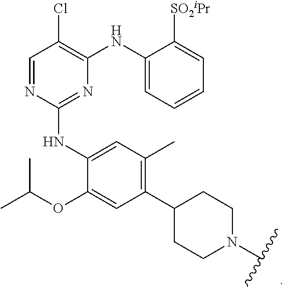

8. The compound of embodiment 6, or a pharmaceutically acceptable salt, hydrate, solvate, tautomer, or prodrug thereof, wherein A is alectinib, AP-26113, ASP-3026, brigatinib, CEP-37440, crizotinib, ensartinib, entrectinib, lorlatinib, NMS-E628, PF-06463922, TSR-011, X-376, or X-396.

9. The compound of any one of embodiments 1-8, or a pharmaceutically acceptable salt, hydrate, solvate, tautomer, or prodrug thereof, wherein L¹ is -(optionally substituted C₁-C₆ alkylene or optionally substituted C₁-C₆ heteroalkylene)₀₋₁-(optionally substituted 3- to 7-membered heterocyclylene)-(optionally substituted C₁-C₆ alkylene or optionally substituted C₁-C₆ heteroalkylene)₀₋₁-.

10. The compound of embodiment 9, or a pharmaceutically acceptable salt, hydrate, solvate, tautomer, or prodrug thereof, wherein L¹ is -(optionally substituted C₂ alkylene)-(optionally substituted monocyclic 6-membered para heterocyclylene)-(optionally substituted C₂ alkylene)-.

11. The compound of any one of embodiments 1-8, or a pharmaceutically acceptable salt, hydrate, solvate, tautomer, or prodrug thereof, wherein L¹ is optionally substituted C₁-C₁₅ alkylene, optionally substituted C₁-C₁₅ heteroalkylene, or optionally substituted 3- to 7-membered heterocyclylene.

12. The compound of any one of embodiments 1-11, or a pharmaceutically acceptable salt, hydrate, solvate, tautomer, or prodrug thereof, wherein L¹ is substituted with a carbonyl.

13. The compound of any one of embodiments 1-8, or a pharmaceutically acceptable salt, hydrate, solvate, tautomer, or prodrug thereof, wherein L¹ is

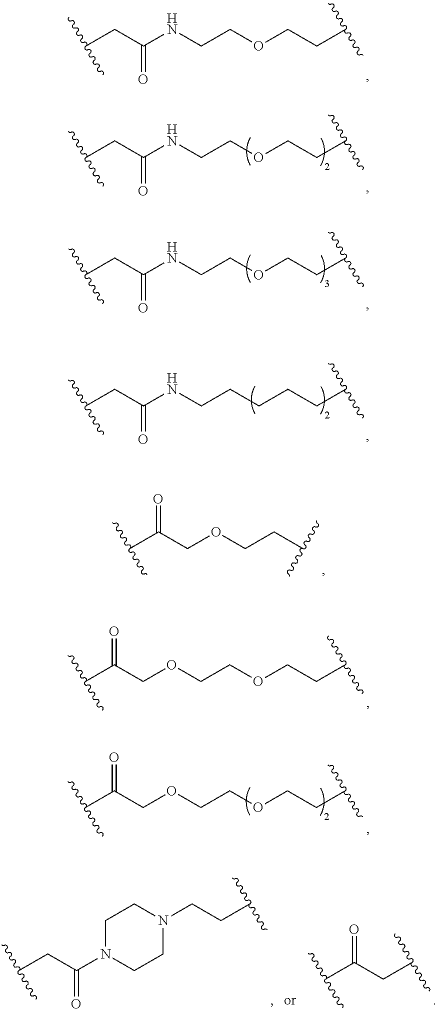

14. The compound of embodiment 1, wherein the compound is

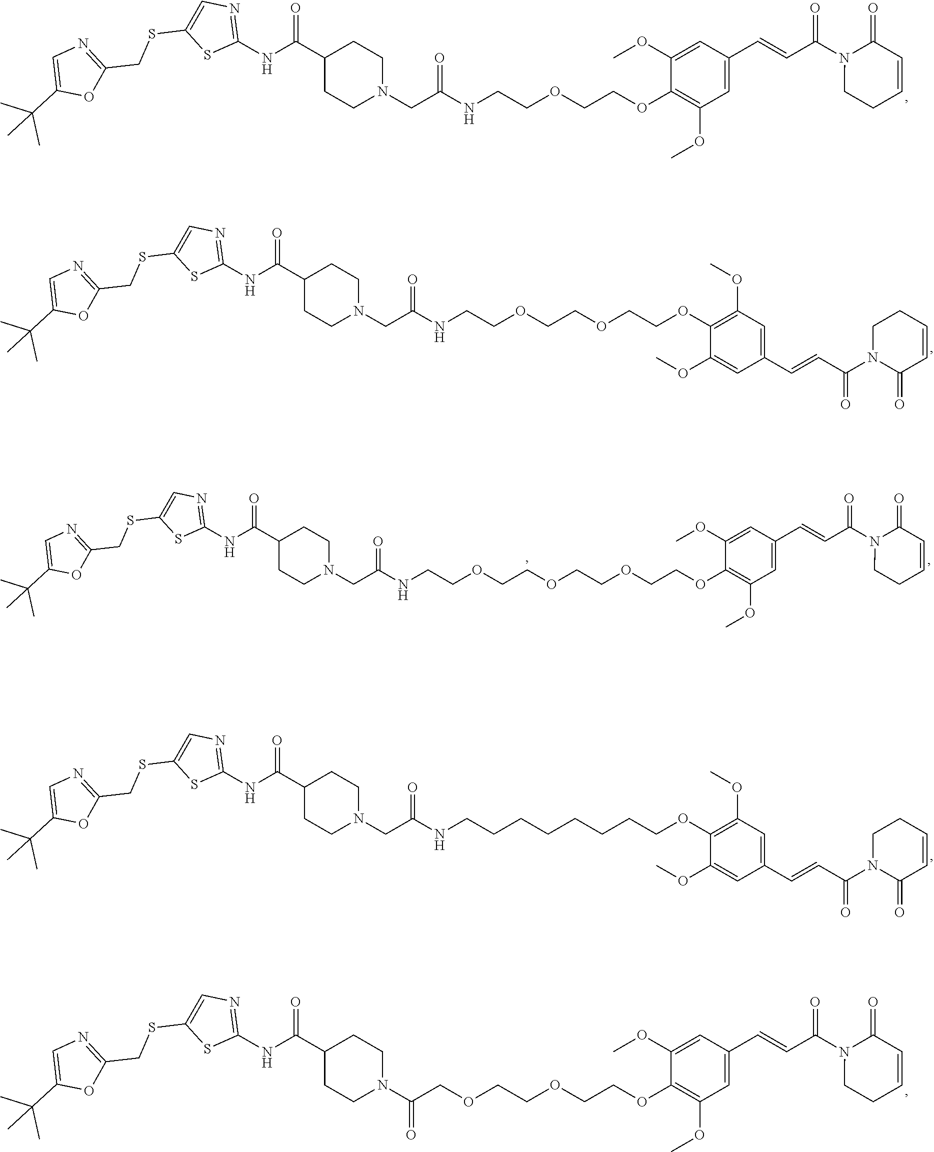 [SMILES: COc1cc(/C=C/C(=O)N2CCC=CC2=O)cc(OC)c1OCCOCCNC(=O)CN1CCC(C(=O)Nc2ncc(SCc3ncc(C(C)(C)C)o3)s2)CC1 ; COc1cc(/C=C/C(=O)N2CCC=CC2=O)cc(OC)c1OCCOCCOCCNC(=O)CN1CCC(C(=O)Nc2ncc(SCc3ncc(C(C)(C)C)o3)s2)CC1 ; COc1cc(/C=C/C(=O)N2CCC=CC2=O)cc(OC)c1OCCOCCOCCOCCNC(=O)CN1CCC(C(=O)Nc2ncc(SCc3ncc(C(C)(C)C)o3)s2)CC1 ; COc1cc(/C=C/C(=O)N2CCC=CC2=O)cc(OC)c1OCCCCCCCCNC(=O)CN1CCC(C(=O)Nc2ncc(SCc3ncc(C(C)(C)C)o3)s2)CC1 ; COc1cc(/C=C/C(=O)N2CCC=CC2=O)cc(OC)c1OCCOCCOCC(=O)N1CCC(C(=O)Nc2ncc(SCc3ncc(C(C)(C)C)o3)s2)CC1]

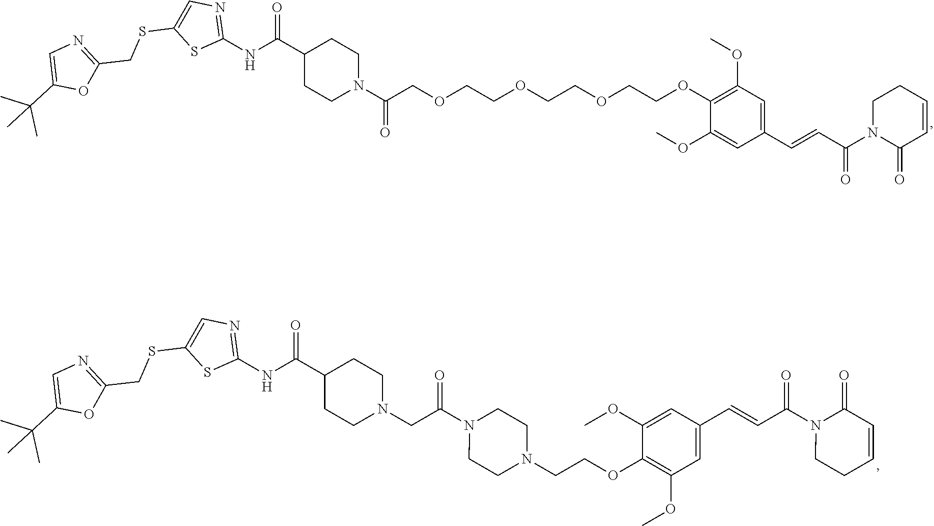 [SMILES: COc1cc(/C=C/C(=O)N2CCC=CC2=O)cc(OC)c1OCCOCCOCCOCC(=O)N1CCC(C(=O)Nc2ncc(SCc3ncc(C(C)(C)C)o3)s2)CC1 ; COc1cc(/C=C/C(=O)N2CCC=CC2=O)cc(OC)c1OCCN1CCN(C(=O)CN2CCC(C(=O)Nc3ncc(SCc4ncc(C(C)(C)C)o4)s3)CC2)CC1]

or a pharmaceutically acceptable salt, hydrate, solvate, tautomer, or prodrug thereof.

15. The compound of embodiment 1, wherein the compound is

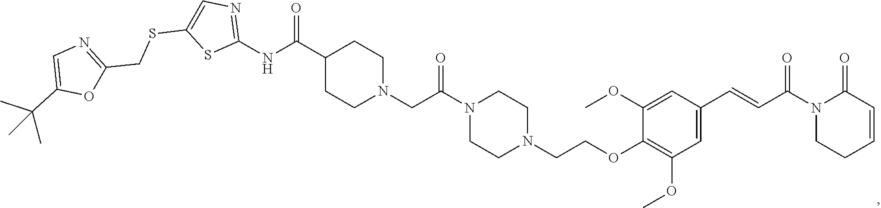 [SMILES: COc1cc(/C=C/C(=O)N2CCC=CC2=O)cc(OC)c1OCCN1CCN(C(=O)CN2CCC(C(=O)Nc3ncc(SCc4ncc(C(C)(C)C)o4)s3)CC2)CC1]

or a pharmaceutically acceptable salt, hydrate, solvate, tautomer, or prodrug thereof.

16. The compound of embodiment 1, wherein the compound is

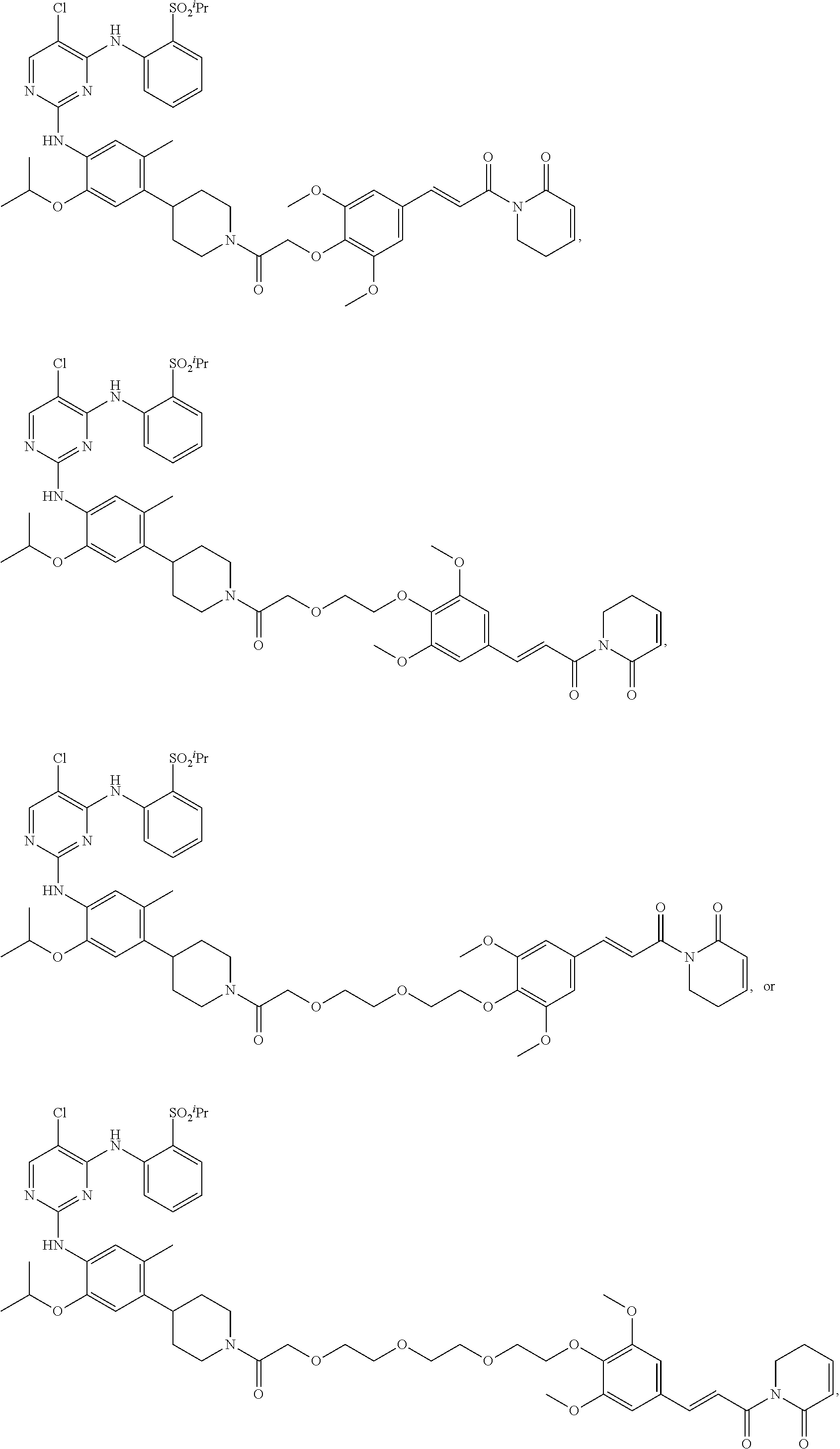

or a pharmaceutically acceptable salt, hydrate, solvate, tautomer, or prodrug thereof.

17. A pharmaceutical composition comprising a compound of any one of embodiments 1-16, or a pharmaceutically acceptable salt, hydrate, solvate, tautomer, or prodrug thereof, and a pharmaceutically acceptable excipient.

18. A method of inhibiting a kinase, the method comprising contacting a kinase with an effective amount of a compound of any one of embodiments 1-16, or a pharmaceutically acceptable salt, hydrate, solvate, tautomer, or prodrug thereof.

19. A method of degrading a kinase, the method comprising contacting a kinase with an effective amount of a compound of any one of embodiments 1-16, or a pharmaceutically acceptable salt, hydrate, solvate, tautomer, or prodrug thereof.

20. The method of embodiment 18 or 19, wherein the contacting is in vitro.

21. The method of embodiment 18 or 19, wherein the contacting in vivo.

22. The method of embodiment 18 or 19, further comprising administering the compound to a subject.

23. The method of any one of embodiments 18-22, wherein the kinase is CDK9 or CDK10.

24. The method of any one of embodiments 18-22, wherein the kinase is anaplastic lymphoma kinase.

25. The method of any one of embodiments 19-24, wherein the degrading is achieved in MOLT4 cells, 293T cells, K562 cells, LNCap cells, 22RV1 cells, PC3 cells, DU145 cells, or NCI-H2228 cells.

26. The method of any one of embodiments 19-25, wherein the degrading is achieved by recruitment of a cullin ring-related ubiquitin E3 ligase.

27. The method of embodiment 26, wherein the cullin ring-related ubiquitin E3 ligase is KEAP1.

28. A method of preventing or treating a disease or disorder in a subject in need thereof, the method comprising administering an effective amount of a compound of any one of embodiments 1-16, or a pharmaceutically acceptable salt, hydrate, solvate, tautomer, or prodrug thereof, or a pharmaceutical composition of embodiment 17.

29. A method of preventing or treating a subject suffering from or susceptible to a disease or disorder, the method comprising administering an effective amount of a compound of any one of embodiments 1-16, or a pharmaceutically acceptable salt, hydrate, solvate, tautomer, or prodrug thereof, or a pharmaceutical composition of embodiment 17.

30. The method of embodiment 28 or 29 of treating the disease or disorder or the subject.

31. The method of any one of embodiments 28-30, wherein the disease or disorder is associated with a CDK.

32. The method of embodiment 31, wherein the CDK is CDK9 or CDK10.

33. The method of any one of embodiments 28-32, wherein the disease or disorder is associated with anaplastic lymphoma kinase.

34. The method of any one of embodiments 28-33, wherein the disease is cancer.

35. The method of embodiment 34, wherein the cancer expresses KEAP1.

36. The method of embodiment 35, wherein the cancer is a leukemia that expresses KEAP1.

37. The method of embodiment 34 or 35, wherein the cancer is a solid tumor or liquid tumor.

38. The method of embodiment 34 or 35, wherein the cancer is lung cancer.

39. The method of embodiment 34 or 35, wherein the cancer is non-small cell lung cancer.

40. The method of embodiment 34 or 35, wherein the cancer is prostate cancer.

41. The method of embodiment 34 or 35, wherein the cancer is bone cancer, brain cancer, breast cancer, cervical cancer, colon cancer, esophageal cancer, head and neck cancer, kidney cancer, liver cancer, melanoma, NUT carcinoma, ovarian cancer, pancreatic cancer, or uterus cancer.

42. The method of embodiment 34 or 35, wherein the cancer is biliary tract cancer, bladder cancer, breast cancer, colorectal cancer, liver cancer, or stomach cancer.

43. The method of embodiment 34 or 35, wherein the cancer is breast cancer, colorectal cancer, esophageal cancer, glioblastoma, inflammatory myofibroblastic tumor, kidney cancer, neuroblastoma, ovarian cancer, pancreatic cancer, rhabdomyosarcoma, salivary gland cancer, or thyroid cancer.

44. The method of embodiment 34 or 35, wherein the cancer is a hematological malignancy.

45. The method of embodiment 34 or 35, wherein the cancer is leukemia.

46. The method of embodiment 34 or 35, wherein the cancer is acute lymphoblastic leukemia.

47. The method of embodiment 34 or 35, wherein the cancer is chronic myelogenous leukemia

48. The method of embodiment 34 or 35, wherein the cancer is lymphoma.

49. The method of embodiment 34 or 35, wherein the cancer is multiple myeloma.

50. The method of any one of embodiments 22-49, wherein the subject is a human.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I), or a pharmaceutically acceptable salt thereof: 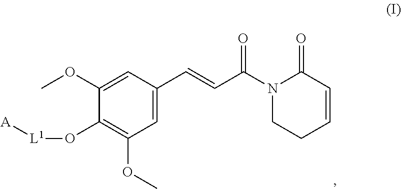
wherein:
A is a kinase inhibitor; and
L¹ is optionally substituted C₁-C₂₀ alkylene, optionally substituted C₁-C₂₀ heteroalkylene, optionally substituted C₁-C₂₀ alkenylene, optionally substituted C₁-C₂₀ heteroalkenylene, optionally substituted C₁-C₂₀ alkynylene, optionally substituted C₁-C₂₀ heteroalkynylene, optionally substituted C₃-C₁₄ carbocyclylene, or optionally substituted 3- to 14-membered heterocyclylene.

2. The compound of claim 1, or a pharmaceutically acceptable salt thereof, wherein A is a CDK inhibitor.

3. The compound of claim 2, or a pharmaceutically acceptable salt thereof, wherein A is a CDK9 or CDK10 inhibitor.

4. The compound of any one of claims 1-3, or a pharmaceutically acceptable salt thereof, wherein A is

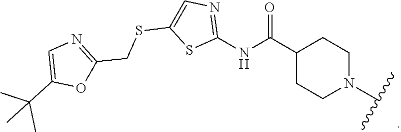 [SMILES: *N1CCC(C(=O)Nc2ncc(SCc3ncc(C(C)(C)C)o3)s2)CC1]

5. The compound of any one of claims 1-3, or a pharmaceutically acceptable salt thereof, wherein A is AT-7519, atuveciclib, AZD4573, BAY-1251152, CDKI-73, CDKI-73, dinaciclib, flavopiridol, i-CDK9, JSH-150, LDC000067, LY-2857785, NVP-2, RGB-286638, seliciclib, TG02, or zotiraciclib.

6. The compound of claim 1, or a pharmaceutically acceptable salt thereof, wherein A is an anaplastic lymphoma kinase inhibitor.

7. The compound of claim 6, or a pharmaceutically acceptable salt thereof, wherein A is

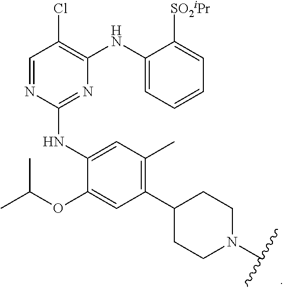

8. The compound of claim 6, or a pharmaceutically acceptable salt thereof, wherein A is alectinib, AP-26113, ASP-3026, brigatinib, CEP-37440, crizotinib, ensartinib, entrectinib, lorlatinib, NMS-E628, PF-06463922, TSR-011, X-376, or X-396.

9. The compound of any one of claims 1-8, or a pharmaceutically acceptable salt thereof, wherein L¹ is -(optionally substituted C₁-C₆ alkylene or optionally substituted C₁-C₆ heteroalkylene)₀₋₁-(optionally substituted 3- to 7-membered heterocyclylene)-(optionally substituted C₁-C₆ alkylene or optionally substituted C₁-C₆ heteroalkylene)₀₋₁-.

10. The compound of claim 9, or a pharmaceutically acceptable salt thereof, wherein L¹ is -(optionally substituted C₂ alkylene)-(optionally substituted monocyclic 6-membered para heterocyclylene)-(optionally substituted C₂ alkylene)-.

11. The compound of any one of claims 1-8, or a pharmaceutically acceptable salt thereof, wherein L¹ is optionally substituted C₁-C₁₅ alkylene, optionally substituted C₁-C₁₅ heteroalkylene, or optionally substituted 3- to 7-membered heterocyclylene.

12. The compound of any one of claims 1-11, or a pharmaceutically acceptable salt thereof, wherein L¹ is substituted with a carbonyl.

13. The compound of any one of claims 1-8, or a pharmaceutically acceptable salt thereof, wherein L¹ is

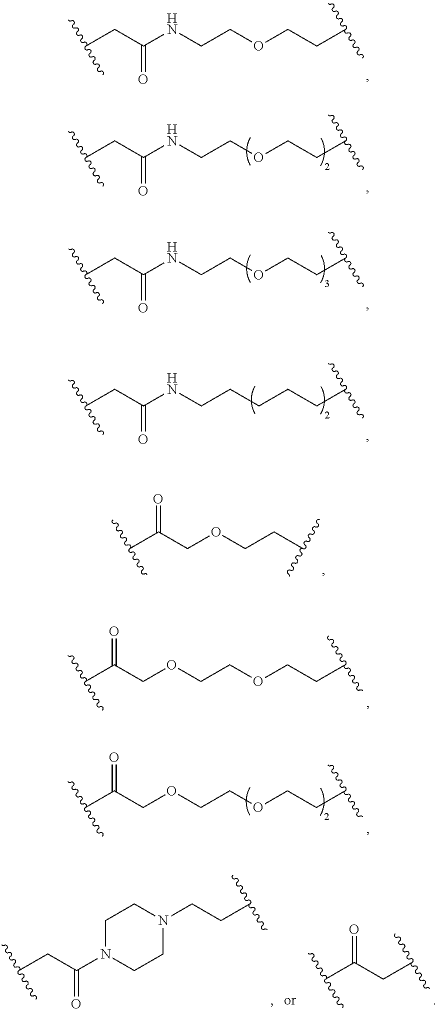

14. The compound of claim 1, wherein the compound is 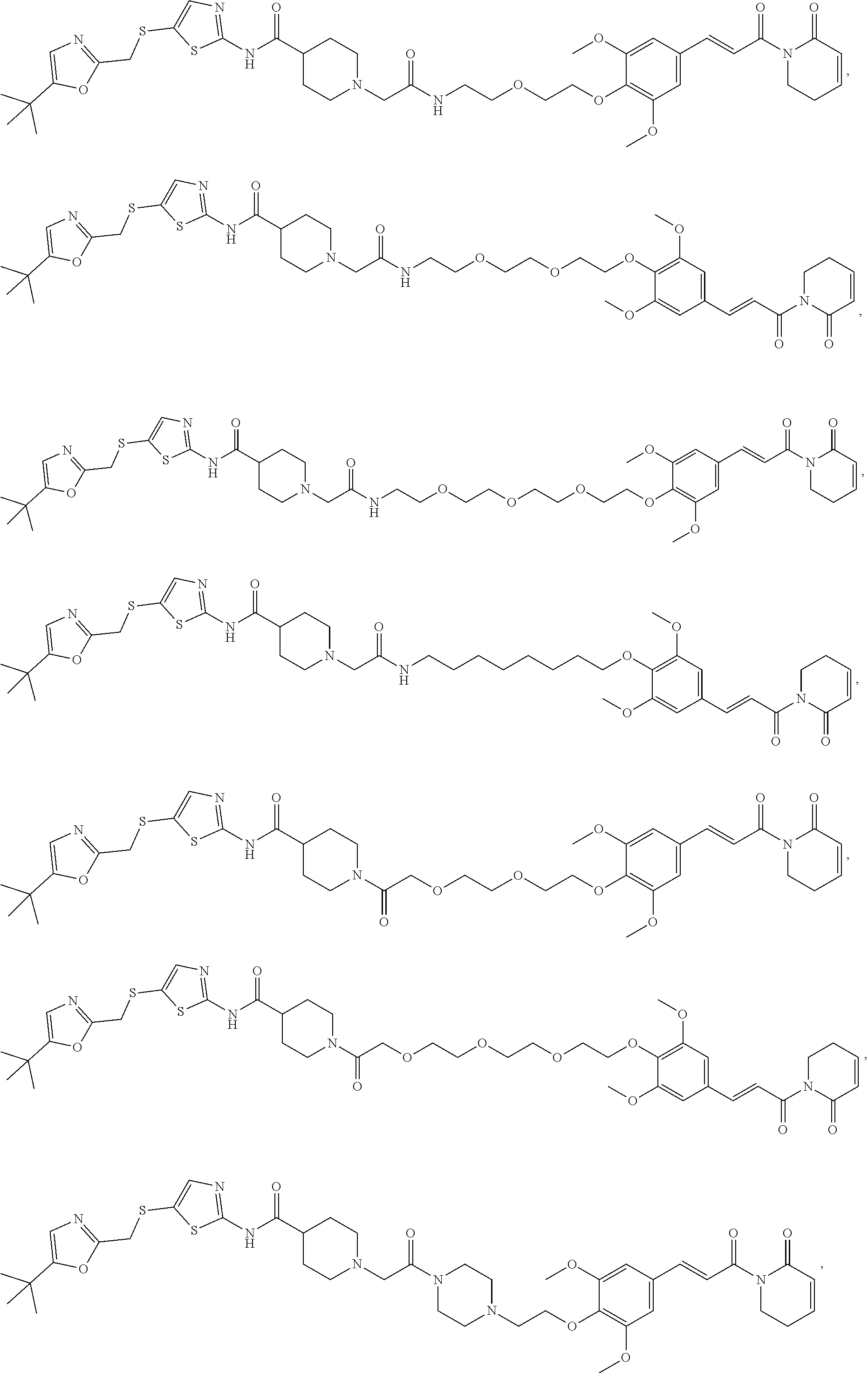
or a pharmaceutically acceptable salt thereof.

15. The compound of claim 1, wherein the compound is 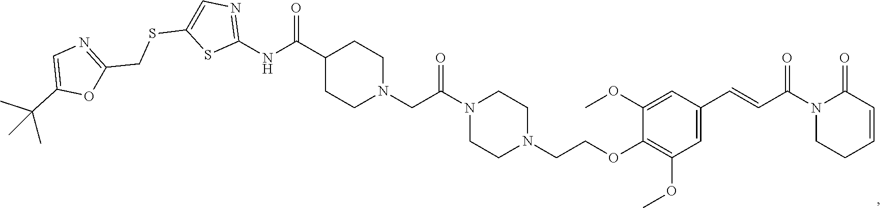 [SMILES: COc1cc(/C=C/C(=O)N2CCC=CC2=O)cc(OC)c1OCCN1CCN(C(=O)CN2CCC(C(=O)Nc3ncc(SCc4ncc(C(C)(C)C)o4)s3)CC2)CC1]
or a pharmaceutically acceptable salt thereof.

16. The compound of claim 1, wherein the compound is 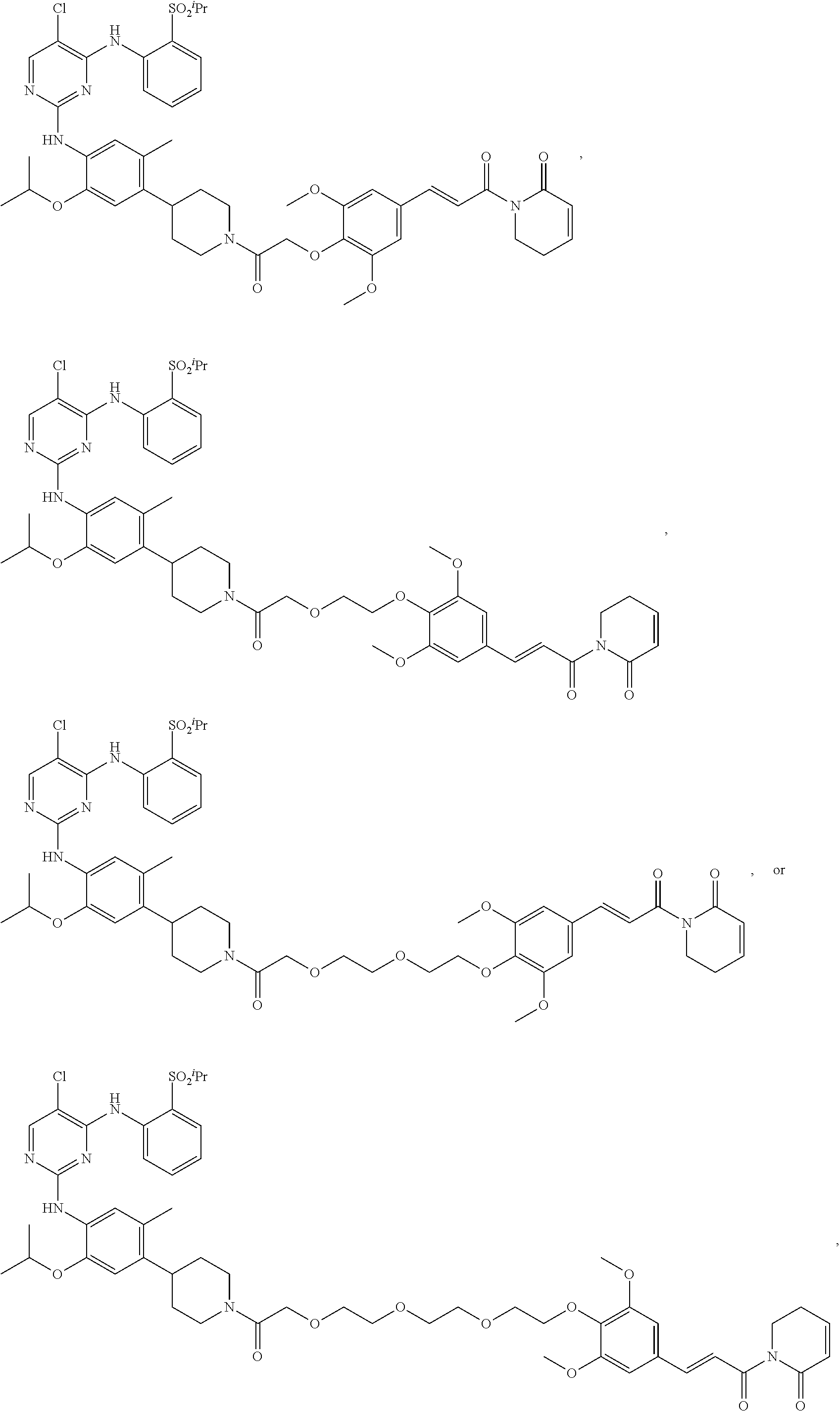
or a pharmaceutically acceptable salt thereof.

17. A pharmaceutical composition comprising a compound of any one of claims 1-16, or a pharmaceutically acceptable salt thereof, and a pharmaceutically acceptable excipient.

18. A method of inhibiting a kinase, the method comprising contacting a kinase with an effective amount of a compound of any one of claims 1-16, or a pharmaceutically acceptable salt thereof.

19. A method of degrading a kinase, the method comprising contacting a kinase with an effective amount of a compound of any one of claims 1-16, or a pharmaceutically acceptable salt thereof.

20. The method of claim 18 or 19, wherein the contacting is in vitro.

21. The method of claim 18 or 19, wherein the contacting in vivo.

22. The method of claim 18 or 19, further comprising administering the compound to a subject.

23. The method of any one of claims 18-22, wherein the kinase is CDK9 or CDK10.

24. The method of any one of claims 18-22, wherein the kinase is anaplastic lymphoma kinase.

25. The method of any one of claims 19-24, wherein the degrading is achieved in MOLT4 cells, 293T cells, K562 cells, LNCap cells, 22RV1 cells, PC3 cells, DU145 cells, or NCI-H2228 cells.

26. The method of any one of claims 19-25, wherein the degrading is achieved by recruitment of a cullin ring-related ubiquitin E3 ligase.

27. The method of claim 26, wherein the cullin ring-related ubiquitin E3 ligase is KEAP1.

28. A method of preventing or treating a disease or disorder in a subject in need thereof, the method comprising administering an effective amount of a compound of any one of claims 1-16, or a pharmaceutically acceptable salt thereof, or a pharmaceutical composition of claim 17.

29. A method of preventing or treating a subject suffering from or susceptible to a disease or disorder, the method comprising administering an effective amount of a compound of any one of claims 1-16, or a pharmaceutically acceptable salt thereof, or a pharmaceutical composition of claim 17.

30. The method of claim 28 or 29 of treating the disease or disorder or the subject.

31. The method of any one of claims 28-30, wherein the disease or disorder is associated with a CDK.

32. The method of claim 31, wherein the CDK is CDK9 or CDK10.

33. The method of any one of claims 28-32, wherein the disease or disorder is associated with anaplastic lymphoma kinase.

34. The method of any one of claims 28-33, wherein the disease is cancer.

35. The method of claim 34, wherein the cancer expresses KEAP1.

36. The method of claim 35, wherein the cancer is a leukemia that expresses KEAP1.

37. The method of claim 34 or 35, wherein the cancer is a solid tumor or liquid tumor.

38. The method of claim 34 or 35, wherein the cancer is lung cancer.

39. The method of claim 34 or 35, wherein the cancer is non-small cell lung cancer.

40. The method of claim 34 or 35, wherein the cancer is prostate cancer.

41. The method of claim 34 or 35, wherein the cancer is bone cancer, brain cancer, breast cancer, cervical cancer, colon cancer, esophageal cancer, head and neck cancer, kidney cancer, liver cancer, melanoma, NUT carcinoma, ovarian cancer, pancreatic cancer, or uterus cancer.

42. The method of claim 34 or 35, wherein the cancer is biliary tract cancer, bladder cancer, breast cancer, colorectal cancer, liver cancer, or stomach cancer.

43. The method of claim 34 or 35, wherein the cancer is breast cancer, colorectal cancer, esophageal cancer, glioblastoma, inflammatory myofibroblastic tumor, kidney cancer, neuroblastoma, ovarian cancer, pancreatic cancer, rhabdomyosarcoma, salivary gland cancer, or thyroid cancer.

44. The method of claim 34 or 35, wherein the cancer is a hematological malignancy.

45. The method of claim 34 or 35, wherein the cancer is leukemia.

46. The method of claim 34 or 35, wherein the cancer is acute lymphoblastic leukemia.

47. The method of claim 34 or 35, wherein the cancer is chronic myelogenous leukemia

48. The method of claim 34 or 35, wherein the cancer is lymphoma.

49. The method of claim 34 or 35, wherein the cancer is multiple myeloma.

50. The method of any one of claims 22-49, wherein the subject is a human.
